(19) United States
(12) Patent Application Publication
(10) Pub. No.: US 2026/0248831 A1
(43) Pub. Date: Aug. 27, 2026

(54) NON-TUBERCULOSIS MYCOBACTERIA INFECTION TREATMENT

(71) Applicant: AN2 Therapeutics, Inc., Menlo Park, CA (US)

(72) Inventors: Michael Richard Kevin ALLEY, Santa Clara, CA (US); Eric Edward EASOM, Atherton, CA (US); Paul Brian ECKBURG, Mountain View, CA (US); Kevin Michael KRAUSE, San Francisco, CA (US); George H. TALBOT, Bradenton, FL (US)

(73) Assignee: AN2 Therapeutics, Inc., Menlo Park, CA (US)

(21) Appl. No.: 18/716,804

(22) PCT Filed: May 18, 2022
(86) PCT No.: PCT/US2022/029887

§ 371 Date: Jun. 5, 2024

Related U.S. Application Data
(60) Provisional application No. 63/286,989, filed on Dec. 7, 2021.

(51) Int. Cl. A61K 31/69; A61K 9/00; A61K 31/133; A61K 31/395; A61K 31/7036; A61K 31/7048; A61K 31/7052; A61P 31/04

(52) U.S. Cl. CPC A61K 31/69; A61K 9/0053; A61K 31/133; A61K 31/395; A61K 31/7036; A61K 31/7048; A61K 31/7052; A61P 31/04

(57) ABSTRACT

The present invention is directed to methods of achieving a positive therapeutic response against an infection by non-tuberculosis Mycobacteria (NTM) through administering an oral dose of epetraborole to a human subject.

### CROSS REFERENCE TO RELATED APPLICATIONS

This application is a U.S. national stage application under 35 U.S.C. § 371 of International Application No. PCT/US2022/029887, filed internationally on May 18, 2022, which claims priority to and benefit of U.S. Provisional Patent Application No. 63/286,989 filed Dec. 7, 2021, each of which is hereby incorporated by reference, in its entirety.

### BACKGROUND OF THE INVENTION

There is a need for improved methods of treating non-tuberculosis Mycobacteria (NTM) infections in human subjects.

### BRIEF SUMMARY OF THE INVENTION

In one aspect, the invention provides a method of achieving a positive therapeutic response against an infection by non-tuberculosis Mycobacteria (NTM), comprising: administering epetraborole to a human subject in need of treatment thereof with a dosage regimen comprising: an oral dose of the epetraborole, of between about 250 mg and about 1000 mg, administered on the first day of the dosage regimen and the oral dose is administered daily or every other day, wherein the dosage regimen lasts until the positive therapeutic response is achieved.

### DETAILED DESCRIPTION OF THE INVENTION

### I. Definitions

In order that the application may be more completely understood, several definitions are set forth below. Such definitions are meant to encompass grammatical equivalents.

The term “about” in relation to a reference numerical value can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

The terms “a,” “an,” “the” and similar referents used in the context of describing the disclosure (especially in the context of the following claims) are to be construed to cover both the singular and the plural, unless otherwise indicated herein or clearly contradicted by context.

The following abbreviations have been used: ADH=Alcohol dehydrogenase; AE Adverse event; AESI=Adverse event of special interest; AIDS=Acquired immunodeficiency syndrome; ALIS=Amikacin liposome inhalation suspension; ALT=Alanine aminotransferase; AST=Aspartate aminotransferase; ATS=American Thoracic Society; AUC=Area under the concentration-time curve; AUC=Area under the concentration-time curve from time 0 to infinity; AUC₀₋₂₄=Area under the concentration-time curve from time 0 to 24 hours: CD4=Cluster of differentiation 4; CDAD=Clostridioides difficile-associated diarrhea; C. difficile=Clostridioides difficile; CL=Total body clearance from plasma; CLSI=Clinical and Laboratory Standards Institute; Cₘₐₓ=Maximum plasma drug concentration; Cₘᵢₙ=Minimum plasma drug concentration; CRA=Clinical Research Associate; CrCl=Creatinine clearance; CRO=Contract Research Organization; CT=Computed tomography; CTA=Clinical trial authorization; CTCAE Common Terminology Criteria for Adverse Events; CYP=Cytochrome P450; DSMB=Data and Safety Monitoring Board; ECG=Electrocardiogram; eCRF=Electronic case report form; EDC=Electronic data capture; EIU=Exposure In Utero; EOT=End of Therapy; ERS=European Respiratory Society; ESCMID=European Society of Clinical Microbiology and Infectious Diseases; FAS-PRO=Full Analysis Set-Patient-Reported Outcome; FDA=Food and Drug Administration; FOCP=Female patient of childbearing potential; GCP=Good Clinical Practice; GI=Gastrointestinal; HFM=Hollow-fiber macrophage; HIV=Human immunodeficiency virus; ICF=Informed consent form; ICH=International Council for Harmonisation; IDSA=Infectious Diseases Society of America; IEC=Independent Ethics Committee; IR=Immediate-release; IRB=Institutional Review Board; IRT=Interactive Response Technology; ITT=Intent-to-Treat; IV=Intravenous; LAR=Legally authorized representative; LFU=Late Follow-up; Log=Logarithmic; LPA=Line probe assay; MAC=Mycobacterium avium complex; M. avium=Mycobacterium avium; M. chimera=Mycobacterium chimaera; MCID=Minimal clinically important difference; MedDRA=Medical Dictionary for Regulatory Activities; MIC=Minimum inhibitory concentration; M. intracellulare=Mycobacterium intracellulare; NADPH=Nicotinamide adenine dinucleotide phosphate; NIMP=Non-investigational medical product; NTM=Nontuberculous mycobacteria(l); OBR=Optimized background regimen; PD=Pharmacodynamic(s); P-gp=P-glycoprotein; PK=Pharmacokinetic(s); PRO=Patient-Reported Outcome; PT=Preferred term; QD=Once daily; QTcF=QT interval corrected using Fridericia's formula; RBC=Red blood cell; rRNA=Ribosomal ribonucleic acid; SAE=Serious adverse event; SAP=Statistical Analysis Plan; SOC=System organ class; SUSAR=Suspected Unexpected Serious Adverse Reaction; tV=Elimination half-life; TEAE=Treatment-emergent adverse event; Tₘₐₓ=Time to reach maximum plasma drug concentration; tRNA=Transfer ribonucleic acid; ULN=pper limit of normal; Vd=Volume of distribution; Vₛₛ=Apparent volume of distribution at steady state.

“Epetraborole of the invention,” as used herein refers to epetraborole, salts (e.g. pharmaceutically acceptable salts), solvates and hydrates of these compounds.

“Moiety” refers to a radical of a molecule that is attached to the remainder of the molecule.

The symbol [symbol] , whether utilized as a bond or displayed perpendicular to a bond, indicates the point at which the displayed moiety is attached to the remainder of the molecule.

As used herein, the IC₅₀ refers to an amount, concentration or dosage of a particular test compound that achieves a 50% inhibition of a maximal response, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

As used herein, the term “positive therapeutic response” refers to the reduction or amelioration or elimination of the progression, severity and/or effect associated with an infection described herein, or the improvement in the infection condition, or the improvement in the disease associated with the infection, or the increase in the immune system response of the human subject, or the amelioration of one or more symptoms (preferably, one or more discernible symptoms) of an infection described herein resulting from the administration of one or more therapies. In specific embodiments, the term “positive therapeutic response” refers to the amelioration of at least one measurable physical parameter of an infection described herein, such as colony count, extent of infection, or severity of infection. In other embodiments the term “positive therapeutic response” refers to the inhibition of the progression of an infection described herein, either physically by, e.g., stabilization of a discernible symptom, physiologically by, e.g., stabilization of a physical parameter, or both. In other embodiments the term “positive therapeutic response” refers to an increase in the immune system response of the human subject. In an exemplary embodiment, achieving a positive therapeutic response against an infection described herein provides an improvement, or a lack of progression, in the disease associated with the infection, and/or an improvement, or a lack of progression, in the symptoms associated with the infection. For example, achieving a positive therapeutic response against an infection described herein refers to one or more of the following: (1) a reduction in the bacterial colony count; (2) a reduction in the extent of infection; (3) a reduction in the severity of infection; and (4) some relief from one or more other symptoms associated with the disease or condition. In some embodiments, achieving a positive therapeutic response against an infection described herein involves administering the compositions described herein for a pre-specified period of time, discontinuing administration for another specific period of time, and resuming administration of the compositions described herein for yet another specific period of time. In some embodiments, achieving a positive therapeutic response against an infection described herein involves administering the compositions described herein until one of the responses described herein is achieved, pausing administration of the compositions described herein while this positive therapeutic response continues to be observed, and resuming administration of the compositions described herein if this positive therapeutic response ceases to be observed. In an exemplary embodiment, achieving a positive therapeutic response is treating an infection described herein. In an exemplary embodiment, achieving a positive therapeutic response is treating a disease described herein.

A positive therapeutic response according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the treatment are outweighed by the therapeutically beneficial effects.

As used herein, the term ‘fasting condition’ can refer to when the human subject has had no food for at least 1 hour before study drug administration, or when the human subject has had no food for at least 2 hours after study drug administration, or when the human subject has had no food for at least 1 hour before and at least 2 hours after study drug administration.

As used herein, the term ‘in need of treatment’ can refer to when the human subject has an infection described herein, such as an NTM infection, by a clinical assessment and/or a microbiological assessment, and/or a radiological assessment. As used herein, the term ‘in need of treatment’ can refer to when the human subject has an infection described herein, such as an NTM infection, by a microbiological assessment and/or a radiological assessment. As used herein, the term ‘in need of treatment’ can refer to when the human subject has an infection described herein, such as an NTM infection, by a microbiological assessment. As used herein, the need of treatment can be assessed by analyzing a sputum sample, as described herein. As used herein, the need of treatment can be assessed by analyzing a deep bronchial sample, as described herein. As used herein, the need of treatment can be assessed by radiographic analysis of the human subject, as described herein. As used herein, the need of treatment can be assessed by a computed tomography (CT) scan of the chest of the human subject, as described herein. As used herein, the need of treatment can be assessed by a computed tomography (CT) scan of the lungs of the human subject, as described herein.

The amount of certain compounds described herein provided to the human subject can depend upon the weight of the human subject. The weight of the human subject can be obtained through conventional and commercially available means by a person, such as a medical professional (such as a doctor or nurse) or the human subject.

The terms “patient” and “human subject” can be used interchangeably herein.

### II. Overview

The invention provides methods of achieving a positive therapeutic response against an infection by non-tuberculosis Mycobacteria (NTM) through the oral administration of epetraborole according to a dosage regimen described herein.

### III. Epetraborole

Epetraborole has a structure according to the following formula:

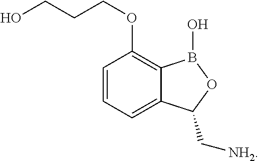 [SMILES: NC[C@H]1OB(O)c2c(OCCCO)cccc21]

Epetraborole can be produced according the methods such as those disclosed in PCT Pat Pub WO 2008/157726 (PCT Pat App PCT/US2008/07550); U.S. Pat. No. 7,816,344 (U.S. patent application Ser. No. 12/142,692); PCT Pat Pub WO 2011/127143 (PCT Pat App PCT/US2011/031384); and U.S. Pat. No. 9,243,003 (U.S. patent application Ser. No. 13/639,594), all of which are expressly incorporated herein by reference.

The epetraborole can form a hydrate with water, solvates with alcohols such as methanol, ethanol, propanol, and the like; adducts with amino compounds, such as ammonia, methylamine, ethylamine, and the like; adducts with acids, such as formic acid, acetic acid and the like; complexes with ethanolamine, quinoline, amino acids, and the like.

In an exemplary embodiment, the invention provides epetraborole, or a salt, hydrate or solvate thereof, or a combination thereof. In an exemplary embodiment, the invention provides epetraborole, or a salt, hydrate or solvate thereof. In an exemplary embodiment, the invention provides epetraborole, or a salt thereof. In an exemplary embodiment, the salt is a pharmaceutically acceptable salt. In an exemplary embodiment, the invention provides a hydrochloride salt of epetraborole. In an exemplary embodiment, the invention provides epetraborole, or a hydrate thereof. In an exemplary embodiment, the invention provides epetraborole, or a solvate thereof. In an exemplary embodiment, the epetraborole is epetraborole hydrochloride.

In an exemplary embodiment, epetraborole is administered in an amount of between about 200 mg and about 1,100 mg. In an exemplary embodiment, epetraborole is administered in an amount of between about 250 mg and about 1,000 mg. In an exemplary embodiment, epetraborole is administered in an amount of between about 200 mg and about 300 mg, or between about 225 mg and about 275 mg, or between about 240 mg or about 260 mg, or about 250 mg, or 250 mg. In an exemplary embodiment, epetraborole is administered in an amount of between about 400 mg and about 600 mg, or between about 450 mg and about 550 mg, or between about 475 tug and about 525 mg, or between about 490 mg or about 510 mg, or about 500 mg, or 500 mg. In an exemplary embodiment, epetraborole is administered in an amount of between about 600 mg and about 900 mg, or between about 650 mg and about 850 mg, or between about 700 mg and about 800 mg, or between about 725 mg or about 775 mg, or between about 740 mg or about 760 mg, or about 750 mg, or 750 mg. In an exemplary embodiment, epetraborole is administered in an amount of between about 900 mg and about 1,100 mg, or between about 925 mg and about 1,075 mg, or between about 950 tug and about 1,050 mg, or between about 975 tug or about 1,025 mg, or between about 990 mg or about 1,010 mg, or about 1,000 mg, or 1,000 mg. For any of the embodiments in this paragraph, the epetraborole is suitable for oral administration. For any of the embodiments in this paragraph, the epetraborole is a unit dose suitable for oral administration, for example, a single pill or capsule or tablet containing between about 475 mg and about 525 mg of epetraborole.

Once made, epetraborole can be administered to human subjects as described herein, such as in a dosage regimen described herein.

### III. a) Pharmaceutical Compositions and Pharmaceutical Administration

Epetraborole can be incorporated into pharmaceutical compositions suitable for administration to a human subject according to a dosage regimen described herein. Typically, the pharmaceutical composition comprises epetraborole and a pharmaceutically acceptable carrier. As used herein, “pharmaceutically acceptable carrier” includes any and all solvents, dispersion media, coatings, isotonic and absorption delaying agents, and the like that are physiologically compatible and are suitable for administration to a subject for the methods described herein.

In an exemplary embodiment, the pharmaceutical composition comprises epetraborole, and a filler. In an exemplary embodiment, the pharmaceutical composition comprises epetraborole and cellulose. In an exemplary embodiment, the pharmaceutical composition comprises epetraborole and microcrystalline cellulose.

In an exemplary embodiment, the pharmaceutical composition comprises epetraborole and a glidant. In an exemplary embodiment, the pharmaceutical composition comprises epetraborole and silicon dioxide. In an exemplary embodiment, the pharmaceutical composition comprises epetraborole and colloidal silicon dioxide.

In an exemplary embodiment, the pharmaceutical composition comprises epetraborole and a disintegrant. In an exemplary embodiment, the pharmaceutical composition comprises epetraborole and sodium. In an exemplary embodiment, the pharmaceutical composition comprises epetraborole and croscarmellose sodium.

In an exemplary embodiment, the pharmaceutical composition comprises epetraborole and a lubricant. In an exemplary embodiment, the pharmaceutical composition comprises epetraborole and a stearate. In an exemplary embodiment, the pharmaceutical composition comprises epetraborole and magnesium stearate.

In an exemplary embodiment, the pharmaceutical composition comprises epetraborole and a filler and a glidant. In an exemplary embodiment, the pharmaceutical composition comprises epetraborole and cellulose and silicon dioxide. In an exemplary embodiment, the pharmaceutical composition comprises epetraborole and microcrystalline cellulose and colloidal silicon dioxide.

In an exemplary embodiment, the pharmaceutical composition comprises epetraborole and a filler and a disintegrant. In an exemplary embodiment, the pharmaceutical composition comprises epetraborole and cellulose and sodium. In an exemplary embodiment, the pharmaceutical composition comprises epetraborole and microcrystalline cellulose and croscarmellose sodium.

In an exemplary embodiment, the pharmaceutical composition comprises epetraborole and a filler and a lubricant. In an exemplary embodiment, the pharmaceutical composition comprises epetraborole and cellulose and stearate. In an exemplary embodiment, the pharmaceutical composition comprises epetraborole and microcrystalline cellulose and magnesium stearate.

In an exemplary embodiment, the pharmaceutical composition comprises epetraborole and a glidant and a disintegrant. In an exemplary embodiment, the pharmaceutical composition comprises epetraborole and silicon dioxide and sodium. In an exemplary embodiment, the pharmaceutical composition comprises epetraborole and colloidal silicon dioxide and croscarmellose sodium.

In an exemplary embodiment, the pharmaceutical composition comprises epetraborole and a glidant and a lubricant. In an exemplary embodiment, the pharmaceutical composition comprises epetraborole and silicon dioxide and stearate. In an exemplary embodiment, the pharmaceutical composition comprises epetraborole and colloidal silicon dioxide and magnesium stearate.

In an exemplary embodiment, the pharmaceutical composition comprises epetraborole and a disintegrant and a lubricant. In an exemplary embodiment, the pharmaceutical composition comprises epetraborole and sodium and stearate. In an exemplary embodiment, the pharmaceutical composition comprises epetraborole and croscarmellose sodium and magnesium stearate.

In an exemplary embodiment, the pharmaceutical composition comprises epetraborole and a filler and a glidant and a disintegrant. In an exemplary embodiment, the pharmaceutical composition comprises epetraborole and cellulose and silicon dioxide and sodium. In an exemplary embodiment, the pharmaceutical composition comprises epetraborole and microcrystalline cellulose and colloidal silicon dioxide and croscarmellose sodium.

In an exemplary embodiment, the pharmaceutical composition comprises epetraborole and a filler and a glidant and a lubricant. In an exemplary embodiment, the pharmaceutical composition comprises epetraborole and cellulose and silicon dioxide and stearate. In an exemplary embodiment, the pharmaceutical composition comprises epetraborole and microcrystalline cellulose and colloidal silicon dioxide and magnesium stearate.

In an exemplary embodiment, the pharmaceutical composition comprises epetraborole and a glidant and a disintegrant and a lubricant. In an exemplary embodiment, the pharmaceutical composition comprises epetraborole and silicon dioxide and sodium and stearate. In an exemplary embodiment, the pharmaceutical composition comprises epetraborole and colloidal silicon dioxide and croscarmellose sodium and magnesium stearate.

In an exemplary embodiment, the pharmaceutical composition comprises epetraborole and a filler and a glidant and a disintegrant and a lubricant. In an exemplary embodiment, the pharmaceutical composition comprises epetraborole and cellulose and silicon dioxide and sodium and stearate. In an exemplary embodiment, the pharmaceutical composition comprises epetraborole and microcrystalline cellulose and colloidal silicon dioxide and croscarmellose sodium and magnesium stearate.

In an exemplary embodiment, the pharmaceutical composition comprises epetraborole in an amount of between about 200 mg and about 1,100 mg. In an exemplary embodiment, the pharmaceutical composition comprises epetraborole in an amount of between about 250 mg and about 1,000 mg. In an exemplary embodiment, the pharmaceutical composition comprises epetraborole in an amount of between about 200 mg and about 300 mg, or between about 225 mg and about 275 mg, or between about 240 mg or about 260 mg, or about 250 mg, or 250 mg. In an exemplary embodiment, the pharmaceutical composition comprises epetraborole in an amount of between about 400 mg and about 600 mg, or between about 450 mg and about 550 mg, or between about 475 mg and about 525 mg, or between about 490 mg or about 510 mg, or about 500 mg, or 500 mg. In an exemplary embodiment, the pharmaceutical composition comprises epetraborole in an amount of between about 600 mg and about 900 mg, or between about 650 mg and about 850 mg, or between about 700 mg and about 800 mg, or between about 725 mg or about 775 mg, or between about 740 mg or about 760 mg, or about 750 mg, or 750 mg. In an exemplary embodiment, the pharmaceutical composition comprises epetraborole in an amount of between about 900 mg and about 1,100 mg, or between about 925 mug and about 1,075 tug, or between about 950 mg and about 1,050 mg, or between about 975 mg or about 1,025 mg, or between about 990 mg or about 1,010 mg, or about 1,000 mg, or 1,000 mg. For any of the embodiments in this paragraph, the pharmaceutical composition is suitable for oral administration. For any of the embodiments in this paragraph, the pharmaceutical composition is a unit dose suitable for oral administration, for example, a single pill or capsule or tablet containing between about 475 mg and about 525 mg of epetraborole. For any of the embodiments in this paragraph, the pharmaceutical composition comprises epetraborole hydrochloride.

Epetraborole can be administered by a variety of methods known in the art. In an exemplary embodiment, the route/mode of administration is oral. As will be appreciated by the skilled artisan, the route and/or mode of administration will vary depending upon the desired results.

### IV. Other Non-Tuberculosis Mycobacteria Treatments

In an exemplary embodiment, epetraborole can be administered with other compounds to achieve a positive therapeutic response against a non-tuberculosis Mycobacterial infection. Examples of these other compounds are provided below:

### Ethambutol

Ethambutol has a structure according to the following formula:

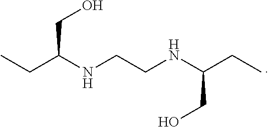

Ethambutol is commercially produced by a number of manufactuers, such as Barr Laboratories, Akorn, Sanofi, Cadila, Lupin, and Delmar. 100 mg and 400 mg tablets of ethambutol hydrochloride for oral administration are available.

PARAGRAPH Z: In an exemplary embodiment, the ethambutol or a hydrate, solvate, or pharmaceutically acceptable salt thereof, is administered in an oral dose of between about 10 mg/kg and about 30 mg/kg, or about 13 mg/kg or about 17 mg/kg, or about 15 mg/kg, or 15 mg/kg, or about 23 mg/kg or about 27 mg/kg, or about 25 mg/kg, or 25 mg/kg.

PARAGRAPH Y: In an exemplary embodiment, the ethambutol or a hydrate, solvate, or pharmaceutically acceptable salt thereof, is administered in an oral dose of between of between about 13 mg/kg and about 17 mg/kg, or about 15 mg/kg, administered daily. In an exemplary embodiment, the ethambutol or a hydrate, solvate, or pharmaceutically acceptable salt thereof, of between about 600 mg and about 2000 mg, or about 600 mg and about 800 mg, or about 800 mg and about 1,000 mg, or about 1,000 mg and about 1,200 mg, or about 1,200 mg and about 1,400 mg, or about 1,400 mg and about 1,600 mg, or about 1,600 mg and about 1,800 mg, or about 1,800 mg and about 2,000 mg, administered daily.

PARAGRAPH X: In an exemplary embodiment, the ethambutol or a hydrate, solvate, or pharmaceutically acceptable salt thereof, is administered in an oral dose of between of between about 23 mg/kg and about 27 mg/kg, or about 25 mg/kg, administered three days a week. In an exemplary embodiment, the ethambutol or a hydrate, solvate, or pharmaceutically acceptable salt thereof, is administered in an oral dose of between about 900 tug and about 3,400 mg, or about 900 mg and about 1,200 mg, or about 1,200 mg and about 1,500 mg, or about 1,500 mg and about 1,800 mg, or about 1,800 mg and about 2,100 mg, or about 2,100 mg and about 2,400 mg, 2,400 mg and about 2,700 mg, or about 2,700 mg and about 3,000 mg, or about 3,000 mg and about 3,400 mg, administered three days a week.

### Clarithromycin or Azithromycin

Azithromycin is commercially produced by a number of manufactuers, such as Pfizer, Sandoz, Teva, Alembic, Lupin, Fresenius, Hospira, and Aurobindo. 250 mg, 500 mg, and 600 mg tablets of azithromycin for oral administration are available. 500 mg/vial of azithromycin for intravenous administration are available. 100 mg/5 mL suspension of azithromycin for oral administration for available.

PARAGRAPH W: In an exemplary embodiment, the azithromycin is administered in an oral dose of between about 200 mg and about 550 mg, or about 200 mg and about 300 mg, or about 225 mg and about 275 mg, or about 250 mg, or about 250 mg, or between about 450 mg and about 550 mg, or about 475 mg and about 525 mg, or about 500 mg, or 500 mg. In an exemplary embodiment, the oral dose of azithromycin or a hydrate, solvate, or pharmaceutically acceptable salt thereof, of between about 200 mg and about 550 mg, administered daily or three days a week.

PARAGRAPH V: In an exemplary embodiment, the oral dose of azithromycin or a hydrate, solvate, or pharmaceutically acceptable salt thereof, of between about 225 mg and about 275 tug, or about 250 tug, administered daily. In an exemplary embodiment, the oral dose of azithromycin or a hydrate, solvate, or pharmaceutically acceptable salt thereof, of between about 475 mg and about 525 mg, or about 500 mg, administered daily.

PARAGRAPH U: In an exemplary embodiment, the oral dose of azithromycin or a hydrate, solvate, or pharmaceutically acceptable salt thereof, of between about 475 mg and about 525 tug, or about 500 tug, administered three days a week.

Clarithromycin is commercially produced by a number of manufactuers, such as Sandoz, Century Pharmaceuticals, Teva, Wockhardt, and Alembic. 250 mg and 500 mg tablets of clarithromycin for oral administration are available. 125 mg/5 mL or 250 mg/5 mL suspension of clarithromycin for oral administration for available.

PARAGRAPH T: In an exemplary embodiment, the clarithromycin is administered in an oral dose of between about of between about 400 mg and about 600 mg, or about 450 mg and about 550 mg, or about 475 mg and about 525 mg, or about 500 mug, or 500 mg or between about 900 mg and about 1,100 mg, or about 975 mg or about 1,025 mg, or about 1,000 mg, or 1,000 mg. In an exemplary embodiment, the clarithromycin is administered in an oral dose of between about 950 mg and about 1,050 mg daily, or about 1,000 daily, or about 400 tug and about 600 mg twice per day, or about 450 tug and about 550 mg twice per day, or about 475 mg and about 525 mg twice per day, or about 500 mg twice per day, or 500 tug twice per day. In an exemplary embodiment, the oral dose of clarithromycin or a hydrate, solvate, or pharmaceutically acceptable salt thereof, of between about 900 mg and about 1,100 mg, or about 975 mg or about 1,025 mg, or about 1,000 mg, or 1,000 mg, administered daily or three days a week. In an exemplary embodiment, the oral dose of clarithromycin or a hydrate, solvate, or pharmaceutically acceptable salt thereof, of between about 400 mg and about 600 mg twice per day, or about 450 tug and about 550 mg twice per day, or about 475 mg and about 525 mg twice per day, or about 500 mg twice per day, or 500 mg twice per day, administered daily or three days a week.

PARAGRAPH S: In an exemplary embodiment, the oral dose of clarithromycin or a hydrate, solvate, or pharmaceutically acceptable salt thereof, of between about 900 mg and about 1,100 tug, or about 975 mg or about 1,025 mg, or about 1,000 mg, or 1,000 tug, administered daily. In an exemplary embodiment, the oral dose of clarithromycin or a hydrate, solvate, or pharmaceutically acceptable salt thereof, of between about 400 mg and about 600 mg twice per day, or about 450 mg and about 550 mg twice per day, or about 475 mg and about 525 mg twice per day, or about 500 mg twice per day, or 500 mg twice per day, administered daily.

PARAGRAPH R: In an exemplary embodiment, the oral dose of clarithromycin or a hydrate, solvate, or pharmaceutically acceptable salt thereof, of between about 900 mg and about 1,100 mg, or about 975 mg or about 1,025 mg, or about 1,000 tug, or 1,000 mg, administered three days a week. In an exemplary embodiment, the oral dose of clarithromycin or a hydrate, solvate, or pharmaceutically acceptable salt thereof, of between about 400 mg and about 600 mg twice per day, or about 450 mg and about 550 mg twice per day, or about 475 mg and about 525 tug twice per day, or about 500 tug twice per day, or 500 mg twice per day, administered three days a week.

### Rifanpicin

Rifampicin, also known as rifampin, is commercially produced by a number of manufactuers, such as Akorn, Lupin, Epic Pharma, Oxford, Fresenius, Sanofi Aventis, Novartis, Otto Brandes, Arudavis Labs, and Sichuan Long March Pharma. 150 tug and 300 mg capsules of rifampicin for oral administration are available. 600 mg/vial of rifampicin for intravenous administration are available.

PARAGRAPH Q: In an exemplary embodiment, the rifampicin is administered in an oral dose of between about 5 mg/kg and about 15 mg/kg, or between about 9 mg/kg or about 11 mg/kg, or about 10 mg/kg, or 10 mg/kg. In an exemplary embodiment, the rifampicin is administered in an oral dose of between about 350 mg and about 700 mg, or about 350 and about 500 mg, or about 425 mg and about 475 mg, or about 440 mg and about 460 mg, or about 450 mg or 450 mg, or about 500 mg and about 700 mg, or about 550 mg and about 650 mg, or about 575 mg and about 625 mg, or about 590 mg and about 610 mg, or about 600 mg, or 600 mg. In an exemplary embodiment, the oral dose of rifampicin or a hydrate, solvate, or pharmaceutically acceptable salt thereof, of between about 9 mg/kg or about 11 mg/kg, or about 10 mg/kg, administered daily or three days a week.

PARAGRAPH P: In an exemplary embodiment, the oral dose of rifampicin or a hydrate, solvate, or pharmaceutically acceptable salt thereof, of between about 9 mg/kg or about 11 mg/kg, or about 10 mg/kg, administered daily. In an exemplary embodiment, the oral dose of rifampicin or a hydrate, solvate, or pharmaceutically acceptable salt thereof, of between about 425 tug and about 475 mg, or about 440 mg and about 460 tug, or about 450 mg, administered daily. In an exemplary embodiment, the oral dose of rifampicin or a hydrate, solvate, or pharmaceutically acceptable salt thereof, of between about 575 mg and about 625 mg, or about 590 mg and about 610 mg, or about 600 mg, administered daily.

PARAGRAPH O: In an exemplary embodiment, the oral dose of rifampicin or a hydrate, solvate, or pharmaceutically acceptable salt thereof, of between about 575 mg and about 625 mg, or about 590 mg and about 610 mg, or about 600 mg, administered three days a week.

### Rifabutin

Rifabutin is commercially produced by a number of manufactuers, such as Pfizer, Chongqing Huapont Pharma, Lupin, Novitium Pharmaand Guangzhou Tosun Pharma. 150 mg capsules of rifabutin for oral administration are available.

PARAGRAPH N: In an exemplary embodiment, the rifabutin is administered in an oral dose of between about 100 mg and about 350 mg, or about 100 and about 200 mg, or about 125 mg and about 175 mg, or about 140 mg and about 160 mg, or about 150 mg or 150 mg, or about 250 mg and about 350 mg, or about 275 mg and about 325 mg, or about 290 mg and about 310 mg, or about 300 mg, or 300 mg.

PARAGRAPH M: In an exemplary embodiment, the oral dose of rifabutin or a hydrate, solvate, or pharmaceutically acceptable salt thereof, of between about 425 mg and about 475 mg, or about 440 mg and about 460 mg, or about 450 mg, administered daily. In an exemplary embodiment, the oral dose of rifabutin or a hydrate, solvate, or pharmaceutically acceptable salt thereof, of between about 575 mg and about 625 mg, or about 590 mg and about 610 tug, or about 600 mg, administered daily.

PARAGRAPH L: In an exemplary embodiment, the oral dose of rifabutin or a hydrate, solvate, or pharmaceutically acceptable salt thereof, of between about 575 mg and about 625 mg, or about 590 tug and about 610 tug, or about 600 mg, administered three days a week.

### Amikacin, Injected

Amikacin is commercially available in injectable formulations from a number of manufactuers, such as Teva, Hospira, Abbott Laboratories, Apothecon, and Fresenius. 50 mg/mL and 250 mg/mL of amikacin sulfate for intravenous administration are available.

PARAGRAPH K: In an exemplary embodiment, the intravenous dose of amikacin, is between about 8 mg/kg or about 17 mg/kg, or about 10 mg/kg or about 15 mg/kg, or about 13 mg/kg or about 17 mg/kg, or about 10 mg/kg, or 10 mg/kg, or about 15 mg/kg, or 15 mg/kg, administered daily.

PARAGRAPH J: In an exemplary embodiment, the intravenous dose of amikacin, of between about 13 mg/kg or about 27 mg/kg, or about 15 mg/kg or about 25 mg/kg, or about 15 mg/kg, or 15 mg/kg, or about 25 mg/kg, or 25 mg/kg, administered three days a week.

### Amikacin, Inhaled

An inhaled liposomal suspension of amikacin (ARIKAYCE®) is commercially available from Insmed, Inc. 590 mg/8.4 mL formulations for inhalation are available.

PARAGRAPH I: In an exemplary embodiment, the inhaled dose of amikacin is between about 580 mg and about 600 mg, or about 590 mg, or 590 mg, administered daily.

### Ciprofloxacin

Ciprofloxacin is commercially produced by a number of manufactuers, such as Bayer, Dr. Reddy's, Aurobindo, Taro, and Watson. 100 mg, 250 mg and 500 mg tablets of ciprofloxacin hydrochloride for oral administration are available.

### Linezolid

Linezolid is commercially produced by a number of manufactuers, such as Alembic, Pfizer, Glenmark and Zydus. 600 mg tablets of linezolid for oral administration are available.

### Moxifloxacin

Moxifloxacin is commercially produced by a number of manufactuers, such as Teva, Dr. Reddy's, and Aurobindo. 400 mg tablets of moxifloxacin hydrochloride for oral administration are available.

### Streptomycin

Streptomycin is commercially produced by Xgen Pharmaceuticals. 1 g/vial of streptomycin sulfate for intravenous administration is available.

### Tigecycline

Tigecycline is commercially produced by a number of manufactuers, such as Prism, Sandoz, Fresenius, and Apotex. 50 mg/vial of tigecycline for intravenous administration is available.

Synergistic Effects with Epetraborole and Ethambutol

Synergistic effects between epetraborole and ethambutol have been discovered which can result in more effective treatments of NTM infections.

### V. Administering/Dosage regimen with Epetraborole

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, thereby achieving the positive therapeutic response. For any of the embodiments in Section V, the epetraborole is epetraborole hydrochloride. In an exemplary embodiment, the epetraborole or a hydrate, solvate, or pharmaceutically acceptable salt thereof, is administered in an amount as described herein. In an exemplary embodiment, for any method described herein, the positive therapeutic response is treating the non-tuberculosis Mycobacteria (NTM) infection. In an exemplary embodiment, for any dosage regimen described herein, the positive therapeutic response is treating the non-tuberculosis Mycobacteria (NTM) infection. In an exemplary embodiment, for any dosage regimen described herein, the epetraborole is epetraborole hydrochloride. For any of the embodiments in Section V, the epetraborole is epetraborole hydrochloride.

In an exemplary embodiment, a composition described herein can be used to achieve a positive therapeutic response against infection by non-tuberculosis Mycobacteria (NTM). In an exemplary embodiment, a combination of compositions described herein can be used to achieve a positive therapeutic response against infection by non-tuberculosis Mycobacteria (NTM). In an exemplary embodiment, an epetraborole composition is administered according to a method described herein to achieve a positive therapeutic response against infection by non-tuberculosis Mycobacteria (NTM). In an exemplary embodiment, the infection by non-tuberculosis, Mycobacteria (NTM) is receptive to treatment by epetraborole.

### V. i.) Epetraborole & Ethambutol

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, and ethambutol or a hydrate, solvate, or pharmaceutically acceptable salt thereof, thereby achieving the positive therapeutic response. In an exemplary embodiment, the epetraborole or a hydrate, solvate, or pharmaceutically acceptable salt thereof, is administered in an oral dose of between about 250 mg and about 1000 mg or about 450 mg and about 550 tug, or about 490 tug and about 510 mg, or about 500 mg, or 500 mg, and the ethambutol is administered in an oral dose of between about 10 mg/kg and about 30 mg/kg, or between about 13 mg/kg or about 17 mg/kg, or about 15 Mg/kg, or 15 mg/kg, or between about 23 mg/kg or about 27 mg/kg, or about 25 mg/kg, or 25 mg/kg.

In an exemplary embodiment, the invention provides a method of achieving a positive therapeutic response against a non-tuberculosis Mycobacteria (NTM) infection comprising administering to a human subject in need of treatment thereof, a dosage regimen comprising: an oral dose amount as described herein of the epetraborole or a hydrate, solvate, or pharmaceutically acceptable salt thereof, administered daily or every other day; and an oral dose amount as described herein of the ethambutol or a hydrate, solvate, or pharmaceutically acceptable salt thereof, administered daily or three days a week.

In an exemplary embodiment, the dosage regimen comprises an oral dose of epetraborole or a hydrate, solvate, or pharmaceutically acceptable salt thereof, of between about 250 mg and about 1000 mg, administered daily, and an oral dose of ethambutol or a hydrate, solvate, or pharmaceutically acceptable salt thereof, of between about 10 mg/kg and about 30 mg/kg, administered daily or three days a week. In an exemplary embodiment, the dosage regimen comprises an oral dose of epetraborole or a hydrate, solvate, or pharmaceutically acceptable salt thereof, of between about 475 mg and about 525 mg, or about 500 mg, administered daily, and an oral dose of ethambutol or a hydrate, solvate, or pharmaceutically acceptable salt thereof, of between about 13 mg/kg and about 17 mg/kg, or about 15 mg/kg, administered daily. In an exemplary embodiment, the dosage regimen comprises an oral dose of epetraborole or a hydrate, solvate, or pharmaceutically acceptable salt thereof, of between about 475 mg and about 525 mg, or about 500 mg, administered daily, and an oral dose of ethambutol or a hydrate, solvate, or pharmaceutically acceptable salt thereof, of between about 600 mg and about 2000 mg, or about 600 mg and about 800 mg, or about 800 mg and about 1,000 mg, or about 1,000 mg and about 1,200 mg, or about 1,200 mg and about 1,400 mg, or about 1,400 mg and about 1,600 mg, or about 1,600 mg and about 1,800 mg, or about 1,800 mg and about 2,000 mg, administered daily. In an exemplary embodiment, the dosage regimen comprises an oral dose of epetraborole or a hydrate, solvate, or pharmaceutically acceptable salt thereof, of between about 475 mg and about 525 mg, or about 500 mg, administered daily, and an oral dose of ethambutol or a hydrate, solvate, or pharmaceutically acceptable salt thereof, of between about 23 mg/kg and about 27 mg/kg, or about 25 mg/kg, administered three days a week. In an exemplary embodiment, the dosage regimen comprises an oral dose of epetraborole or a hydrate, solvate, or pharmaceutically acceptable salt thereof, of between about 475 mg and about 525 mg, or about 500 mg, administered daily, and an oral dose of ethambutol or a hydrate, solvate, or pharmaceutically acceptable salt thereof, of between about 900 mg and about 3,400 mg, or about 900 mg and about 1,200 mg, or about 1,200 mg and about 1,500 mg, or about 1,500 mg and about 1,800 mg, or about 1,800 mg and about 2,100 mg, or about 2,100 mg and about 2,400 mg, 2,400 mg and about 2,700 mg, or about 2,700 mg and about 3,000 mg, or about 3,000 mg and about 3,400 mg, administered three days a week.

### V. ii.) Epetraborole & Azithromycin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, and azithromycin or a hydrate, solvate, or pharmaceutically acceptable salt thereof, thereby achieving the positive therapeutic response. In an exemplary embodiment, the epetraborole or a hydrate, solvate, or pharmaceutically acceptable salt thereof, is administered in an oral dose of between about 250 mg and about 1000 mg or about 450 mg and about 550 mg, or about 490 mg and about 510 mg, or about 500 mg, or 500 mg, and the azithromycin is administered in an oral dose of between about 200 mg and about 550 mg, or about 200 mg and about 300 mg, or about 225 mg and about 275 mg, or about 250 mg, or about 250 mg, or between about 450 mg and about 550 mg, or about 475 mg and about 525 mg, or about 500 mg, or 500 mg.

In an exemplary embodiment, the invention provides a method of achieving a positive therapeutic response against a non-tuberculosis Mycobacteria (NTM) infection comprising administering to a human subject in need of treatment thereof, a dosage regimen comprising: an oral dose amount as described herein of the epetraborole or a hydrate, solvate, or pharmaceutically acceptable salt thereof, administered daily or every other day; and an oral dose amount as described herein of the azithromycin or a hydrate, solvate, or pharmaceutically acceptable salt thereof, administered daily or three days a week.

In an exemplary embodiment, the dosage regimen comprises an oral dose of epetraborole or a hydrate, solvate, or pharmaceutically acceptable salt thereof, of between about 250 mg and about 1000 mg, administered daily, and an oral dose of azithromycin or a hydrate, solvate, or pharmaceutically acceptable salt thereof, of between about 200 mg and about 550 mg, administered daily or three days a week. In an exemplary embodiment, the dosage regimen comprises an oral dose of epetraborole or a hydrate, solvate, or pharmaceutically acceptable salt thereof, of between about 475 mg and about 525 mg, or about 500 mg, administered daily, and an oral dose of azithromycin or a hydrate, solvate, or pharmaceutically acceptable salt thereof, of between about 225 mg and about 275 mg, or about 250 mg, administered daily. In an exemplary embodiment, the dosage regimen comprises an oral dose of epetraborole or a hydrate, solvate, or pharmaceutically acceptable salt thereof, of between about 475 mg and about 525 mg, or about 500 mg, administered daily, and an oral dose of azithromycin or a hydrate, solvate, or pharmaceutically acceptable salt thereof, of between about 475 mg and about 525 mg, or about 500 mg, administered daily. In an exemplary embodiment, the dosage regimen comprises an oral dose of epetraborole or a hydrate, solvate, or pharmaceutically acceptable salt thereof, of between about 475 mg and about 525 mg, or about 500 mg, administered daily, and an oral dose of azithromycin or a hydrate, solvate, or pharmaceutically acceptable salt thereof, of between about 475 mg and about 525 mg, or about 500 mg, administered three days a week.

### V. iii.) Epetraborole & Clarithromycin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, and clarithromycin or a hydrate, solvate, or pharmaceutically acceptable salt thereof, thereby achieving the positive therapeutic response. In an exemplary embodiment, the epetraborole or a hydrate, solvate, or pharmaceutically acceptable salt thereof, is administered in an oral dose of between about 250 mg and about 1000 mg or about 450 mg and about 550 mg, or about 490 mg and about 510 tug, or about 500 mg, or 500 tug, and the clarithromycin is administered in an oral dose of between about 400 mg and about 600 mg, or about 450 mg and about 550 mg, or about 475 mg and about 525 mg, or about 500 mg, or 500 mg or between about 900 mg and about 1,100 mg, or about 975 mg or about 1,025 mg, or about 1,000 mg, or 1,000 mg. In an exemplary embodiment, the epetraborole or a hydrate, solvate, or pharmaceutically acceptable salt thereof, is administered in an oral dose of between about 250 mg and about 1000 mg or between about 450 mg and about 550 mg, or between about 490 mg and about 510 mg, or about 500 mg, or 500 mg, and the clarithromycin is administered in an oral dose of between about 950 mg and about 1,050 mg daily, or about 1,000 daily, or about 400 mg and about 600 mg twice per day, or about 450 mg and about 550 mg twice per day, or about 475 tug and about 525 mg twice per day, or about 500 tug twice per day, or 500 tug twice per day.

In an exemplary embodiment, the invention provides a method of achieving a positive therapeutic response against a non-tuberculosis Mycobacteria (NTM) infection comprising administering to a human subject in need of treatment thereof, a dosage regimen comprising: an oral dose amount as described herein of the epetraborole or a hydrate, solvate, or pharmaceutically acceptable salt thereof, administered daily or every other day; and an oral dose amount as described herein of the clarithromycin or a hydrate, solvate, or pharmaceutically acceptable salt thereof, administered daily or three days a week.

In an exemplary embodiment, the dosage regimen comprises an oral dose of epetraborole or a hydrate, solvate, or pharmaceutically acceptable salt thereof, of between about 250 mg and about 1000 mg, administered daily, and an oral dose of clarithromycin or a hydrate, solvate, or pharmaceutically acceptable salt thereof, of between about 900 mg and about 1,100 mg, or about 975 mg or about 1,025 mg, or about 1,000 mg, or 1,000 mg, administered daily or three days a week.

In an exemplary embodiment, the dosage regimen comprises an oral dose of epetraborole or a hydrate, solvate, or pharmaceutically acceptable salt thereof, of between about 250 mg and about 1000 mg, administered daily, and an oral dose of clarithromycin or a hydrate, solvate, or pharmaceutically acceptable salt thereof, of between about 400 mg and about 600 mg twice per day, or about 450 mg and about 550 mg twice per day, or about 475 mg and about 525 mg twice per day, or about 500 mg twice per day, or 500 mg twice per day, administered daily or three days a week. In an exemplary embodiment, the dosage regimen comprises an oral dose of epetraborole or a hydrate, solvate, or pharmaceutically acceptable salt thereof, of between about 475 mg and about 525 mg, or about 500 mg, administered daily, and an oral dose of clarithromycin or a hydrate, solvate, or pharmaceutically acceptable salt thereof, of between about 900 mg and about 1,100 mg, or about 975 mg or about 1,025 mg, or about 1,000 mg, or 1,000 mg, administered daily. In an exemplary embodiment, the dosage regimen comprises an oral dose of epetraborole or a hydrate, solvate, or pharmaceutically acceptable salt thereof, of between about 475 mg and about 525 mg, or about 500 mg, administered daily, and an oral dose of clarithromycin or a hydrate, solvate, or pharmaceutically acceptable salt thereof, of between about 900 mg and about 1,100 mg, or about 975 mg or about 1,025 mg, or about 1,000 mg, or 1,000 mg, administered three days a week. In an exemplary embodiment, the dosage regimen comprises an oral dose of epetraborole or a hydrate, solvate, or pharmaceutically acceptable salt thereof, of between about 475 mg and about 525 mug, or about 500 mg, administered daily, and an oral dose of clarithromycin or a hydrate, solvate, or pharmaceutically acceptable salt thereof, of between about 400 mg and about 600 mg twice per day, or about 450 mg and about 550 mg twice per day, or about 475 mg and about 525 mg twice per day, or about 500 mg twice per day, or 500 mg twice per day, administered daily. In an exemplary embodiment, the dosage regimen comprises an oral dose of epetraborole or a hydrate, solvate, or pharmaceutically acceptable salt thereof of between about 475 mg and about 525 mg, or about 500 mg, administered daily, and an oral dose of clarithromycin or a hydrate, solvate, or pharmaceutically acceptable salt thereof, of between about 400 mg and about 600 mg twice per day, or about 450 mg and about 550 mg twice per day, or about 475 mg and about 525 mg twice per day, or about 500 mg twice per day, or 500 mg twice per day, administered three days a week.

### V. iv.) Epetraborole & Rifampicin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, and rifampicin or a hydrate, solvate, or pharmaceutically acceptable salt thereof, thereby achieving the positive therapeutic response. In an exemplary embodiment, the epetraborole or a hydrate, solvate, or pharmaceutically acceptable salt thereof, is administered in an oral dose of between about 250 mg and about 1000 mg or about 450 mg and about 550 tug, or about 490 tug and about 510 mg, or about 500 mg, or 500 mg, and the rifampicin is administered in an oral dose of between about 5 Mg/kg and about 15 mg/kg, or between about 9 mg/kg or about 11 mg/kg, or about 10 mg/kg, or 10 mg/kg. In an exemplary embodiment, the epetraborole or a hydrate, solvate, or pharmaceutically acceptable salt thereof, is administered in an oral dose of between about 250 mg and about 1000 mg or between about 450 mg and about 550 mg, or between about 490 mg and about 510 mg, or about 500 mg, or 500 mg, and the rifampicin is administered in an oral dose of between about 350 mg and about 700 mg, or about 350 and about 500 mg, or about 425 mg and about 475 mg, or about 440 mg and about 460 mg, or about 450 mg or 450 mg, or about 500 mg and about 700 mg, or about 550 mg and about 650 mg, or about 575 mg and about 625 mg, or about 590 mg and about 610 mg, or about 600 mg, or 600 mg.

In an exemplary embodiment, the invention provides a method of achieving a positive therapeutic response against a non-tuberculosis Mycobacteria (NTM) infection comprising administering to a human subject in need of treatment thereof, a dosage regimen comprising: an oral dose amount as described herein of the epetraborole or a hydrate, solvate, or pharmaceutically acceptable salt thereof, administered daily or every other day; and an oral dose amount as described herein of the rifampicin or a hydrate, solvate, or pharmaceutically acceptable salt thereof, administered daily or three days a week.

In an exemplary embodiment, the dosage regimen comprises an oral dose of epetraborole or a hydrate, solvate, or pharmaceutically acceptable salt thereof, of between about 250 mg and about 1000 mg, administered daily, and an oral dose of rifampicin or a hydrate, solvate, or pharmaceutically acceptable salt thereof, of between about 9 mg/kg or about 11 mg/kg, or about 10 mg/kg, administered daily or three days a week. In an exemplary embodiment, the dosage regimen comprises an oral dose of epetraborole or a hydrate, solvate, or pharmaceutically acceptable salt thereof, of between about 475 mg and about 525 mg, or about 500 mg, administered daily, and an oral dose of rifampicin or a hydrate, solvate, or pharmaceutically acceptable salt thereof, of between about 9 mg/kg or about 11 mg/kg, or about 10 mg/kg, administered daily.

In an exemplary embodiment, the dosage regimen comprises an oral dose of epetraborole or a hydrate, solvate, or pharmaceutically acceptable salt thereof, of between about 475 mg and about 525 mg, or about 500 mg, administered daily, and an oral dose of rifampicin or a hydrate, solvate, or pharmaceutically acceptable salt thereof, of between about 425 mg and about 475 mg, or about 440 mg and about 460 mg, or about 450 mg, administered daily.

In an exemplary embodiment, the dosage regimen comprises an oral dose of epetraborole or a hydrate, solvate, or pharmaceutically acceptable salt thereof, of between about 475 mg and about 525 mg, or about 500 mg, administered daily, and an oral dose of rifampicin or a hydrate, solvate, or pharmaceutically acceptable salt thereof, of between about 575 mg and about 625 mg, or about 590 mg and about 610 mg, or about 600 mg, administered daily. In an exemplary embodiment, the dosage regimen comprises an oral dose of epetraborole or a hydrate, solvate, or pharmaceutically acceptable salt thereof, of between about 475 mg and about 525 mg, or about 500 mg, administered daily, and an oral dose of rifampicin or a hydrate, solvate, or pharmaceutically acceptable salt thereof, of between about 575 mg and about 625 mg, or about 590 mg and about 610 mg, or about 600 mg, administered three days a week.

### V. v.) Epetraborole & Rifabutin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, and rifabutin or a hydrate, solvate, or pharmaceutically acceptable salt thereof, thereby achieving the positive therapeutic response.

In an exemplary embodiment, the epetraborole or a hydrate, solvate, or pharmaceutically acceptable salt thereof, is administered in an oral dose of between about 250 mg and about 1000 mg or about 450 mg and about 550 mg, or about 490 mg and about 510 mg, or about 500 mg, or 500 mg, and the rifabutin is administered in an oral dose of between about 100 mg and about 350 mg, or about 100 and about 200 mg, or about 125 tug and about 175 mg, or about 140 mg and about 160 mg, or about 150 mg or 150 mg, or about 250 mg and about 350 tug, or about 275 mg and about 325 mg, or about 290 tug and about 310 mg, or about 300 mg, or 300 mg.

In an exemplary embodiment, the invention provides a method of achieving a positive therapeutic response against a non-tuberculosis Mycobacteria (NTM) infection comprising administering to a human subject in need of treatment thereof, a dosage regimen comprising: an oral dose amount as described herein of the epetraborole or a hydrate, solvate, or pharmaceutically acceptable salt thereof, administered daily or every other day; and an oral dose amount as described herein of the rifabutin or a hydrate, solvate, or pharmaceutically acceptable salt thereof, administered daily or three days a week.

In an exemplary embodiment, the dosage regimen comprises an oral dose of epetraborole or a hydrate, solvate, or pharmaceutically acceptable salt thereof, of between about 475 tug and about 525 mg, or about 500 mg, administered daily, and an oral dose of rifabutin or a hydrate, solvate, or pharmaceutically acceptable salt thereof, of between about 425 tug and about 475 mg, or about 440 mg and about 460 tug, or about 450 mg, administered daily.

In an exemplary embodiment, the dosage regimen comprises an oral dose of epetraborole or a hydrate, solvate, or pharmaceutically acceptable salt thereof, of between about 475 mg and about 525 mg, or about 500 mg, administered daily, and an oral dose of rifabutin or a hydrate, solvate, or pharmaceutically acceptable salt thereof, of between about 575 mg and about 625 mg, or about 590 mg and about 610 mg, or about 600 mg, administered daily. In an exemplary embodiment, the dosage regimen comprises an oral dose of epetraborole or a hydrate, solvate, or pharmaceutically acceptable salt thereof, of between about 475 mg and about 525 mg, or about 500 mg, administered daily, and an oral dose of rifabutin or a hydrate, solvate, or pharmaceutically acceptable salt thereof, of between about 575 mg and about 625 tug, or about 590 mg and about 610 mg, or about 600 mg, administered three days a week.

### V. vi.) Epetraborole & Ethambutol & Rifampicin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, and rifampicin or a hydrate, solvate, or pharmaceutically acceptable salt thereof, thereby achieving the positive therapeutic response.

In an exemplary embodiment, the epetraborole or a hydrate, solvate, or pharmaceutically acceptable salt thereof, is administered in an oral dose of between about 250 mg and about 1000 tug or about 450 tug and about 550 mg, or about 490 mg and about 510 mg, or about 500 mg, or 500 mg, the ethambutol or a hydrate, solvate, or pharmaceutically acceptable salt thereof, is administered in an oral dose of between about 10 mg/kg and about 30 mg/kg, or about 13 mg/kg or about 17 mg/kg, or about 15 mg/kg, or 15 mg/kg, or about 23 mg/kg or about 27 mg/kg, or about 25 mg/kg, or 25 mg/kg, and the rifampicin is administered in an oral dose of between about 5 mg/kg and about 15 mg/kg, or between about 9 mg/kg or about 11 mg/kg, or about 10 mg/kg, or 10 mg/kg, or between about 350 mg and about 700 mg, or about 350 and about 500 mg, or about 425 mg and about 475 tug, or about 440 mg and about 460 mg, or about 450 mg or 450 mg, or about 500 mg and about 700 mg, or about 550 mg and about 650 mg, or about 575 mg and about 625 mg, or about 590 mg and about 610 mg, or about 600 mg, or 600 mg.

In an exemplary embodiment, the dosage regimen comprises the epetraborole or a hydrate, solvate, or pharmaceutically acceptable salt thereof, administered daily in an oral dose of between about 450 mg and about 550 mg, or about 490 mg and about 510 mg, or about 500 mg, or 500 mg, and the ethambutol or a hydrate, solvate, or pharmaceutically acceptable salt thereof, is administered according to Paragraph Y, and the rifampicin is administered according to Paragraph P.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, and the rifampicin is administered according to Paragraph P.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, and the rifampicin is administered according to Paragraph O.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, and the rifampicin is administered according to Paragraph O.

### V. vii.) Epetraborole & Ethambutol & Rifabutin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, and rifabutin or a hydrate, solvate, or pharmaceutically acceptable salt thereof, thereby achieving the positive therapeutic response.

In an exemplary embodiment, the epetraborole or a hydrate, solvate, or pharmaceutically acceptable salt thereof, is administered in an oral dose of between about 250 mg and about 1000 mg or about 450 mg and about 550 mg, or about 490 mg and about 510 mg, or about 500 mg, or 500 mg, the ethambutol or a hydrate, solvate, or pharmaceutically acceptable salt thereof, is administered in an oral dose of between about 10 mg/kg and about 30 tug/kg, or about 13 tug/kg or about 17 mg/kg, or about 15 mg/kg, or 15 mg/kg, or about 23 mg/kg or about 27 mg/kg, or about 25 mg/kg, or 25 mg/kg, and the rifabutin is administered in an oral dose of between about 100 tug and about 350 mg, or about 100 and about 200 mg, or about 125 mg and about 175 mg, or about 140 mg and about 160 mg, or about 150 mg or 150 mg, or about 250 mg and about 350 mg, or about 275 mg and about 325 mg, or about 290 mg and about 310 mg, or about 300 mg, or 300 mg.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, and the rifabutin is administered according to Paragraph M.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph X, and the rifabutin is administered according to Paragraph M.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, and the rifabutin is administered according to Paragraph L.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, and the rifabutin is administered according to Paragraph L.

V. viii.) Epetraborole & Ethambutol & Azithromycin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, and azithromycin or a hydrate, solvate, or pharmaceutically acceptable salt thereof, thereby achieving the positive therapeutic response.

In an exemplary embodiment, the epetraborole or a hydrate, solvate, or pharmaceutically acceptable salt thereof, is administered in an oral dose of between about 250 mg and about 1000 mg or about 450 mg and about 550 mg, or about 490 mg and about 510 mg, or about 500 mg, or 500 mg, the ethambutol or a hydrate, solvate, or pharmaceutically acceptable salt thereof, is administered in an oral dose of between about 10 mg/kg and about 30 mg/kg, or about 13 mg/kg or about 17 mg/kg, or about 15 mg/kg, or 15 mg/kg, or about 23 mg/kg or about 27 mg/kg, or about 25 mg/kg, or 25 mg/kg, and the azithromycin is administered in an oral dose of between about 200 mg and about 550 mg, or about 200 mg and about 300 mg, or about 225 mg and about 275 mg, or about 250 mg, or about 250 mg, or between about 450 mg and about 550 mg, or about 475 mg and about 525 mg, or about 500 mg, or 500 mg.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, and the azithromycin is administered according to Paragraph V.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, and the azithromycin is administered according to Paragraph V.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, and the azithromycin is administered according to Paragraph U.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, and the azithromycin is administered according to Paragraph U.

### V. iv.) Epetraborole & Ethambutol & Clarithromycin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, and clarithromycin or a hydrate, solvate, or pharmaceutically acceptable salt thereof, thereby achieving the positive therapeutic response.

In an exemplary embodiment, the epetraborole or a hydrate, solvate, or pharmaceutically acceptable salt thereof, is administered in an oral dose of between about 250 mg and about 1000 mg or about 450 mg and about 550 mg, or about 490 mg and about 510 mg, or about 500 mg, or 500 mg, the ethambutol or a hydrate, solvate, or pharmaceutically acceptable salt thereof, is administered in an oral dose of between about 10 mg/kg and about 30 mg/kg, or about 13 mg/kg or about 17 mg/kg, or about 15 mg/kg, or 15 mg/kg, or about 23 mg/kg or about 27 mg/kg, or about 25 mg/kg, or 25 mg/kg, and the clarithromycin is administered in an oral dose of between about 950 mg and about 1,050 mg daily, or about 1,000 daily, or about 400 mg and about 600 mg twice per day, or about 450 mg and about 550 mg twice per day, or about 475 mg and about 525 mg twice per day, or about 500 mg twice per day, or 500 mg twice per day.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, and the clarithromycin is administered according to Paragraph S.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, and the clarithromycin is administered according to Paragraph S.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, and the clarithromycin is administered according to Paragraph R.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, and the clarithromycin is administered according to Paragraph R.

### V. x.) Epetraborole & Ethambutol & Rifampicin & Azithromycin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof rifampicin or a hydrate, solvate, or pharmaceutically acceptable salt thereof, and azithromycin or a hydrate, solvate, or pharmaceutically acceptable salt thereof, thereby achieving the positive therapeutic response.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 tug, or about 500 mg, or 500 mg, the ethambutol or a hydrate, solvate, or pharmaceutically acceptable salt thereof, is administered according to Paragraph Y, the rifampicin is administered according to Paragraph P, and the azithromycin is administered according to Paragraph V.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph Y, the rifampicin is administered according to Paragraph O, and the azithromycin is administered according to Paragraph V.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph P, and the azithromycin is administered according to Paragraph V.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 tug and about 510 mg, or about 500 mg, or 500 mg, the ethambutol or a hydrate, solvate, or pharmaceutically acceptable salt thereof, is administered according to Paragraph X, the rifampicin is administered according to Paragraph O, and the azithromycin is administered according to Paragraph V.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 tug, or about 500 mg, or 500 mg, the ethambutol or a hydrate, solvate, or pharmaceutically acceptable salt thereof, is administered according to Paragraph Y, the rifampicin is administered according to Paragraph P, and the azithromycin is administered according to Paragraph U.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifampicin is administered according to Paragraph O, and the azithromycin is administered according to Paragraph U.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph P, the azithromycin is administered according to Paragraph U.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph X, the rifampicin is administered according to Paragraph O, the azithromycin is administered according to Paragraph U.

### V. xi.) Epetraborole & Ethambutol & Rifampicin & Clarithromycin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifampicin or a hydrate, solvate, or pharmaceutically acceptable salt thereof, and clarithromycin or a hydrate, solvate, or pharmaceutically acceptable salt thereof, thereby achieving the positive therapeutic response.

In an exemplary embodiment, the epetraborole or a hydrate, solvate, or pharmaceutically acceptable salt thereof, is administered in an oral dose of between about 250 mg and about 1000 mg or about 450 mg and about 550 mg, or about 490 mg and about 510 mg, or about 500 mg, or 500 mg, the ethambutol or a hydrate, solvate, or pharmaceutically acceptable salt thereof, is administered in an oral dose of between about 10 mg/kg and about 30 mg/kg, or about 13 mg/kg or about 17 mg/kg, or about 15 mg/kg, or 15 mg/kg, or about 23 Mg/kg or about 27 mg/kg, or about 25 mg/kg, or 25 mg/kg, and the rifampicin is administered in an oral dose of between about 5 mg/kg and about 15 mg/kg, or between about 9 mg/kg or about 11 mg/kg, or about 10 mg/kg, or 10 mg/kg, or between about 350 mg and about 700 mg, or about 350 and about 500 mg, or about 425 mg and about 475 mg, or about 440 mg and about 460 mg, or about 450 mg or 450 mg, or about 500 mg and about 700 mg, or about 550 mg and about 650 mg, or about 575 mg and about 625 mg, or about 590 mg and about 610 mg, or about 600 mg, or 600 mg, and the clarithromycin is administered in an oral dose of between about 950 mg and about 1,050 mg daily, or about 1,000 daily, or about 400 mg and about 600 mg twice per day, or about 450 mg and about 550 mg twice per day, or about 475 mg and about 525 mg twice per day, or about 500 mg twice per day, or 500 mg twice per day.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifampicin is administered according to Paragraph P, and the clarithromycin is administered according to Paragraph S.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifampicin is administered according to Paragraph O, and the clarithromycin is administered according to Paragraph S.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 tug and about 510 mg, or about 500 mg, or 500 mg, the ethambutol or a hydrate, solvate, or pharmaceutically acceptable salt thereof, is administered according to Paragraph X, the rifampicin is administered according to Paragraph P, and the clarithromycin is administered according to Paragraph S.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph O, and the clarithromycin is administered according to Paragraph S.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 tug and about 510 mg, or about 500 mg, or 500 mg, the ethambutol or a hydrate, solvate, or pharmaceutically acceptable salt thereof, is administered according to Paragraph Y, the rifampicin is administered according to Paragraph P, and the clarithromycin is administered according to Paragraph R.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 tug, or about 500 mg, or 500 mg, the ethambutol or a hydrate, solvate, or pharmaceutically acceptable salt thereof, is administered according to Paragraph Y, the rifampicin is administered according to Paragraph O, and the clarithromycin is administered according to Paragraph R.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph P, the clarithromycin is administered according to Paragraph R.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph O, the clarithromycin is administered according to Paragraph R.

### V. xii.) Epetraborole & Ethambutol & Rifabutin & Azithromycin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifabutin or a hydrate, solvate, or pharmaceutically acceptable salt thereof, and azithromycin or a hydrate, solvate, or pharmaceutically acceptable salt thereof, thereby achieving the positive therapeutic response.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph M, and the azithromycin is administered according to Paragraph V.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph L, and the azithromycin is administered according to Paragraph V.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph M, and the azithromycin is administered according to Paragraph V.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph X, the rifabutin is administered according to Paragraph L, and the azithromycin is administered according to Paragraph V.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph M, and the azithromycin is administered according to Paragraph U.

In an exemplary embodiment, the dosage regimen comprises the epetraborole or a hydrate, solvate, or pharmaceutically acceptable salt thereof, is administered daily in an oral dose of between about 450 mg and about 550 mug, or about 490 mg and about 510 tug, or about 500 mg, or 500 mg, the ethambutol or a hydrate, solvate, or pharmaceutically acceptable salt thereof, is administered according to Paragraph Y, the rifabutin is administered according to Paragraph L, and the azithromycin is administered according to Paragraph U.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph M, the azithromycin is administered according to Paragraph U.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph L, the azithromycin is administered according to Paragraph U.

V. xiii.) Epetraborole & Ethambutol & Rifabutin & Clarithromycin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifabutin or a hydrate, solvate, or pharmaceutically acceptable salt thereof, and clarithromycin or a hydrate, solvate, or pharmaceutically acceptable salt thereof, thereby achieving the positive therapeutic response.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph M, and the clarithromycin is administered according to Paragraph S.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph L, and the clarithromycin is administered according to Paragraph S.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph M, and the clarithromycin is administered according to Paragraph S.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph L, and the clarithromycin is administered according to Paragraph S.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph M, and the clarithromycin is administered according to Paragraph R.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph L, and the clarithromycin is administered according to Paragraph R.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph M, the clarithromycin is administered according to Paragraph R.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph L, the clarithromycin is administered according to Paragraph R.

### V. xiv.) Epetraborole & Amikacin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, and amikacin or a hydrate, solvate, or pharmaceutically acceptable salt thereof, thereby achieving the positive therapeutic response. In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, and intravenous amikacin or a hydrate, solvate, or pharmaceutically acceptable salt thereof, thereby achieving the positive therapeutic response. In an exemplary embodiment, the epetraborole or a hydrate, solvate, or pharmaceutically acceptable salt thereof is administered in an oral dose of between about 250 mg and about 1000 mg or about 450 mg and about 550 mg, or about 490 mg and about 510 mg, or about 500 mg, or 500 mg, and the intravenous dose of amikacin is between about 8 mg/kg and about 30 mg/kg, or about 8 mg/kg or about 17 mg/kg, or about 10 mg/kg or about 15 mg/kg, or about 13 mg/kg or about 17 mg/kg, or about 10 mg/kg, or 10 mg/kg, or about 15 mg/kg, or 15 mg/kg, or about 13 mg/kg or about 27 mg/kg, or about 15 mg/kg or about 25 mg/kg, or about 15 mg/kg, or 15 mg/kg, or about 25 mg/kg, or 25 mg/kg.

In an exemplary embodiment, the invention provides a method of achieving a positive therapeutic response against a non-tuberculosis Mycobacteria (NTM) infection comprising administering to a human subject in need of treatment thereof, a dosage regimen comprising: an oral dose amount as described herein of the epetraborole or a hydrate, solvate, or pharmaceutically acceptable salt thereof, administered daily or every other day; and an intravenous dose amount as described herein of the amikacin, administered daily or three days a week.

In an exemplary embodiment, the dosage regimen comprises an oral dose of epetraborole or a hydrate, solvate, or pharmaceutically acceptable salt thereof, of between about 475 mg and about 525 mg, or about 500 mg, administered daily, and an intravenous dose of amikacin, of between about 8 mg/kg or about 17 mg/kg, or about 10 mg/kg or about 15 mg/kg, or about 13 mg/kg or about 17 mg/kg, or about 10 mg/kg, or 10 mg/kg, or about 15 mg/kg, or 15 mg/kg, administered daily.

In an exemplary embodiment, the dosage regimen comprises an oral dose of epetraborole or a hydrate, solvate, or pharmaceutically acceptable salt thereof, of between about 475 mg and about 525 mg, or about 500 mg, administered daily, and an intravenous dose of amikacin, of between about 13 mg/kg or about 27 mg/kg, or about 15 mg/kg or about 25 mg/kg, or about 15 mg/kg, or 15 mg/kg, or about 25 mg/kg, or 25 mg/kg, administered three days a week.

### V. xv.) Epetraborole & Inhaled Amikacin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, and inhaled amikacin or a hydrate, solvate, or pharmaceutically acceptable salt thereof, thereby achieving the positive therapeutic response. In an exemplary embodiment, the epetraborole or a hydrate, solvate, or pharmaceutically acceptable salt thereof, is administered in an oral dose of between about 250 mg and about 1000 mg or about 450 mg and about 550 mg, or about 490 mg and about 510 mg, or about 500 mg, or 500 mg, and the inhaled dose of amikacin is between about 580 mg and about 600 mg, or about 590 mg, or 590 mg.

In an exemplary embodiment, the invention provides a method of achieving a positive therapeutic response against a non-tuberculosis Mycobacteria (NTM) infection comprising administering to a human subject in need of treatment thereof, a dosage regimen comprising: an oral dose amount as described herein of the epetraborole or a hydrate, solvate, or pharmaceutically acceptable salt thereof, administered daily or every other day; and an inhaled dose amount as described herein of the amikacin, administered daily.

In an exemplary embodiment, the dosage regimen comprises an oral dose of epetraborole or a hydrate, solvate, or pharmaceutically acceptable salt thereof, of between about 475 mg and about 525 mg, or about 500 mg, administered daily, and an inhaled dose of amikacin, of between about 580 mg and about 600 mg, or about 590 mg, or 590 mg, administered daily.

### V. xvi.) Epetraborole & Ethambutol & Amikacin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, and amikacin or a hydrate, solvate, or pharmaceutically acceptable salt thereof, thereby achieving the positive therapeutic response. In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, and intravenous amikacin or a hydrate, solvate, or pharmaceutically acceptable salt thereof, thereby achieving the positive therapeutic response.

In an exemplary embodiment, the epetraborole or a hydrate, solvate, or pharmaceutically acceptable salt thereof, is administered in an oral dose of between about 250 mg and about 1000 mg or about 450 mg and about 550 mg, or about 490 mg and about 510 mg, or about 500 mg, or 500 mg, the ethambutol or a hydrate, solvate, or pharmaceutically acceptable salt thereof, is administered in an oral dose of between about 10 mg/kg and about 30 mg/kg, or about 13 mg/kg or about 17 mg/kg, or about 15 mg/kg, or 15 mg/kg, or about 23 mg/kg or about 27 mg/kg, or about 25 mg/kg, or 25 mg/kg, and the amikacin is administered in an intravenous dose of between about 8 mg/kg and about 30 mg/kg, or about 8 mg/kg or about 17 mg/kg, or about 10 mg/kg or about 15 mg/kg, or about 13 mg/kg or about 17 mg/kg, or about 10 mg/kg, or 10 mg/kg, or about 15 mg/kg, or 15 mg/kg, or about 13 mg/kg or about 27 mg/kg, or about 15 mg/kg or about 25 mg/kg, or about 15 mg/kg, or 15 mg/kg, or about 25 mg/kg, or 25 mg/kg.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph Y, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 tug and about 550 mg, or about 490 tug and about 510 mg, or about 500 mg, or 500 mg, the ethambutol or a hydrate, solvate, or pharmaceutically acceptable salt thereof, is administered according to Paragraph X, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, and the amikacin is administered according to Paragraph J.

V. xvii.) Epetraborole & Ethambutol & Inhaled Amikacin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, and inhaled amikacin or a hydrate, solvate, or pharmaceutically acceptable salt thereof, thereby achieving the positive therapeutic response.

In an exemplary embodiment, the epetraborole or a hydrate, solvate, or pharmaceutically acceptable salt thereof, is administered in an oral dose of between about 250 mg and about 1000 mg or about 450 mg and about 550 mg, or about 490 mg and about 510 mg, or about 500 mg, or 500 mg, the ethambutol or a hydrate, solvate, or pharmaceutically acceptable salt thereof, is administered in an oral dose of between about 10 mg/kg and about 30 mg/kg, or about 13 mg/kg or about 17 mg/kg, or about 15 mg/kg, or 15 mg/kg, or about 23 mg/kg or about 27 mg/kg, or about 25 mg/kg, or 25 mg/kg, and the amikacin is administered in an inhaled dose of between about 580 mg and about 600 mg, or about 590 mg, or 590 mg.

In an exemplary embodiment the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, and the amikacin is administered according to Paragraph I.

V. xviii.) Epetraborole & Ciprofloxacin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, and ciprofloxacin or a hydrate, solvate, or pharmaceutically acceptable salt thereof, thereby achieving the positive therapeutic response.

### V. xix.) Epetraborole & Ethambutol & Ciprofloxacin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, and ciprofloxacin or a hydrate, solvate, or pharmaceutically acceptable salt thereof, thereby achieving the positive therapeutic response.

### V. xx.) Epetraborole & Linezolid

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, and linezolid or a hydrate, solvate, or pharmaceutically acceptable salt thereof, thereby achieving the positive therapeutic response.

### V. xxi.) Epetraborole & Ethambutol & Linezolid

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, and linezolid or a hydrate, solvate, or pharmaceutically acceptable salt thereof, thereby achieving the positive therapeutic response.

V. xxii.) Epetraborole & Moxifloxacin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, and moxifloxacin or a hydrate, solvate, or pharmaceutically acceptable salt thereof, thereby achieving the positive therapeutic response.

V. xxiii.) Epetraborole & Ethambutol & Moxifloxacin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, and moxifloxacin or a hydrate, solvate, or pharmaceutically acceptable salt thereof, thereby achieving the positive therapeutic response.

V. xxiv.) Epetraborole & Streptomycin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, and streptomycin or a hydrate, solvate, or pharmaceutically acceptable salt thereof, thereby achieving the positive therapeutic response.

### V. xxv.) Epetraborole & Ethambutol & Streptomycin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, and streptomycin or a hydrate, solvate, or pharmaceutically acceptable salt thereof, thereby achieving the positive therapeutic response.

V. xxvi.) Epetraborole & Tigecycline

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, and tigecycline or a hydrate, solvate, or pharmaceutically acceptable salt thereof, thereby achieving the positive therapeutic response.

V. xxvii.) Epetraborole & Ethambutol & Tigecycline

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, and tigecycline or a hydrate, solvate, or pharmaceutically acceptable salt thereof, thereby achieving the positive therapeutic response.

V. xxviii.) Epetraborole & Ethambutol & Rifampicin & Azithromycin & Amikacin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifampicin or a hydrate, solvate, or pharmaceutically acceptable salt thereof, azithromycin or a hydrate, solvate, or pharmaceutically acceptable salt thereof, and amikacin or a hydrate, solvate, or pharmaceutically acceptable salt thereof, thereby achieving the positive therapeutic response. In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof ethambutol or a hydrate, solvate, or pharmaceutically acceptable salt thereof, rifampicin or a hydrate, solvate, or pharmaceutically acceptable salt thereof, azithromycin or a hydrate, solvate, or pharmaceutically acceptable salt thereof, and intravenous amikacin or a hydrate, solvate, or pharmaceutically acceptable salt thereof, thereby achieving the positive therapeutic response. In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifampicin or a hydrate, solvate, or pharmaceutically acceptable salt thereof, azithromycin or a hydrate, solvate, or pharmaceutically acceptable salt thereof, and inhaled amikacin or a hydrate, solvate, or pharmaceutically acceptable salt thereof, thereby achieving the positive therapeutic response.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph Y, the rifampicin is administered according to Paragraph P, the azithromycin is administered according to Paragraph V, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifampicin is administered according to Paragraph P, the azithromycin is administered according to Paragraph V, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 tug, or about 490 mg and about 510 tug, or about 500 mg, or 500 mg, the ethambutol or a hydrate, solvate, or pharmaceutically acceptable salt thereof, is administered according to Paragraph Y, the rifampicin is administered according to Paragraph P, the azithromycin is administered according to Paragraph V, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 tug and about 510 mg, or about 500 mg, or 500 mg, the ethambutol or a hydrate, solvate, or pharmaceutically acceptable salt thereof, is administered according to Paragraph Y, the rifampicin is administered according to Paragraph O, the azithromycin is administered according to Paragraph V, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifampicin is administered according to Paragraph O, the azithromycin is administered according to Paragraph V, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifampicin is administered according to Paragraph O, the azithromycin is administered according to Paragraph V, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph P, the azithromycin is administered according to Paragraph V, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph X, the rifampicin is administered according to Paragraph P, the azithromycin is administered according to Paragraph V, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph P, the azithromycin is administered according to Paragraph V, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph O, the azithromycin is administered according to Paragraph V, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph O, the azithromycin is administered according to Paragraph V, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph O, the azithromycin is administered according to Paragraph V, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 tug and about 510 mg, or about 500 mg, or 500 mg, the ethambutol or a hydrate, solvate, or pharmaceutically acceptable salt thereof, is administered according to Paragraph Y, the rifampicin is administered according to Paragraph P, the azithromycin is administered according to Paragraph U, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifampicin is administered according to Paragraph P, the azithromycin is administered according to Paragraph U, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph Y, the rifampicin is administered according to Paragraph P, the azithromycin is administered according to Paragraph U, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifampicin is administered according to Paragraph O, the azithromycin is administered according to Paragraph U, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph Y, the rifampicin is administered according to Paragraph O, the azithromycin is administered according to Paragraph U, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifampicin is administered according to Paragraph O, the azithromycin is administered according to Paragraph U, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 tug, or about 500 mg, or 500 mg, the ethambutol or a hydrate, solvate, or pharmaceutically acceptable salt thereof, is administered according to Paragraph X, the rifampicin is administered according to Paragraph P, the azithromycin is administered according to Paragraph U, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph P, the azithromycin is administered according to Paragraph U, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph P, the azithromycin is administered according to Paragraph U, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph O, the azithromycin is administered according to Paragraph U, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph O, the azithromycin is administered according to Paragraph U, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph O, the azithromycin is administered according to Paragraph U, and the amikacin is administered according to Paragraph I.

V. xxix.) Epetraborole & Ethambutol & Rifampicin & Clarithromycin & Amikacin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifampicin or a hydrate, solvate, or pharmaceutically acceptable salt thereof, clarithromycin or a hydrate, solvate, or pharmaceutically acceptable salt thereof, and amikacin or a hydrate, solvate, or pharmaceutically acceptable salt thereof, thereby achieving the positive therapeutic response. In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifampicin or a hydrate, solvate, or pharmaceutically acceptable salt thereof, clarithromycin or a hydrate, solvate, or pharmaceutically acceptable salt thereof, and intravenous amikacin or a hydrate, solvate, or pharmaceutically acceptable salt thereof, thereby achieving the positive therapeutic response. In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifampicin or a hydrate, solvate, or pharmaceutically acceptable salt thereof, clarithromycin or a hydrate, solvate, or pharmaceutically acceptable salt thereof, and inhaled amikacin or a hydrate, solvate, or pharmaceutically acceptable salt thereof, thereby achieving the positive therapeutic response.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifampicin is administered according to Paragraph P, the clarithromycin is administered according to Paragraph S, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifampicin is administered according to Paragraph P, the clarithromycin is administered according to Paragraph S, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifampicin is administered according to Paragraph P, the clarithromycin is administered according to Paragraph S, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifampicin is administered according to Paragraph O, the clarithromycin is administered according to Paragraph S, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifampicin is administered according to Paragraph O, the clarithromycin is administered according to Paragraph S, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 tug, or about 490 mg and about 510 tug, or about 500 mg, or 500 mg, the ethambutol or a hydrate, solvate, or pharmaceutically acceptable salt thereof, is administered according to Paragraph Y, the rifampicin is administered according to Paragraph O, the clarithromycin is administered according to Paragraph S, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph X, the rifampicin is administered according to Paragraph P, the clarithromycin is administered according to Paragraph S, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph P, the clarithromycin is administered according to Paragraph S, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph P, the clarithromycin is administered according to Paragraph S, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph O, the clarithromycin is administered according to Paragraph S, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph X, the rifampicin is administered according to Paragraph O, the clarithromycin is administered according to Paragraph S, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph O, the clarithromycin is administered according to Paragraph S, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 tug, or about 500 mg, or 500 mg, the ethambutol or a hydrate, solvate, or pharmaceutically acceptable salt thereof, is administered according to Paragraph Y, the rifampicin is administered according to Paragraph P, the clarithromycin is administered according to Paragraph R, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifampicin is administered according to Paragraph P, the clarithromycin is administered according to Paragraph R, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifampicin is administered according to Paragraph P, the clarithromycin is administered according to Paragraph R, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph Y, the rifampicin is administered according to Paragraph O, the clarithromycin is administered according to Paragraph R, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifampicin is administered according to Paragraph O, the clarithromycin is administered according to Paragraph R, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifampicin is administered according to Paragraph O, the clarithromycin is administered according to Paragraph R, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph P, the clarithromycin is administered according to Paragraph R, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph P, the clarithromycin is administered according to Paragraph R, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph P, the clarithromycin is administered according to Paragraph R, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph O, the clarithromycin is administered according to Paragraph R, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 tug and about 510 mg, or about 500 mg, or 500 mg, the ethambutol or a hydrate, solvate, or pharmaceutically acceptable salt thereof, is administered according to Paragraph X, the rifampicin is administered according to Paragraph O, the clarithromycin is administered according to Paragraph R, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifampicin is administered according to Paragraph O, the clarithromycin is administered according to Paragraph R, and the amikacin is administered according to Paragraph I.

### V. xxx.) Epetraborole & Ethambutol & Rifabutin & Azithromycin & Amikacin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifabutin or a hydrate, solvate, or pharmaceutically acceptable salt thereof, azithromycin or a hydrate, solvate, or pharmaceutically acceptable salt thereof, and amikacin or a hydrate, solvate, or pharmaceutically acceptable salt thereof, thereby achieving the positive therapeutic response. In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifabutin or a hydrate, solvate, or pharmaceutically acceptable salt thereof, azithromycin or a hydrate, solvate, or pharmaceutically acceptable salt thereof, and intravenous amikacin or a hydrate, solvate, or pharmaceutically acceptable salt thereof, thereby achieving the positive therapeutic response. In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifabutin or a hydrate, solvate, or pharmaceutically acceptable salt thereof azithromycin or a hydrate, solvate, or pharmaceutically acceptable salt thereof, and inhaled amikacin or a hydrate, solvate, or pharmaceutically acceptable salt thereof thereby achieving the positive therapeutic response.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph M, the azithromycin is administered according to Paragraph V, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph M, the azithromycin is administered according to Paragraph V, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph Y, the rifabutin is administered according to Paragraph M, the azithromycin is administered according to Paragraph V, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph L, the azithromycin is administered according to Paragraph V, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph L, the azithromycin is administered according to Paragraph V, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 tug and about 550 mg, or about 490 tug and about 510 mg, or about 500 mg, or 500 mg, the ethambutol or a hydrate, solvate, or pharmaceutically acceptable salt thereof, is administered according to Paragraph Y, the rifabutin is administered according to Paragraph L, the azithromycin is administered according to Paragraph V, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph M, the azithromycin is administered according to Paragraph V, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 tug and about 510 mg, or about 500 mg, or 500 mg, the ethambutol or a hydrate, solvate, or pharmaceutically acceptable salt thereof, is administered according to Paragraph X, the rifabutin is administered according to Paragraph M, the azithromycin is administered according to Paragraph V, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph M, the azithromycin is administered according to Paragraph V, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph L, the azithromycin is administered according to Paragraph V, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph L, the azithromycin is administered according to Paragraph V, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph X, the rifabutin is administered according to Paragraph L, the azithromycin is administered according to Paragraph V, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph M, the azithromycin is administered according to Paragraph U, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph M, the azithromycin is administered according to Paragraph U, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph M, the azithromycin is administered according to Paragraph U, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph L, the azithromycin is administered according to Paragraph U, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph L, the azithromycin is administered according to Paragraph U, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph L, the azithromycin is administered according to Paragraph U, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph M, the azithromycin is administered according to Paragraph U, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 tug, or about 500 mg, or 500 mg, the ethambutol or a hydrate, solvate, or pharmaceutically acceptable salt thereof, is administered according to Paragraph X, the rifabutin is administered according to Paragraph M, the azithromycin is administered according to Paragraph U, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph X, the rifabutin is administered according to Paragraph M, the azithromycin is administered according to Paragraph U, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph L, the azithromycin is administered according to Paragraph U, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph L, the azithromycin is administered according to Paragraph U, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph L, the azithromycin is administered according to Paragraph U, and the amikacin is administered according to Paragraph I.

V. xxxi.) Epetraborole & Ethambutol & Rifabutin & Clarithromycin & Amikacin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifabutin or a hydrate, solvate, or pharmaceutically acceptable salt thereof, clarithromycin or a hydrate, solvate, or pharmaceutically acceptable salt thereof, and amikacin or a hydrate, solvate, or pharmaceutically acceptable salt thereof, thereby achieving the positive therapeutic response. In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifabutin or a hydrate, solvate, or pharmaceutically acceptable salt thereof, clarithromycin or a hydrate, solvate, or pharmaceutically acceptable salt thereof, and intravenous amikacin or a hydrate, solvate, or pharmaceutically acceptable salt thereof, thereby achieving the positive therapeutic response. In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifabutin or a hydrate, solvate, or pharmaceutically acceptable salt thereof, clarithromycin or a hydrate, solvate, or pharmaceutically acceptable salt thereof, and inhaled amikacin or a hydrate, solvate, or pharmaceutically acceptable salt thereof, thereby achieving the positive therapeutic response.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph M, the clarithromycin is administered according to Paragraph S, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 tug and about 510 mg, or about 500 mg, or 500 mg, the ethambutol or a hydrate, solvate, or pharmaceutically acceptable salt thereof, is administered according to Paragraph Y, the rifabutin is administered according to Paragraph M, the clarithromycin is administered according to Paragraph S, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph M, the clarithromycin is administered according to Paragraph S, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph L, the clarithromycin is administered according to Paragraph S, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 tug, or about 500 mg, or 500 mg, the ethambutol or a hydrate, solvate, or pharmaceutically acceptable salt thereof, is administered according to Paragraph Y, the rifabutin is administered according to Paragraph L, the clarithromycin is administered according to Paragraph S, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph Y, the rifabutin is administered according to Paragraph L, the clarithromycin is administered according to Paragraph S, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph M, the clarithromycin is administered according to Paragraph S, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph M, the clarithromycin is administered according to Paragraph S, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph M, the clarithromycin is administered according to Paragraph S, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph X, the rifabutin is administered according to Paragraph L, the clarithromycin is administered according to Paragraph S, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph L, the clarithromycin is administered according to Paragraph S, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 tug, or about 500 mg, or 500 mg, the ethambutol or a hydrate, solvate, or pharmaceutically acceptable salt thereof, is administered according to Paragraph X, the rifabutin is administered according to Paragraph L, the clarithromycin is administered according to Paragraph S, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph M, the clarithromycin is administered according to Paragraph R, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph M, the clarithromycin is administered according to Paragraph R, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph Y, the rifabutin is administered according to Paragraph M, the clarithromycin is administered according to Paragraph R, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph L, the clarithromycin is administered according to Paragraph R, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 tug, or 500 mg, the ethambutol or a hydrate, solvate, or pharmaceutically acceptable salt thereof, is administered according to Paragraph Y, the rifabutin is administered according to Paragraph L, the clarithromycin is administered according to Paragraph R, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph Y, the rifabutin is administered according to Paragraph L, the clarithromycin is administered according to Paragraph R, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph M, the clarithromycin is administered according to Paragraph R, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 tug and about 550 mg, or about 490 tug and about 510 mg, or about 500 mg, or 500 mg, the ethambutol or a hydrate, solvate, or pharmaceutically acceptable salt thereof, is administered according to Paragraph X, the rifabutin is administered according to Paragraph M, the clarithromycin is administered according to Paragraph R, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph M, the clarithromycin is administered according to Paragraph R, and the amikacin is administered according to Paragraph I.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 tug and about 510 mg, or about 500 mg, or 500 mg, the ethambutol or a hydrate, solvate, or pharmaceutically acceptable salt thereof, is administered according to Paragraph X, the rifabutin is administered according to Paragraph L, the clarithromycin is administered according to Paragraph R, and the amikacin is administered according to Paragraph K.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph L, the clarithromycin is administered according to Paragraph R, and the amikacin is administered according to Paragraph J.

In an exemplary embodiment, the dosage regimen comprises the epetraborole or a hydrate, solvate, or pharmaceutically acceptable salt thereof, is administered daily in an oral dose of between about 450 mg and about 550 mg, or about 490 mg and about 510 mg, or about 500 mg, or 500 mg, the ethambutol or a hydrate, solvate, or pharmaceutically acceptable salt thereof, is administered according to Paragraph X, the rifabutin is administered according to Paragraph L, the clarithromycin is administered according to Paragraph R, and the amikacin is administered according to Paragraph I.

V. xxxii.) Epetraborole & Ethambutol & Rifampicin & Azithromycin & Streptomycin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifampicin or a hydrate, solvate, or pharmaceutically acceptable salt thereof, azithromycin or a hydrate, solvate, or pharmaceutically acceptable salt thereof, and streptomycin or a hydrate, solvate, or pharmaceutically acceptable salt thereof, thereby achieving the positive therapeutic response.

V. xxxiii.) Epetraborole & Ethambutol & Rifampicin & Clarithromycin & Streptomycin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifampicin or a hydrate, solvate, or pharmaceutically acceptable salt thereof, clarithromycin or a hydrate, solvate, or pharmaceutically acceptable salt thereof, and streptomycin or a hydrate, solvate, or pharmaceutically acceptable salt thereof, thereby achieving the positive therapeutic response.

V. xxiv.) Epetraborole & Ethambutol & Rifabutin & Azithromycin & Streptomycin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifabutin or a hydrate, solvate, or pharmaceutically acceptable salt thereof, azithromycin or a hydrate, solvate, or pharmaceutically acceptable salt thereof, and streptomycin or a hydrate, solvate, or pharmaceutically acceptable salt thereof, thereby achieving the positive therapeutic response.

V. xxxv.) Epetraborole & Ethambutol & Rifabutin & Clarithromycin & Streptomycin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifabutin or a hydrate, solvate, or pharmaceutically acceptable salt thereof, clarithromycin or a hydrate, solvate, or pharmaceutically acceptable salt thereof, and streptomycin or a hydrate, solvate, or pharmaceutically acceptable salt thereof, thereby achieving the positive therapeutic response.

V. xxxvi.) Epetraborole & Ethambutol & Rifampicin & Moxifloxacin & Amikacin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof rifampicin or a hydrate, solvate, or pharmaceutically acceptable salt thereof, moxifloxacin or a hydrate, solvate, or pharmaceutically acceptable salt thereof, and amikacin or a hydrate, solvate, or pharmaceutically acceptable salt thereof, thereby achieving the positive therapeutic response. In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof ethambutol or a hydrate, solvate, or pharmaceutically acceptable salt thereof, rifampicin or a hydrate, solvate, or pharmaceutically acceptable salt thereof, moxifloxacin or a hydrate, solvate, or pharmaceutically acceptable salt thereof, and intravenous amikacin or a hydrate, solvate, or pharmaceutically acceptable salt thereof, thereby achieving the positive therapeutic response. In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifampicin or a hydrate, solvate, or pharmaceutically acceptable salt thereof, moxifloxacin or a hydrate, solvate, or pharmaceutically acceptable salt thereof, and inhaled amikacin or a hydrate, solvate, or pharmaceutically acceptable salt thereof, thereby achieving the positive therapeutic response.

V. xxxvii.) Epetraborole & Ethambutol & Rifabutin & Moxifloxacin & Amikacin

In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifabutin or a hydrate, solvate, or pharmaceutically acceptable salt thereof, moxifloxacin or a hydrate, solvate, or pharmaceutically acceptable salt thereof, and amikacin or a hydrate, solvate, or pharmaceutically acceptable salt thereof, thereby achieving the positive therapeutic response. In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof ethambutol or a hydrate, solvate, or pharmaceutically acceptable salt thereof, rifabutin or a hydrate, solvate, or pharmaceutically acceptable salt thereof, moxifloxacin or a hydrate, solvate, or pharmaceutically acceptable salt thereof, and intravenous amikacin or a hydrate, solvate, or pharmaceutically acceptable salt thereof, thereby achieving the positive therapeutic response. In an exemplary embodiment, the invention provides a method of achieving a positive therapeutic response against a non-tuberculosis Mycobacteria (NTM) infection comprising administering to a human subject in need of treatment thereof, epetraborole or a hydrate, solvate, or pharmaceutically acceptable salt thereof, ethambutol or a hydrate, solvate, or pharmaceutically acceptable salt thereof, rifabutin or a hydrate, solvate, or pharmaceutically acceptable salt thereof, moxifloxacin or a hydrate, solvate, or pharmaceutically acceptable salt thereof, and inhaled amikacin or a hydrate, solvate, or pharmaceutically acceptable salt thereof, thereby achieving the positive therapeutic response.

### V. a). Non-Tuberculosis Mycobacteria (NTM) Infection

In an exemplary embodiment, the non-tuberculosis Mycobacteria in the infection are rapidly growing. In an exemplary embodiment, the non-tuberculosis Mycobacteria in the infection are M. abscessus, M. arabiense, M. aromaticivorans, M. bacteremicum, M. barrassiae, M. bourgelatii, M. celeriflavum, M. chelonae, M. crocinum, M. fukienense, M. hippocampi, M. insubricum, M. iranicum, M. litorale, M. llatzerense, M. monacense, M. pallens, M. rufum, M. rutilum, M. salmoniphilum, M. sediminis, or Mycobacterium setense, or a combination thereof.

In an exemplary embodiment, the non-tuberculosis Mycobacteria in the infection are slowly growing. In an exemplary embodiment, the non-tuberculosis Mycobacteria in the infection are M. algericum, M. alsiense, M. arosiense, M. bouchedurhonense, M. engbaekii, M. europaeum, M. fragae, M. heraklionense, M. indicus pranii, M. koreense, M. kumamotonense, M. kyorinense, M. lepromatosis, M. liflandii, M. longobardum, M. mantenii, M. marseillense, M. minnesotense, M. noviomagense, M. paraffinicum, M. paragordonae, M. parakoreense, M. paraseoulense, M. paraterrae, M. riyadhense, M. senuense, M. seoulense, M. sherrisii, M. shigaense, M. shinjukuense, M. simulans, M. sinense, M. stomatepiae, M. timonense, M. vulneris, or M. yongonense, or a combination thereof.

In an exemplary embodiment, the non-tuberculosis Mycobacteria in the infection are selected from the group consisting of M. abscessus, M. avium complex (MAC), M. chelonae, M. fortuitum, M. gordonae, M. kansasii, M. mucogenicum, M. peregrinum, and M. xenopi. In an exemplary embodiment, the non-tuberculosis Mycobacteria in the infection are selected from the group consisting of M. abscessus, M. avium complex (MAC), M. fortuitum complex, M. gordonae, M. kansasii, and M. xenopi.

In an exemplary embodiment, the non-tuberculosis Mycobacteria in the infection is Mycobacterium avium complex. In an exemplary embodiment, the non-tuberculosis Mycobacteria in the infection is M. avium, M. intracellulare, M. marseillaise, M. timonense, M. bouchedurhonense, M. colombiense, M. vulneris, or M. chimaera. In an exemplary embodiment, the non-tuberculosis Mycobacteria in the infection is M. intracellulare. In an exemplary embodiment, the non-tuberculosis Mycobacteria in the infection is M. intracellulare subsp. intracellulare or M. intracellulare subsp. In an exemplary embodiment, the non-tuberculosis Mycobacteria in the infection is M. avium.

In an exemplary embodiment, the invention provides a method of achieving a positive therapeutic response against a Mycobacterium avium complex infection as described herein, such as in sections V. i.); V. ii.); V. iii.); V. iv.); V. v.); V. vi.); V. vii.); V. viii.); V. ix.); V. x.); V. xi.); V. xii.); V. xiii.); V. xiv.); V. xv.); V. xvi.); V. xvii.); V. xxiv.); V. xxv.); V. xxviii.); V. xxix.); V. xxx.); V. xxxi.); V. xxxii.); V. xxxiii.); V. xxxiv.); V. xxxv.), thereby achieving the positive therapeutic response. In an exemplary embodiment, the invention provides a composition or method for achieving a positive therapeutic response against a Mycobacterium avium complex infection as described herein, such as in sections III) or III. a.).

In an exemplary embodiment, the invention provides a method of achieving a positive therapeutic response against a Mycobacterium avium infection as described herein, such as in sections V. i.); V. ii.); V. iii.); V. iv.); V. v.); V. vi.); V. vii.); V. viii.); V. ix.); V. x.); V. xi.); V. xii.); V. xiii.); V. xiv.); V. xv.); V. xvi.); V. xvii.); V. xxiv.); V. xxv.); V. xxviii.); V. xxix.) V. xxx.); V. xxxi.); V. xxxii.); V. xxxiii.); V. xxxiv.); V. xxxv.), thereby achieving the positive therapeutic response. In an exemplary embodiment, the invention provides a composition or method for achieving a positive therapeutic response against a Mycobacterium avium complex infection as described herein, such as in sections III) or III. a.).

In an exemplary embodiment, the invention provides a method of achieving a positive therapeutic response against a Mycobacterium intracellulare infection as described herein, such as in sections V. i.); V. ii.); V. iii.); V. iv.); V. v.); V. vi.); V. vii.); V. viii.); V. ix.); V. x.); V. xi.); V. xii.); V. xiii.); V. xiv.); V. xv.); V. xvi.); V. xvii.); V. xxiv.); V. xxv.); V. xxviii.); V. xxix.); V. xxx.); V. xxxi.); V. xxxii.); V. xxxiii.); V. xxxiv.); V. xxxv.), thereby achieving the positive therapeutic response. In an exemplary embodiment, the invention provides a composition or method for achieving a positive therapeutic response against a Mycobacterium avium complex infection as described herein, such as in sections III) or III. a.).

In an exemplary embodiment, the invention provides a method of achieving a positive therapeutic response against Mycobacteria kansasii infection as described herein, such as in sections V. i.); V. ii.); V. iii.); V. iv.); V. v.); V. vi.); V. vii.); V. viii.); V. ix.); V. x.); V. xi.); V. xii.); V. xiii.), thereby achieving the positive therapeutic response. In an exemplary embodiment, the invention provides a composition or method for achieving a positive therapeutic response against a Mycobacterium avium complex infection as described herein, such as in sections III) or III. a.).

In an exemplary embodiment, the invention provides a method of achieving a positive therapeutic response against Mycobacteria kansasii infection as described herein, such as in sections V. i.); V. ii.); V. iii.); V. iv.); V. v.); V. vi.); V. vii.); V. viii.); V. ix.); V. x.); V. xi.); V. xii.); V. xiii.), thereby achieving the positive therapeutic response. In an exemplary embodiment, the invention provides a composition or method for achieving a positive therapeutic response against a Mycobacterium avium complex infection as described herein, such as in sections III) or III a.).

In an exemplary embodiment, the human further has a disease which is cystic fibrosis, chronic obstructive pulmonary disease, or chronic thromboembolic pulmonary hypertension. In an exemplary embodiment, the human further has a disease which is an interstitial lung disease, post-inflammatory lung fibrosis, bronchiectasis, a neoplastic disease, diabetes mellitus, bronchial asthma, hypothyreosis, mediastinal cyst, or rheumatoid arthritis. In an exemplary embodiment, the human further has a disease which is interstitial lung disease, and the interstitial lung disease is idiopathic pulmonary fibrosis, sarcoidosis, or proteinosis. In an exemplary embodiment, the human further has a neoplastic disease, and the neoplastic disease is myelofibrosis or lung cancer. In an exemplary embodiment, the human previously suffered from tuberculosis.

In an exemplary embodiment, the infection is in the lung of the human. In an exemplary embodiment, the infection is in two or more organs in the body. In an exemplary embodiment, the infection is in the lymph nodes.

In an exemplary embodiment, the infection is treatment-naïve, In an exemplary embodiment, the infection is treatment-refractory.

In a further aspect, the non-tuberculosis Mycobacteria infection is part of a non-tuberculosis Mycobacteria-associated disease in the human subject. In an exemplary embodiment, the non-tuberculosis Mycobacteria-associated disease is non-tuberculosis Mycobacteria-pulmonary disease, disseminated non-tuberculosis Mycobacteria disease, or non-tuberculosis Mycobacteria-associated lymphadenitis. In an exemplary embodiment, the non-tuberculosis Mycobacteria-associated disease is Mycobacterium avium complex (MAC) pulmonary disease, disseminated Mycobacterium avium complex (MAC) disease, and Mycobacterium avium complex (MAC)-associated lymphadenitis. In an exemplary embodiment, the non-tuberculosis Mycobacteria-associated disease is nodular bronchiectasis. In an exemplary embodiment, the non-tuberculosis Mycobacteria-associated disease is fibrocavitary.

In an exemplary embodiment, the non-tuberculosis Mycobacteria-associated disease is treatment-naïve. In an exemplary embodiment, the non-tuberculosis Mycobacteria-associated disease is treatment-refractory. In an exemplary embodiment, the non-tuberculosis Mycobacteria-associated disease is treatment-refractory Macrobacterium avium complex (MAC) pulmonary disease. In an exemplary embodiment, the non-tuberculosis Mycobacteria-associated disease is cavitary Mycobacterium avium complex (MAC) pulmonary disease. In an exemplary embodiment, the non-tuberculosis Mycobacteria-associated disease is nodular-bronchiectacic Mycobacterium avium complex (MAC) pulmonary disease.

In an exemplary embodiment, the invention provides a method of achieving a positive therapeutic response against nodular-bronchiectacic Mycobacterium avium complex (MAC) pulmonary disease as described herein, such as in sections V. i.); V. ii.); V. iii.); V. iv.); V. v.); V. vi.); V. vii.); V. viii.); V. ix.); V. x.); V. xi.); V. xii.); V. xiii.), thereby achieving the positive therapeutic response. In an exemplary embodiment, the invention provides a composition or method for achieving a positive therapeutic response against a Mycobacterium avium complex infection as described herein, such as in sections III) or III. a.).

In an exemplary embodiment, the invention provides a method of achieving a positive therapeutic response against cavitary Mycobacterium avium complex (MAC) pulmonary disease as described herein, such as in sections V. i.); V. ii.); V. iii.); V. iv.); V. v.); V. vi.); V. vii.); V. viii.); V. ix.); V. x.); V. xi.); V. xii.); V. xiii.); V. xiv.); V. xvi.); V. xxiv.); V. xxv.); V. xxviii.); V. xxix.); V. xxx.); V. xxxi.); V. xxxii.); and V. xxxiii.), thereby achieving the positive therapeutic response. In an exemplary embodiment, the invention provides a method of achieving a positive therapeutic response against cavitary Mycobacterium avium complex (MAC) pulmonary disease as described herein, such as in sections V. xv.) or V. xvii.), thereby achieving the positive therapeutic response. In an exemplary embodiment, the invention provides a composition or method for achieving a positive therapeutic response against a Mycobacterium avium complex infection as described herein, such as in sections III) or III. a.).

In an exemplary embodiment, the invention provides a method of achieving a positive therapeutic response against treatment-refractory Mycobacterium avium complex (MAC) pulmonary disease as described herein, such as in sections V. i.); V. ii.); V. iii.); V. iv.); V. v.); V. vi.); V. vii.); V. viii.); V. ix.); V. x.); V. xi.); V. xii.); V. xiii.); V. xiv.); V. xv.); V. xvi.); V. xvii.); V. xxiv.); V. xxv.); V. xxviii.); V. xxix.); V. xxx.); V. xxxi.); V. xxxii.); and V. xxxiii.), thereby achieving the positive therapeutic response. In an exemplary embodiment, the invention provides a composition or method for achieving a positive therapeutic response against a Mycobacterium avium complex infection as described herein, such as in sections III) or III. a.).

In an exemplary embodiment, the disease is treated through oral administration of a compound of the invention. In an exemplary embodiment, the disease is treated through oral administration of epetraborole.

### V. b). Assessment of Positive Therapeutic Response

In an exemplary embodiment, the positive therapeutic response can be assessed by assays or tools described herein.

In an exemplary embodiment, prior to administering, an infection marker of the human subject is assessed, thereby obtaining a baseline assessment; after the first administration, the infection marker of the human subject is assessed, thereby obtaining a response assessment, comparing the baseline assessment and the response assessment, and the positive therapeutic response is achieved when the response assessment is an improvement over the baseline assessment. Multiple response assessments can be taken during the course of administering. If later response assessments are an improvement over a previous response assessment, or the baseline assessment, then the positive therapeutic response is achieved.

In an exemplary embodiment, prior to the administration of a dosage regimen described herein, an infection marker of the human subject is assessed, wherein the infection marker, thereby obtaining a baseline assessment; after the first administration of the dosage regimen, the infection marker of the human subject is assessed, thereby obtaining a response assessment, the baseline assessment and the response assessment are compared, and the positive therapeutic response is achieved when the response assessment is an improvement over the baseline assessment. In an exemplary embodiment, the infection marker is assessed within 2 days, or within 3 days, or within 4 days, or within 5 days, or within 6 days, or within 7 days of the conclusion of an occurrence of the dosage regimen. In an exemplary embodiment, the infection marker is assessed within 2 days, or within 3 days, or within 4 days, or within 5 days, or within 6 days, or within 7 days of the conclusion of an occurrence of the dosage regimen as described in Paragraph A. In certain methods, the dosage regimen occurs multiple times. In certain methods, the dosage regimen occurs as described in Paragraph B. In situations where the dosage regimen occurs multiple times, the infection marker of the human subject can be assessed within 2 days, or within 3 days, or within 4 days, or within 5 days, or within 6 days, or within 7 days of the conclusion of an occurrence of the dosage regimen as described in Paragraph A. If later response assessments are an improvement over a previous response assessment, or the baseline assessment, then the positive therapeutic response is achieved.

In an exemplary embodiment, an NTM infection described herein can be assessed for positive therapeutic response(s) by assessing one or more properties of the NTM infection in the patient prior to the administration of the compositions described herein, thereby obtaining a baseline assessment or evaluation. After the first administration of at least one of the dosage regimens described herein, the NTM infection property is assessed or evaluated again, thereby obtaining a response assessment. By comparing the baseline assessment and the response assessment, a determination can be made if the positive therapeutic response has been achieved. If the response assessment is an improvement over the baseline assessment, then the positive therapeutic response against NTM infection is achieved. In an exemplary embodiment, more than one response assessment is made at different times during the course of treatment (during one or more of the times in which the dosage regimen is implemented). In an exemplary embodiment, assessing the NTM infection is done by assessing one or more infection marker(s) of the human subject.

In an exemplary embodiment, the positive therapeutic response can be assessed by quantitative colony count. In an exemplary embodiment, the positive therapeutic response can be assessed by quantitative colony score. In an exemplary embodiment, the positive therapeutic response can be assessed by radiographic analysis.

### V. b.) i.) Quantitative Colony Count

Quantification of bacteria via colony counts on solid agar can be performed according to standard procedures known in the art. An example of such is described in Griffith, et al. Semiquantitative culture analysis during therapy for Mycobacterium avium complex lung disease. Am J Respir Crit Care Med. 2015; 192(6):754-760, where colony counts were predictive of symptomatic and radiographic improvement. As described in Griffith, a quantitative colony count can be obtained from a respiratory sample (such as a sputum sample, such as an expectorated sputum sample or induced sputum sample) obtained from the patient. An induced sputum sample can be obtained from the human subject by, for example, administering a hypertonic saline solution (7% saline, for example) by nebulizer to the human subject. The sputum samples can be processed according to procedures such as decontamination, fluorochrome microscopy, solid medium culture on a biplate of Middlebrook 7H10 agar with and without antibiotics, and a broth culture (BACTEC 960 [Becton Dickinson, Sparks, MD] or ESP [TREK Diagnostic Systems, Cleveland, OH]) as described in Wallace et al, Initial clarithromycin monotherapy for Mycobacterium avium-intracellulare complex lung disease. Am J Respir Crit Care Med 1994; 149:1335-1341; Wallace et al. Clarithromycin regimens for pulmonary Mycobacterium avium complex: the first 50 patients. Am J Respir Crit Care Med 1996; 153:1766-1772. MAC isolates can be identified with, for example, AccuProbe (Hologic-GenProbe, San Diego, CA).

Microbiological evaluation of sputum samples can include assessment in quantitative colony counts. In an exemplary embodiment, a lower quantitative colony count in a later assessment over a previous assessment is a positive therapeutic response. In an exemplary embodiment, a lower quantitative colony count in a response assessment over a baseline assessment is a positive therapeutic response. In an exemplary embodiment, a lower quantitative colony count in a later response assessment over a previous response assessment is a positive therapeutic response. In an exemplary embodiment, a lower quantitative colony count in a second (or third or fourth etc) response assessment over a first response assessment is a positive therapeutic response. In an exemplary embodiment, a lower quantitative colony count in a third (or fourth or fifth etc) response assessment over a second response assessment is a positive therapeutic response. In an exemplary embodiment, a quantitative colony count in the later assessment that is about 1% to about 99% lower than in the previous assessment is a positive therapeutic response. In an exemplary embodiment, a quantitative colony count in the later assessment that is about 1% to about 5% lower, or about 4% to about 9% lower, or about 8% to about 13% lower, or about 12% to about 17% lower, or about 16% to about 21% lower, or about 20% to about 25% lower, or about 24% to about 29% lower, or about 28% to about 33% lower, or about 32% to about 37% lower, or about 36% to about 41% lower, or about 40% to about 45% lower, or about 44% to about 49% lower, or about 48% to about 53% lower, or about 52% to about 57% lower, or about 56% to about 61% lower, or about 60% to about 65% lower, or about 64% to about 69% lower, or about 68% to about 73% lower, or about 72% to about 77% lower, or about 76% to about 81% lower, or about 80% to about 85% lower, or about 84% to about 89% lower, or about 88% to about 93% lower, or about 92% to about 97% lower, or about 96% to about 100% lower, or one or more combinations thereof, than in the previous assessment is a positive therapeutic response.

In an exemplary embodiment, a quantitative colony count in the later assessment that is about 5% to about 25% lower, or about 5% to about 30% lower, or about 5% to about 35% lower, or about 5% to about 40% lower, or about 5% to about 45% lower, or about 5% to about 50% lower, or about 5% to about 55% lower, or about 5% to about 60% lower, or about 5% to about 65% lower, or about 5% to about 70% lower, or about 5% to about 75% lower, or about 5% to about 80% lower, or about 5% to about 85% lower, or about 5% to about 90% lower, or about 5% to about 95% lower, or about 5% to about 99% lower, or about 5% to about 99.2% lower, or about 5% to about 99.5% lower, or about 5% to about 99.7% lower, or about 5% to about 99.9% lower, or about 5% to 100% lower, than in the previous assessment is a positive therapeutic response.

In an exemplary embodiment, a quantitative colony count in the later assessment that is about 10% to about 25% lower, or about 1_0% to about 30% lower, or about 10% to about 35% lower, or about 10% to about 40% lower, or about 10% to about 45% lower, or about 10% to about 50% lower, or about 10% to about 55% lower, or about 10% to about 60% lower, or about 10% to about 65% lower, or about 10% to about 70% lower, or about 10% to about 75% lower, or about 10% to about 80% lower, or about 10% to about 85% lower, or about 10% to about 90% lower, or about 10% to about 95% lower, or about 10% to about 99% lower, or about 10% to about 99.2% lower, or about 10% to about 99.5% lower, or about 10% to about 99.7% lower, or about 10% to about 99.9% lower, or about 10% to 100% lower, than in the previous assessment is a positive therapeutic response.

In an exemplary embodiment, a quantitative colony count in the later assessment that is about 15% to about 25% lower, or about 1_5% to about 30% lower, or about 15% to about 35% lower, or about 15% to about 40% lower, or about 15% to about 45% lower, or about 15% to about 50% lower, or about 15% to about 55% lower, or about 15% to about 60% lower, or about 15% to about 65% lower, or about 15% to about 70% lower, or about 15% to about 75% lower, or about 15% to about 80% lower, or about 15% to about 85% lower, or about 15% to about 90% lower, or about 15% to about 95% lower, or about 15% to about 99% lower, or about 15% to about 99.2% lower, or about 15% to about 99.5% lower, or about 15% to about 99.7% lower, or about 15% to about 99.9% lower, or about 15% to 100% lower, than in the previous assessment is a positive therapeutic response.

In an exemplary embodiment, a quantitative colony count in the later assessment that is about 20% to about 30% lower, or about 20% to about 35% lower, or about 20% to about 40% lower, or about 20% to about 45% lower, or about 20% to about 50% lower, or about 20% to about 55% lower, or about 20% to about 60% lower, or about 20% to about 65% lower, or about 20% to about 70% lower, or about 20% to about 75% lower, or about 20% to about 80% lower, or about 20% to about 85% lower, or about 20% to about 90% lower, or about 20% to about 95% lower, or about 20% to about 99% lower, or about 20% to about 99.2% lower, or about 20% to about 99.5% lower, or about 20% to about 99.7% lower, or about 20% to about 99.9% lower, or about 20% to 100% lower, than in the previous assessment is a positive therapeutic response.

In an exemplary embodiment, prior to the administration of the dosage regimen, an infection marker of the human subject is assessed, wherein the infection marker is quantitative colony count, thereby obtaining a baseline assessment; after the first administration of the epetraborole, the infection marker of the human subject is assessed, thereby obtaining a response assessment, the baseline assessment and the response assessment are compared, and the positive therapeutic response is achieved when the response assessment is an improvement over the baseline assessment.

In an exemplary embodiment, the NTM infection can be assessed for positive therapeutic response(s) by assessing one or more properties of the NTM infection in the patient prior to the administration of the compositions described herein, thereby obtaining a baseline assessment or evaluation. After the first administration of at least one of the compositions described herein, the NTM infection property is assessed or evaluated again, thereby obtaining a response assessment. By comparing the baseline assessment and the response assessment, a determination can be made if the positive therapeutic response has been achieved, if the response assessment is an improvement over the baseline assessment, then the positive therapeutic response against NTM infection is achieved. In an exemplary embodiment, more than one response assessment is made at different times during the course of treatment (during one or more of the times in which the dosage regimen is implemented). In an exemplary embodiment, assessing the NTM infection is done by assessing one or more infection marker(s) of the human subject. In an exemplary embodiment, the infection marker is quantitative colony count.

### Quantitative Colony Score

Microbiological evaluation of sputum samples can include assessment in quantitative colony scores. A categorical score, a quantitative colony score, can be assigned as reported by Griffith, et al. Semiquantitative culture analysis during therapy for Mycobacterium avium complex lung disease. Am J Respir Crit Care Med. 2015; 192(6):754-760. In an exemplary embodiment, a negative culture exhibited no mycobacterial growth, and can be assigned a quantitative colony score of zero; a positive culture growth in broth medium only can be assigned a quantitative colony score of 1; positive growth on solid medium with countable colonies of between 0 and 49 can be assigned a quantitative colony score of 2; positive growth on solid medium with countable colonies of between 50 and 99 can be assigned a quantitative colony score of 3; positive growth on solid medium with countable colonies of between 100 and 199 can be assigned a quantitative colony score of 4; positive growth on solid medium with countable colonies of between 200 and 299 can be assigned a quantitative colony score of 5; positive growth on solid medium with countable colonies of 300 and above can be assigned a quantitative colony score of 6. A human subject who demonstrated no mycobacterial growth (a score of “0”) for three or more consecutive cultures over a minimum of 3 months were labeled as “converters.”

In an exemplary embodiment, a lower quantitative colony score in a later assessment over a previous assessment is a positive therapeutic response. In an exemplary embodiment, a lower quantitative colony score in a response assessment over a baseline assessment is a positive therapeutic response. In an exemplary embodiment, a lower quantitative colony score in a later response assessment over a previous response assessment is a positive therapeutic response. In an exemplary embodiment, a lower quantitative colony score in a second (or third or fourth etc) response assessment over a first response assessment is a positive therapeutic response. In an exemplary embodiment, a lower quantitative colony score in a third (or fourth or fifth etc) response assessment over a second response assessment is a positive therapeutic response. In an exemplary embodiment, a positive therapeutic response is when the quantitative colony score in the later assessment is 1 lower than in the previous assessment. In an exemplary embodiment, a positive therapeutic response is when the quantitative colony score in the later assessment is 1 or more lower than in the previous assessment. In an exemplary embodiment, a positive therapeutic response is when the quantitative colony score in the later assessment is 2 lower than in the previous assessment. In an exemplary embodiment, a positive therapeutic response is when the quantitative colony score in the later assessment is 2 or more lower than in the previous assessment. In an exemplary embodiment, a positive therapeutic response is when the quantitative colony score in the later assessment is 3 lower than in the previous assessment. In an exemplary embodiment, a positive therapeutic response is when the quantitative colony score in the later assessment is 3 or more lower than in the previous assessment. In an exemplary embodiment, a positive therapeutic response is when the quantitative colony score in the later assessment is 4 lower than in the previous assessment. In an exemplary embodiment, a positive therapeutic response is when the quantitative colony score in the later assessment is 4 or more lower than in the previous assessment. In an exemplary embodiment, a positive therapeutic response is when the quantitative colony score in the later assessment is 6 lower than in the previous assessment. In an exemplary embodiment, a positive therapeutic response is when the quantitative colony score in the later assessment is 6 or more lower than in the previous assessment.

In an exemplary embodiment, a quantitative culture score is assessed prior to the first administration of epetraborole. In an exemplary embodiment, a quantitative culture score baseline assessment is taken between 56 days and 1 day prior to the first administration of epetraborole. In an exemplary embodiment, a quantitative culture score baseline assessment is taken the day prior to the first administration of epetraborole. In an exemplary embodiment, a quantitative culture score response assessment is taken after the first administration of epetraborole. In an exemplary embodiment, a quantitative culture score response assessment is taken between about 21 days and about 35 days after the first administration of epetraborole. In an exemplary embodiment, the positive therapeutic response is achieved when the quantitative culture score in the response assessment is at least one or more categorical scores below the quantitative culture score in the baseline assessment. In an exemplary embodiment, the positive therapeutic response is achieved when the quantitative culture score in the response assessment is 1 or 2 or 3 or 4 or 5 or 6 below the quantitative culture score in the baseline assessment. In an exemplary embodiment, the positive therapeutic response is achieved when the quantitative culture score in the response assessment is 1 or 2 below the quantitative culture score in the baseline assessment. In an exemplary embodiment, the positive therapeutic response is achieved when the quantitative culture score in the response assessment is 1 below the quantitative culture score in the baseline assessment. In an exemplary embodiment, the positive therapeutic response is achieved when the quantitative culture score in the response assessment is at least 1 below the quantitative culture score in the baseline assessment.

### Radiographic Assessments

In an exemplary embodiment, the positive therapeutic response is an improvement in a radiographic assessment, such as a chest radiograph or computed tomography (CT) scan of the chest or high-resolution computed tomography (CT) scan of the chest. Such assessments are known in the art, and are described for example in Griffith, et al. Semiquantitative culture analysis during therapy for Mycobacterium avium complex lung disease. Am J Respir Crit Care Med. 2015; 192(6):754-760.

In an exemplary embodiment, chest radiographs (CXR) and high-resolution computed tomography (HRCT) chest scans can be performed prior to the start of a dosage regimen described herein and then at 1- to 2-month intervals for chest radiographs and 6-month intervals for chest CT scans, with more frequent chest CT scans obtained at the discretion of the medical professional/radiologist. In an exemplary embodiment, baseline and response chest radiographs and/or CT scans can be reviewed by the medical professional caring for the patient and a radiologist experienced in reviewing radiographs from NTM patients (such as MAC human subjects with nodular/bronchiectatic disease). In an exemplary embodiment, a second radiologist can review the human subject radiographs. In an exemplary embodiment, the radiographs can be classified as improved (score, 1), no change (score, 0), or worse (score, −1) when compared with the prior radiograph. In an exemplary embodiment, the specific score assignment is based on a global or overall impression of the radiographic appearance. In an exemplary embodiment, the radiograph can have a “mixed” response with some areas of improvement and some areas of increased density compared with the prior radiographic study. In an exemplary embodiment, a discrepancy between the radiologist's and clinician's interpretation of the radiographs can be resolved by the second radiologist blinded to the clinical status of the human subject.

In an exemplary embodiment, a higher radiographic assessment score in a later assessment over a previous assessment is a positive therapeutic response. In an exemplary embodiment, a higher radiographic assessment score in a response assessment over a baseline assessment is a positive therapeutic response. In an exemplary embodiment, a higher radiographic assessment score in a later response assessment over a previous response assessment is a positive therapeutic response. In an exemplary embodiment, a higher radiographic assessment score in a second (or third or fourth etc) response assessment over a first response assessment is a positive therapeutic response. In an exemplary embodiment, a higher radiographic assessment score in a third (or fourth or fifth etc) response assessment over a second response assessment is a positive therapeutic response.

### V. c.) Continuing Administration

In some embodiments, the administering as described herein, such as in Section III or IIIa or V. i)-V. xxxvii.), can continue until the positive therapeutic response is achieved. In some embodiments, the administering as described herein, such as in Section III or IIIa or V. i)-V. xxxvii.), can continue while the positive therapeutic response is present. In some embodiments, the administering as described herein, such as in Section III or IIIa or V. i)-V. xxxvii.), can continue as long as the positive therapeutic response is present. In some embodiments, the administering as described herein, such as in Section III or IIIa or V. i)-V. xxxvii.), can continue for about 6 months, or about 7 months, or about 8 months, or about 9 months, or about 10 months. In some embodiments, the administering as described herein, such as in Section III or IIIa or V. i)-V. xxxvii.), can continue for about 11 months, or about 12 months, or about 13 months, or about 14 months, or about 15 months, or about 16 months. In some embodiments, the administering as described herein, such as in Section III or IIIa or V. i)-V. xxxvii.), can continue for about 16 months, or about 17 months, or about 18 months, or about 19 months, or about 20 months, or about 21 months. In some embodiments, the administering as described herein, such as in Section III or IIIa or V. i)-V. xxxvii.), can continue for about 21 months, or about 22 months, or about 23 months, or about 24 months, or about 25 months, or about 26 months. In some embodiments, the administering as described herein, such as in Section III or IIIa or V. i)-V. xxxvii.), can continue for about 26 months, or about 27 months, or about 28 months, or about 29 months, or about 30 months. In some embodiments, the administering as described herein, such as in Section III or IIIa or V. i)-V. xxxvii.), can continue for at least about 6 months, or about 7 months, or about 8 months, or about 9 months, or about 10 months. In some embodiments, the administering as described herein, such as in Section III or IIIa or V. i) V. xxxvii.), can continue for at least about 11 months, or about 12 months, or about 13 months, or about 14 months, or about 15 months, or about 16 months. In some embodiments, the administering as described herein, such as in Section III or IIIa or V. i)-V. xxxvii.), can continue for at least about 16 months, or about 17 months, or about 18 months, or about 19 months, or about 20 months, or about 21 months. In some embodiments, the administering as described herein, such as in Section III or IIIa or V. i)-V. xxxvii.), can continue for at least about 21 months, or about 22 months, or about 23 months, or about 24 months, or about 25 months, or about 26 months. In some embodiments, the administering as described herein, such as in Section III or IIIa or V. i)-V. xxxvii.), can continue for at least about 26 months, or about 27 months, or about 28 months, or about 29 months, or about 30 months.

In some embodiments, the administering as described herein, such as in Section III or IIIa or V. i)-V. xxxvii.), ends after the positive therapeutic response is no longer present. In some embodiments, the administering as described herein, such as in Section III or Ilia or V. i)-V. xxxvii.), ends at about 6 months, or about 7 months, or about 8 months, or about 9 months, or about 10 months. In some embodiments, the administering as described herein, such as in Section III or IIIa or V. i)-V. xxxvii.), ends at about 11 months, or about 12 months, or about 13 months, or about 14 months, or about 15 months, or about 16 months. In some embodiments, the administering as described herein, such as in Section III or IIIa or V. i)-V. xxxvii.), ends at about 16 months, or about 17 months, or about 18 months, or about 19 months, or about 20 months, or about 21 months. In some embodiments, the administering as described herein, such as in Section III or Ilia or V. i)-V. xxxvii.), ends at about 21 months, or about 22 months, or about 23 months, or about 24 months, or about 25 months, or about 26 months. In some embodiments, the administering as described herein, such as in Section III or Ilia or V. i)-V. xxxvii.), ends at about 26 months, or about 27 months, or about 28 months, or about 29 months, or about 30 months.

### V. d.) Dosage Regimen Occurrences/Repetitions

In some embodiments, the dosage regimen described herein, such as in Sections V. i)-V. xxxvii.), has parameters as described in this section.

PARAGRAPH A: In an exemplary embodiment, the dosage regimen lasts between about 21 and about 35 days. In an exemplary embodiment, the dosage regimen lasts between about 22 and about 34 days. In an exemplary embodiment, the dosage regimen lasts between about 23 and about 33 days. In an exemplary embodiment, the dosage regimen lasts between about 24 and about 32 days. In an exemplary embodiment, the dosage regimen lasts between about 25 and about 31 days. In an exemplary embodiment, the dosage regimen lasts between about 26 and about 30 days. In an exemplary embodiment, the dosage regimen lasts between about 27 and about 29 days. In an exemplary embodiment, the dosage regimen lasts about 28 days. In an exemplary embodiment, the dosage regimen lasts 28 days.

PARAGRAPH B: In an exemplary embodiment, the dosage regimen occurs one time. In an exemplary embodiment, the dosage regimen occurs two times. In an exemplary embodiment, the dosage regimen occurs three times. In an exemplary embodiment, the dosage regimen occurs four times. In an exemplary embodiment, the dosage regimen occurs five times. In an exemplary embodiment, the dosage regimen occurs six times. In an exemplary embodiment, the dosage regimen occurs seven times. In an exemplary embodiment, the dosage regimen occurs eight times. In an exemplary embodiment, the dosage regimen occurs nine times. In an exemplary embodiment, the dosage regimen occurs ten times. In an exemplary embodiment, the dosage regimen occurs eleven times. In an exemplary embodiment, the dosage regimen occurs twelve times. In an exemplary embodiment, the dosage regimen occurs thirteen times. In an exemplary embodiment, the dosage regimen occurs fourteen times. In an exemplary embodiment, the dosage regimen occurs fifteen times. In an exemplary embodiment, the dosage regimen occurs sixteen times. In an exemplary embodiment, the dosage regimen occurs seventeen times. In an exemplary embodiment, the dosage regimen occurs eighteen times. In an exemplary embodiment, the dosage regimen occurs nineteen times. In an exemplary embodiment, the dosage regimen occurs twenty times. In an exemplary embodiment, the dosage regimen occurs twenty-one times. In an exemplary embodiment, the dosage regimen occurs twenty-two times. In an exemplary embodiment, the dosage regimen occurs twenty-three times. In an exemplary embodiment, the dosage regimen occurs twenty-four times. In an exemplary embodiment, the dosage regimen occurs twenty-five times. In an exemplary embodiment, the dosage regimen occurs twenty-six times. In an exemplary embodiment, the dosage regimen occurs twenty-seven times. In an exemplary embodiment, the dosage regimen occurs twenty-eight times. In an exemplary embodiment, the dosage regimen occurs twenty-nine times. In an exemplary embodiment, the dosage regimen occurs thirty times. In an exemplary embodiment, the dosage regimen occurs thirty-one times. In an exemplary embodiment, the dosage regimen repeats until a positive therapeutic response is achieved. In an exemplary embodiment, the dosage regimen continues until a positive therapeutic response is achieved. In an exemplary embodiment, the dosage regimen repeats for as long as there is the positive therapeutic response. In an exemplary embodiment, the dosage regimen continues for as long as there is the positive therapeutic response. In an exemplary embodiment, the dosage regimen is administered to the human subject for as long as there is the positive therapeutic response.

PARAGRAPH C: In an exemplary embodiment, the oral dose of epetraborole is administered once every between about 20 hours and about 28 hours. In an exemplary embodiment, the oral dose of epetraborole is administered once every between about 21 hours and about 27 hours. In an exemplary embodiment, the oral dose of epetraborole is administered once every between about 22 hours and about 26 hours. In an exemplary embodiment, the oral dose of epetraborole is administered once every between about 23 hours and about 25 hours. In an exemplary embodiment, the oral dose of epetraborole is administered once every about 24 hours. In an exemplary embodiment, the oral dose of epetraborole is administered once every 24 hours.

PARAGRAPH D: In an exemplary embodiment, the oral dose of epetraborole is administered once every between about 44 hours and about 52 hours. In an exemplary embodiment, the oral dose of epetraborole is administered once every between about 45 hours and about 51 hours. In an exemplary embodiment, the oral dose of epetraborole is administered once every between about 46 hours and about 50 hours. In an exemplary embodiment, the oral dose of epetraborole is administered once every between about 47 hours and about 49 hours. In an exemplary embodiment, the oral dose of epetraborole is administered once every about 48 hours. In an exemplary embodiment, the oral dose of epetraborole is administered once every 48 hours.

PARAGRAPH E: In an exemplary embodiment, the oral dose of epetraborole is between about 200 mg and about 1100 mg. In an exemplary embodiment, the oral dose of epetraborole is between about 200 mg and about 300 mg. In an exemplary embodiment, the oral dose of epetraborole is between about 225 mg and about 275 mg. In an exemplary embodiment, the oral dose of epetraborole is between about 240 mg and about 260 mg. In an exemplary embodiment, the oral dose of epetraborole is about 250 mg. In an exemplary embodiment, the oral dose of epetraborole is 250 mg. In an exemplary embodiment, the oral dose of epetraborole is between about 400 mg and about 600 mg. In an exemplary embodiment, the oral dose of epetraborole is between about 425 mg and about 575 mg. In an exemplary embodiment, the oral dose of epetraborole is between about 450 mg and about 550 mg. In an exemplary embodiment, the oral dose of epetraborole is between about 475 mg and about 525 mg. In an exemplary embodiment, the oral dose of epetraborole is between about 490 mg and about 510 mg. In an exemplary embodiment, the oral dose of epetraborole is about 500 mg. In an exemplary embodiment, the oral dose of epetraborole is 500 mg. In an exemplary embodiment, the oral dose of epetraborole is between about 650 mg and about 850 mg. In an exemplary embodiment, the oral dose of epetraborole is between about 675 mg and about 825 mg. In an exemplary embodiment, the oral dose of epetraborole is between about 700 mg and about 800 mg. In an exemplary embodiment, the oral dose of epetraborole is between about 725 mg and about 775 mg. In an exemplary embodiment, the oral dose of epetraborole is about 750 mg. In an exemplary embodiment, the oral dose of epetraborole is 750 mg. In an exemplary embodiment, the oral dose of epetraborole is between about 900 mg and about 1100 mg. In an exemplary embodiment, the oral dose of epetraborole is between about 925 mg and about 1075 mg. In an exemplary embodiment, the oral dose of epetraborole is between about 950 mg and about 1050 mg. In an exemplary embodiment, the oral dose of epetraborole is between about 975 mg and about 1025 mg. In an exemplary embodiment, the oral dose of epetraborole is about 1000 mg. In an exemplary embodiment, the oral dose of epetraborole is 1000 mg.

In an exemplary embodiment, the oral dose of epetraborole is about 250 mg, and is administered in one about 250 mg unit dosage form. In an exemplary embodiment, the oral dose of epetraborole is about 500 mg, and is administered in two about 250 mg unit dosage forms. In an exemplary embodiment, the oral dose of epetraborole is about 500 mg, and is administered in one about 500 mg unit dosage form. In an exemplary embodiment, the oral dose of epetraborole is 250 mg, and is administered in one 250 mug unit dosage form. In an exemplary embodiment, the oral dose of epetraborole is 500 mg, and is administered in two 250 mg unit dosage forms. In an exemplary embodiment, the oral dose of epetraborole is 500 mg, and is administered in one 500 mg unit dosage form

In an exemplary embodiment, the oral dose of epetraborole is a tablet. In an exemplary embodiment, the oral dose of epetraborole is a pill. In an exemplary embodiment, the oral dose of epetraborole is a capsule.

In an exemplary embodiment, the oral dose of epetraborole is 250 mg, and is administered in one 250 mg tablet. In an exemplary embodiment, the oral dose of epetraborole is 500 mg, and is administered in two 250 mg tablets. In an exemplary embodiment, the oral dose of epetraborole is 500 mg, and is administered in one 500 mg unit dosage form.

A medical professional having ordinary skill in the art may readily determine and prescribe the effective amount of the oral composition required. For example, a physician could start doses of the medicament employed in the epetraborole composition at levels lower than that required in order to achieve the desired therapeutic effect and gradually increase the dosage until the desired effect is achieved.

Dosage regimens are adjusted to provide the optimum desired response (e.g., a therapeutic response). Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

### V. e). Specific Dosage Regimen Embodiments

In an exemplary embodiment, the invention provides a method of achieving a positive therapeutic response against an infection by non-tuberculosis Macrobacteria (NTM), comprising administering epetraborole to a human subject in need of treatment thereof with a dosage regimen comprising: an oral dose of the epetraborole as described in Paragraph E, administered on the first day of the dosage regimen, and the oral dose is administered as described in Paragraph C, wherein the dosage regimen is as described in Paragraph A and wherein the dosage regimen occurs as described in Paragraph B. In an exemplary embodiment, the invention provides a method of achieving a positive therapeutic response against an infection by non-tuberculosis Mycobacteria (NTM), comprising administering epetraborole to a human subject in need of treatment thereof with a dosage regimen comprising: an oral dose of the epetraborole is between about 240 mg and about 260 mg, administered on the first day of the dosage regimen, and the oral dose is administered as described in Paragraph C, wherein the dosage regimen is as described in Paragraph A and wherein the dosage regimen occurs as described in Paragraph B. In an exemplary embodiment, the invention provides a method of achieving a positive therapeutic response against an infection by non-tuberculosis Mycobacteria (NTM), comprising administering epetraborole to a human subject in need of treatment thereof with a dosage regimen comprising: an oral dose of the epetraborole is between about 475 mg and about 525 mg, administered on the first day of the dosage regimen, and the oral dose is administered as described in Paragraph C, wherein the dosage regimen is as described in Paragraph A and wherein the dosage regimen occurs as described in Paragraph B. In an exemplary embodiment, the invention provides a method of achieving a positive therapeutic response against an infection by non-tuberculosis Mycobacteria (NTM), comprising administering epetraborole to a human subject in need of treatment thereof with a dosage regimen comprising: an oral dose of the epetraborole is between about 725 mg and about 775 mg, administered on the first day of the dosage regimen, and the oral dose is administered as described in Paragraph C, wherein the dosage regimen is as described in Paragraph A and wherein the dosage regimen occurs as described in Paragraph B. In an exemplary embodiment, the invention provides a method of achieving a positive therapeutic response against an infection by non-tuberculosis Mycobacteria (NTM), comprising administering epetraborole to a human subject in need of treatment thereof with a dosage regimen comprising: an oral dose of the epetraborole is between about 1075 mg and about 1125 mg, administered on the first day of the dosage regimen, and the oral dose is administered as described in Paragraph C, wherein the dosage regimen is as described in Paragraph A and wherein the dosage regimen occurs as described in Paragraph B. For any of the embodiments in this paragraph, the oral dose is administered with food. For any of the embodiments in this paragraph, the oral dose is in a fasting condition.

In an exemplary embodiment, the invention provides a method of achieving a positive therapeutic response against an infection by non-tuberculosis Mycobacteria (NTM), comprising administering epetraborole to a human subject in need of treatment thereof with a dosage regimen comprising: an oral dose of the epetraborole as described in Paragraph E, administered on the first day of the dosage regimen, and the oral dose is administered as described in Paragraph D, wherein the dosage regimen is as described in Paragraph A and wherein the dosage regimen occurs as described in Paragraph B. In an exemplary embodiment, the invention provides a method of achieving a positive therapeutic response against an infection by non-tuberculosis Mycobacteria (NTM), comprising administering epetraborole to a human subject in need of treatment thereof with a dosage regimen comprising: an oral dose of the epetraborole is between about 240 mg and about 260 mug, administered on the first day of the dosage regimen, and the oral dose is administered as described in Paragraph D, wherein the dosage regimen is as described in Paragraph A and wherein the dosage regimen occurs as described in Paragraph B. In an exemplary embodiment, the invention provides a method of achieving a positive therapeutic response against an infection by non-tuberculosis Mycobacteria (NTM), comprising administering epetraborole to a human subject in need of treatment thereof with a dosage regimen comprising: an oral dose of the epetraborole is between about 475 mg and about 525 mg, administered on the first day of the dosage regimen, and the oral dose is administered as described in Paragraph D, wherein the dosage regimen is as described in Paragraph A and wherein the dosage regimen occurs as described in Paragraph B. In an exemplary embodiment, the invention provides a method of achieving a positive therapeutic response against an infection by non-tuberculosis Mycobacteria (NTM), comprising administering epetraborole to a human subject in need of treatment thereof with a dosage regimen comprising: an oral dose of the epetraborole is between about 725 mg and about 775 mg, administered on the first day of the dosage regimen, and the oral dose is administered as described in Paragraph D, wherein the dosage regimen is as described in Paragraph A and wherein the dosage regimen occurs as described in Paragraph B. In an exemplary embodiment, the invention provides a method of achieving a positive therapeutic response against an infection by non-tuberculosis Mycobacteria (NTM), comprising administering epetraborole to a human subject in need of treatment thereof with a dosage regimen comprising: an oral dose of the epetraborole is between about 1075 mg and about 1125 mg, administered on the first day of the dosage regimen, and the oral dose is administered as described in Paragraph D, wherein the dosage regimen is as described in Paragraph A and wherein the dosage regimen occurs as described in Paragraph B. For any of the embodiments in this paragraph, the oral dose is administered with food. For any of the embodiments in this paragraph, the oral dose is in a fasting condition.

In an exemplary embodiment, the invention provides a method of achieving a positive therapeutic response against an infection by non-tuberculosis Mycobacteria (NTM), comprising administering epetraborole to a human subject in need of treatment thereof with a dosage regimen comprising: an oral dose of the epetraborole as described in Paragraph E, administered on the first day of the dosage regimen, and the oral dose is administered as described in Paragraph C or Paragraph D, wherein the dosage regimen is as described in Paragraph A and wherein the dosage regimen occurs as described in Paragraph B.

In an exemplary embodiment, the invention provides a method of achieving a positive therapeutic response against an infection by non-tuberculosis Mycobacteria (NTM), comprising administering epetraborole to a human subject in need of treatment thereof with a dosage regimen comprising: an oral dose of the epetraborole between about 475 mg and about 525 mg, administered on the first day of the dosage regimen, and the oral dose is administered as described in Paragraph C, wherein the dosage regimen lasts between about 26 and about 30 days and wherein the dosage regimen occurs as described in Paragraph B. In an exemplary embodiment, the invention provides a method of achieving a positive therapeutic response against an infection by non-tuberculosis Mycobacteria (NTM), comprising administering epetraborole to a human subject in need of treatment thereof with a dosage regimen comprising: an oral dose of the epetraborole between about 475 mg and about 525 mg, administered on the first day of the dosage regimen, and the oral dose is administered as described in Paragraph D, wherein the dosage regimen lasts between about 26 and about 30 days and wherein the dosage regimen occurs as described in Paragraph B.

In an exemplary embodiment, the invention provides a method of achieving a positive therapeutic response against an infection by non-tuberculosis Mycobacteria (NTM), comprising administering epetraborole to a human subject in need of treatment thereof with a dosage regimen comprising: an oral dose of the epetraborole between about 475 mg and about 525 mug, administered on the first day of the dosage regimen, and the oral dose is administered as described in Paragraph C, wherein the dosage regimen lasts about 28 days and wherein the dosage regimen occurs as described in Paragraph B. In an exemplary embodiment, the invention provides a method of achieving a positive therapeutic response against an infection by non-tuberculosis Mycobacteria (NTM), comprising administering epetraborole to a human subject in need of treatment thereof with a dosage regimen comprising: an oral dose of the epetraborole between about 475 mg and about 525 mg, administered on the first day of the dosage regimen, and the oral dose is administered as described in Paragraph D, wherein the dosage regimen lasts about 28 days and wherein the dosage regimen occurs as described in Paragraph B.

In an exemplary embodiment, the invention provides a method of achieving a positive therapeutic response against an infection by non-tuberculosis Mycobacteria (NTM), comprising administering epetraborole to a human subject in need of treatment thereof with a dosage regimen comprising: an oral dose of the epetraborole between about 475 mg and about 525 mg, administered on the first day of the dosage regimen, and the oral dose is administered as described in Paragraph C, wherein the dosage regimen lasts about 28 days and wherein the dosage regimen occurs between 4 and 20 times or between 12 and 17 times. In an exemplary embodiment, the invention provides a method of achieving a positive therapeutic response against an infection by non-tuberculosis Mycobacteria (NTM), comprising administering epetraborole to a human subject in need of treatment thereof with a dosage regimen comprising: an oral dose of the epetraborole between about 475 mg and about 525 mg, administered on the first day of the dosage regimen, and the oral dose is administered as described in Paragraph D, wherein the dosage regimen lasts about 28 days and wherein the dosage regimen wherein the dosage regimen occurs between 4 and 20 times or between 12 and 17 times. In an exemplary embodiment, the invention provides a method of achieving a positive therapeutic response against an infection by non-tuberculosis Mycobacteria (NTM), comprising administering epetraborole to a human subject in need of treatment thereof with a dosage regimen comprising: an oral dose of the epetraborole between about 475 mg and about 525 mg, administered on the first day of the dosage regimen, and the oral dose is administered as described in Paragraph C, wherein the dosage regimen lasts about 28 days and wherein the dosage regimen occurs as long as there is a positive therapeutic response. In an exemplary embodiment, the invention provides a method of achieving a positive therapeutic response against an infection by non-tuberculosis Mycobacteria (NTM), comprising administering epetraborole to a human subject in need of treatment thereof with a dosage regimen comprising: an oral dose of the epetraborole between about 475 mg and about 525 mg, administered on the first day of the dosage regimen, and the oral dose is administered as described in Paragraph D, wherein the dosage regimen lasts about 28 days and wherein the dosage regimen occurs as long as there is a positive therapeutic response. For any of the embodiments in this paragraph, the oral dose is administered with food. For any of the embodiments in this paragraph, the oral dose is in a fasting condition.

In an exemplary embodiment, the invention provides a method of achieving a positive therapeutic response against an infection by non-tuberculosis Mycobacteria (NTM), comprising administering epetraborole to a human subject in need of treatment thereof with an NTM Infection standard of treatment. In an exemplary embodiment, the human subject has been administered at least one previous NTM treatment, which is not the epetraborole wherein the human subject has been administered at least two previous NTM treatments, neither of which is the epetraborole. In an exemplary embodiment, the previous NTM treatment is selected from the group consisting of at least 2 antimycobacterial agents according to the current international treatment guidelines for at least 6 of the last 12 months prior to the first dose of the dosage regimen. In an exemplary embodiment, the previous NTM treatment is selected from the group consisting of clarithromycin, azithromycin, ethambutol, rifampin, rifabutin, amikacin, bedaquiline, clofazimine, linezolid, and a fluoroquinolone. In an exemplary embodiment, the method further comprises administering an optimized background regimen (OBR) treatment to the human subject. In an exemplary embodiment, the optimized background regimen (OBR) treatment is at least 2 antimycobacterial agents according to the current American Thoracic Society (ATS)/European Respiratory Society (ERS)/European Society of Clinical Microbiology and Infectious Diseases (ESCMID)/Infectious Diseases Society of America (IDSA) treatment guidelines.

In an exemplary embodiment, the invention provides a method of achieving a positive therapeutic response against an infection by non-tuberculosis Mycobacteria (NTM), comprising administering according to a dosage regimen described herein, to a human subject in need of treatment thereof, wherein the dosage regimen has a duration as described in Paragraph A and wherein the dosage regimen occurs as described in Paragraph B.

In an exemplary embodiment, the invention provides a method of achieving a positive therapeutic response against an infection by non-tuberculosis Mycobacteria (NTM), comprising administering according to a dosage regimen described in Sections V. i.); V. ii.); V. iii.); V. iv.); V. v.); V. vi.); V. vii.); V. viii); V. ix.); V. x.); V. xi.); V. xii.); V. xiii.); V. xiv.); V. xv.); V. xvi.); V. xvii.); V. xxviii.); V. xxix.); V. xxx.); V. xxxi.), to a human subject in need of treatment thereof, wherein the dosage regimen has a duration as described in Paragraph A and wherein the dosage regimen occurs as described in Paragraph B.

In an exemplary embodiment, the invention provides a method of achieving a positive therapeutic response against an infection by non-tuberculosis Mycobacteria (NTM), comprising administering according to a dosage regimen described in Sections V. i.); V. ii.); V. iii.); V. iv.); V. v); V. vi); V. vii.); V. viii.); V. ix.); V. x.); V. xi.); V. xii.); V. xiii); V. xiv.); V. xv.); V. xvi.); V. xvii.); V. xxviii.); V. xxix.); V. xxx.); V. xxxi.), to a human subject in need of treatment thereof, wherein the dosage regimen has a duration of between about 27 and about 29 days and wherein the dosage regimen occurs as described in Paragraph B.

In an exemplary embodiment, the invention provides a method of achieving a positive therapeutic response against an infection by non-tuberculosis Mycobacteria (NTM), comprising administering according to a dosage regimen described in Sections V. i.); V. ii.); V. iii.); V. iv.); V. v.); to a human subject in need of treatment thereof, wherein the dosage regimen has a duration as described in Paragraph A and wherein the dosage regimen occurs as described in Paragraph B. In an exemplary embodiment, the invention provides a method of achieving a positive therapeutic response against an infection by non-tuberculosis Mycobacteria (NTM), comprising administering according to a dosage regimen described in Sections V. i.); V. ii.); V. iii.); V. iv.); V. v.); to a human subject in need of treatment thereof, wherein the dosage regimen has a duration of between about 27 and about 29 days and wherein the dosage regimen occurs as described in Paragraph B.

In an exemplary embodiment, the invention provides a method of achieving a positive therapeutic response against an infection by non-tuberculosis Mycobacteria (NTM), comprising administering according to a dosage regimen described in Sections V. x.); V. xi.); V. xii.); V. xiii.), to a human subject in need of treatment thereof, wherein the dosage regimen has a duration as described in Paragraph A and wherein the dosage regimen occurs as described in Paragraph B. In an exemplary embodiment, the invention provides a method of achieving a positive therapeutic response against an infection by non-tuberculosis Mycobacteria (NTM), comprising administering according to a dosage regimen described in Sections V. x.); V. xi.); V. xii.); V. xiii.); to a human subject in need of treatment thereof, wherein the dosage regimen has a duration of between about 27 and about 29 days and wherein the dosage regimen occurs as described in Paragraph B.

In an exemplary embodiment, the invention provides a method of achieving a positive therapeutic response against an infection by non-tuberculosis Mycobacteria (NTM), comprising administering according to a dosage regimen described in Sections V. xxviii.), V. xxix.), V. xxx.), and V. xxxi.), to a human subject in need of treatment thereof, wherein the dosage regimen has a duration as described in Paragraph A and wherein the dosage regimen occurs as described in Paragraph B. In an exemplary embodiment, the invention provides a method of achieving a positive therapeutic response against an infection by non-tuberculosis Mycobacteria (NTM), comprising administering according to a dosage regimen described in Sections V. xxviii.), V. xxix.), V. xxx.), and V. xxxi.), to a human subject in need of treatment thereof, wherein the dosage regimen has a duration of between about 27 and about 29 days and wherein the dosage regimen occurs as described in Paragraph B.

### VI. Human Subject Selection

In an exemplary embodiment, the methods described herein can be administered to a human subject having one or more inclusion and/or exclusion criteria described herein. In an exemplary embodiment, the methods described herein can be administered to a human subject having one or more criteria described in Section VI. In an exemplary embodiment, the methods described herein can be administered to a human subject having one or more inclusion criteria described herein. In an exemplary embodiment, the methods described herein can be administered to a human subject having one or more exclusion criteria described herein.

Human subjects can be selected based on criteria described herein. In an exemplary embodiment, the human subject is male. In an exemplary embodiment, the human subject is female. In an exemplary embodiment, the human subject is 18 years or older. In an exemplary embodiment, the human subject is between the ages of 18 years and 100 years. In an exemplary embodiment, the human subject is between the ages of 18 years and 80 years. In an exemplary embodiment, the human subject is between the ages of 45 years and 90 years. In an exemplary embodiment, the human subject is between the ages of 45 years and 80 years. In an exemplary embodiment, the human subject is between the ages of 50 years and 90 years. In an exemplary embodiment, the human subject is between the ages of 55 years and 85 years. In an exemplary embodiment, the human subject is between the ages of 60 years and 80 years. In an exemplary embodiment, the human subject is between the ages of 60 years and 70 years.

In an exemplary embodiment, the human subject has a diagnosis of an NTM infection. In an exemplary embodiment, the human subject has a diagnosis of treatment-refractory NTM infection. In an exemplary embodiment, the human subject has a diagnosis of treatment-refractory NTM disease. In an exemplary embodiment, the human subject has a diagnosis of treatment-refractory NTM lung disease. In an exemplary embodiment, the human subject has a diagnosis of treatment-refractory MAC infection. In an exemplary embodiment, the human subject has a diagnosis of treatment-refractory MAC disease. In an exemplary embodiment, the human subject has a diagnosis of treatment-refractory MAC lung disease. In an exemplary embodiment, the human subject has experienced NTM infection progression. In an exemplary embodiment, the human subject is able to provide written informed consent. In an exemplary embodiment, the human subject has a diagnosis of treatment-refractory MAC lung disease consisting of the following Microbiological criteria: —One Pre-Study MAC-positive respiratory specimen. This specimen can be collected within 6 months prior to Screening, based on documentation in patient medical records. This respiratory specimen can be an expectorated or induced sputum sample, or a deep bronchial sample (eg, bronchoalveolar lavage, bronchial brush, or lung biopsy) collected while the patient was being treated continuously or intermittently with an OBR consisting of at least 2 antimycobacterial agents according to the current ATS/ERS/ESCMID/IDSA treatment guidelines for at least 6 of the last 12 months prior to randomization. If this Pre-Study respiratory specimen was collected >8 weeks to 6 months prior to randomization, the patient may be enrolled in the study only after the Screening sputum sample (below) is reported as MAC-positive by the Central Microbiological Laboratory. In an exemplary embodiment, the human subject has a diagnosis of treatment-refractory MAC lung disease consisting of the following Microbiological criteria: —One Screening MAC-positive expectorated or induced sputum sample. This MAC-positive culture is based on 2 to 3 Screening sputum samples (not deep bronchial specimens) collected within the 8-week Screening Period, which are sent to the Central Microbiological Laboratory for culture and identification (see Laboratory Manual for details). The Screening sputum samples may be collected on separate consecutive days, if needed; at least 1 sputum sample must have a culture that is MAC-positive for inclusion. Antimycobacterial susceptibility results are not required prior to randomization (Note: If the Pre-Study respiratory specimen was collected within 8 weeks prior to randomization, the patient may be enrolled in the study after collection of the Screening sputum sample with a pending (not yet MAC-positive) culture result at the time of randomization at the Investigator's discretion).

In an exemplary embodiment, the human subject is able to comply with all the study activities and procedures throughout the duration of the study, including willingness to adhere to a combination antimycobacterial treatment regimen (including inhaled and parenteral medications, as appropriate, in addition to oral medications) and comply with planned study visits and study procedures (including all planned sputum collections) from Screening through the LFU Visit (study duration up to approximately 20 months, not including Screening). In an exemplary embodiment, the human subject is expected to survive with continued antimycobacterial therapy and appropriate supportive care from Screening through the LFU Visit, in the judgment of the Investigator.

In an exemplary embodiment, the human subject agrees to use an effective method of birth control. In an exemplary embodiment, the human subject is female, and is not pregnant or nursing. In an exemplary embodiment, the female human subject of childbearing potential (FOCPs) either practices sexual abstinence or uses at least 2 medically accepted, effective methods of birth control, which can comprise a combination of a highly effective method (eg, oral contraceptive, indwelling intrauterine device, hormonal implant/patch, injections, or approved cervical ring) and a barrier method (eg, condom, spermicidal gel) from Screening through the EOT Visit and for 90 days following the last dose of epetraborole. Non-childbearing potential is defined as postmenopausal (ie, amenorrheic for at least 1 year) or surgically/naturally sterile. In an exemplary embodiment, the human subject is male, and male patients who are sexually active with a FOCP agree to use an effective barrier method of contraception from Screening through the EOT Visit and for 90 days following the last dose of epetraborole.

In an exemplary embodiment, the human subject does not have predominately fibrocavitary MAC lung disease. In an exemplary embodiment, the human subject does not have a history of treatment-refractory MAC lung disease >5 years. In an exemplary embodiment, the human subject does not have cystic fibrosis or other inherited disorders of airway ciliary dysfunction (eg, primary ciliary dyskinesia). In an exemplary embodiment, the human subject does not have active allergic bronchopulmonary mycosis. In an exemplary embodiment, the human subject does not have anticipated or planned lung surgery for treatment of MAC lung disease. In an exemplary embodiment, the human subject does not have disseminated MAC infection, or other known or suspected non-pulmonary source of infection (eg, infective endocarditis, osteomyelitis, meningitis, or urinary tract infection) requiring non-study antimicrobial therapy. In an exemplary embodiment, the human subject does not have concomitant pulmonary infection requiring antimicrobial therapy, including infection caused by fungi, viruses, non-MAC mycobacteria (eg, Mycobacterium tuberculosis, Myobacterium abscessus, Mycobacterium kansasii), or other bacteria (eg, Pseudomonas aeruginosa, Staphylococcus aureus). In an exemplary embodiment, the human subject does not have active pulmonary malignancy (primary or metastatic) or any malignancy that required or would require chemotherapy or radiation therapy within 1 year prior to randomization through the LFU Visit. In an exemplary embodiment, the human subject does not have creatinine clearance (CrCl) of ≤50 mL/min, as estimated by the Cockcroft-Gault formula, at Screening or at the time of randomization: Estimated CrCl (mL/min)=(140−Age [years])×Actual Body Weight [kg]×[0.85 if Female])/(72×Serum Creatinine [mg/dL]). In an exemplary embodiment, the human subject does not have hemoglobin <10.0 g/dL or <6.2 mmol/L (Grade 2 anemia or worse, based on Common Terminology Criteria for Adverse Events [CTCAE] criteria) at Screening. In an exemplary embodiment, the human subject has not donated blood or plasma within 28 days prior to treatment. In an exemplary embodiment, the human subject does not have symptomatic loss of blood or hemorrhage within 28 days prior to treatment. In an exemplary embodiment, the human subject does not have severe hemoptysis within 28 days prior to treatment, defined as >100 mL (approximately >7 tbsp blood) over any 24-hour period or severe or extremely severe hemoptysis. In an exemplary embodiment, the human subject does not have severe hepatic impairment, as evidenced by alanine aminotransferase (ALT) or aspartate aminotransferase (AST)>3×upper limit of normal (ULN) or total bilirubin >2×ULN, or clinical signs of cirrhosis or end-stage hepatic disease (eg, ascites, hepatic encephalopathy). In an exemplary embodiment, the human subject is not pregnant or breastfeeding. In an exemplary embodiment, the human subject does not have a mean QT interval corrected using Fridericia's formula (QTcF)>480 msec based on triplicate 12-lead ECGs at Screening. In an exemplary embodiment, the human subject does not have an immunodeficiency or an immunocompromised condition and risk for an opportunistic pulmonary infection. In an exemplary embodiment, the human subject does not have a known history of human immunodeficiency virus (HIV) infection plus either an active acquired immunodeficiency syndrome (AIDS)-defining illness in the past 12 months. In an exemplary embodiment, the human subject does not have a known cluster of differentiation 4 (CD4) count <200/mm³ within the past 12 months. In an exemplary embodiment, the human subject does not have neutropenia at time of treatment (absolute neutrophil count <1,000 neutrophils/mm³). In an exemplary embodiment, the human subject has not initiated immunosuppressive therapy within 8 weeks of randomization, including cancer chemotherapy, transplant rejection medication, or chronic use of systemic corticosteroids defined as ≥20 mg/day of prednisone or systemic equivalent. In an exemplary embodiment, the human subject is not anticipated to start a new non-study antimycobacterial therapy during a dosage regimen provided herein. In an exemplary embodiment, the human subject is not receiving any other investigational medication during the 30 days or 5 half-lives, whichever is longer, prior to treatment. In an exemplary embodiment, the human subject has not had prior exposure to epetraborole. In an exemplary embodiment, the human subject does not have any condition that interferes with the ability to safely complete the study or adhere to study requirements, including the patient's inability or unwillingness to comply with all study assessments and visits.

In an exemplary embodiment, the methods described herein cannot be administered to a human subject having undergone a prior therapy as described herein. In an exemplary embodiment, the methods described herein cannot be administered to a human subject having undergone a prohibited therapy as described herein. In an exemplary embodiment, the methods described herein can be administered to a human subject having undergone a concomitant therapy as described herein.

### VII. Miscellaneous

In an exemplary embodiment, the methods described herein can be administered according to an objective described in Example 1. In an exemplary embodiment, a dosing regimen described herein can be administered according to an objective described in Example 1. In an exemplary embodiment, the methods described herein can be administered according to an endpoint described in Example 1. In an exemplary embodiment, a dosing regimen described herein can be administered according to an endpoint described in Example 1.

In an exemplary embodiment, the human subject has been administered at least one previous NTM treatment, which is not the epetraborole. In an exemplary embodiment, the human subject has been administered at least two previous NTM treatments, neither of which is the epetraborole. In an exemplary embodiment, the previous NTM treatment is selected from the group consisting of at least 2 antimycobacterial agents according to the current international treatment guidelines for at least 6 of the last 12 months prior to the first dose of the dosage regimen. In an exemplary embodiment, the previous NTM treatment is selected from the group consisting of clarithromycin, azithromycin, ethambutol, rifampin, rifabutin, amikacin, bedaquiline, clofazimine, linezolid, and a fluoroquinolone. In an exemplary embodiment, the method further comprising administering an optimized background regimen (OBR) treatment to the human subject. In an exemplary embodiment, the optimized background regimen (OBR) treatment is at least 2 antimycobacterial agents according to the current American Thoracic Society (ATS)/European Respiratory Society (ERS)/European Society of Clinical Microbiology and Infectious Diseases (ESCMID)/Infectious Diseases Society of America (IDSA) treatment guidelines.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification controls.

### EXAMPLES

Examples are provided below to illustrate the present invention. These examples are not meant to constrain the present invention to any particular application or theory of operation.

General and specific scientific techniques are outlined in U.S. Pat. No. 7,816,344, as well as PCT Publication WO2008/157726 A1, each of which are expressly incorporated by reference in their entirety and particularly for the techniques outlined therein.

### Example 1

### Treatment Plan

### Phase 2 Objectives and Endpoints

A primary objective of Phase 2 is to assess the microbiological response to oral epetraborole compared to placebo. Another primary objective of Phase 2 is to assess the safety of oral epetraborole compared to placebo. A secondary objective of Phase 2 is to evaluate the plasma PK of oral epetraborole; the dosage of epetraborole can be confirmed based on a PK analysis from approximately 16 epetraborole patients.

An exploratory objective of Phase 2 is to assess clinical and microbiological responses in subgroups (eg, age, amikacin liposome inhalation suspension [ALIS] use at baseline [pre-randomization], MAC resistance phenotype). Another exploratory objective of Phase 2 is to assess pulmonary radiographic response. Another exploratory objective of Phase 2 is to assess microbiological response by epetraborole MIC. Another exploratory objective of Phase 2 is to evaluate postbaseline MAC isolates for decreased susceptibility to epetraborole compared to baseline. Another exploratory objective of Phase 2 is to assess time to microbiological response. Another exploratory objective of Phase 2 is to assess microbiological reinfection and relapse.

A primary endpoint of Phase 2 is by-subject sputum conversion monthly through Month 6. Sputum conversion can be assessed using culture conversion based on 3 consecutive monthly negative sputum cultures for MAC. Another primary endpoint of Phase 2 is the assessment of TEAEs and changes from baseline in clinical laboratory values, electrocardiograms (ECGs), and vital sign changes.

A secondary endpoint of Phase 2 is by-subject microbiological improvement monthly through Month 6. Microbiological improvement can be assessed using decrease in MAC colony counts of ≥1 category as described herein. Another secondary endpoint is the Characterization of PK parameters (eg, Vd, Cₘₐₓ, AUC) in epetraborole recipients.

An exploratory endpoint for Phase 2 is clinical response and microbiological response (by-subject sputum culture conversion and by-subject microbiological improvement) at Month 3 and Month 6 presented by age category, ALIS use at baseline, and MAC resistance phenotype (ie, macrolide-resistant and amikacin-resistant). Another exploratory endpoint for Phase 2 is radiographic response at Month 6 and End of Therapy (EOT), defined as overall change from baseline based on blinded central reading of chest computed tomography (CT). An exploratory endpoint for Phase 2 is by-subject microbiological response (by-subject sputum culture conversion and by-subject microbiological improvement) by epetraborole MIC at Month 6. Another exploratory endpoint for Phase 2 is the rate of decreased susceptibility among postbaseline MAC isolates, defined as a 4-fold increase in epetraborole MIC relative to the baseline isolate MIC, among patients remaining MAC culture-positive at Month 4 or later. Another exploratory endpoint for Phase 2 is time (in months) to first negative sputum culture and decrease in MAC colony counts by ≥1 category. Another exploratory endpoint for Phase 2 is rates of reinfection (new pulmonary MAC infection caused by pathogen[s] different from the baseline MAC isolate as determined by molecular typing) and relapse (pulmonary MAC infection caused by the same baseline MAC isolate as determined by molecular typing) at Month 6.

### Phase 3 Objectives and Endpoints

A primary objective of Phase 3 is to determine if epetraborole+OBR is superior to placebo+OBR in microbiological response. A secondary objective of Phase 3 is to assess the safety of oral epetraborole compared to placebo. Another secondary objective of Phase 3 is to assess the plasma PK of oral epetraborole. An exploratory objective of Phase 3 is to assess clinical and microbiological responses in subgroups (eg, age, ALIS use at baseline, MAC resistance phenotype). Another exploratory objective of Phase 3 is to assess pulmonary radiographic response. Another exploratory objective of Phase 3 is to assess microbiological response by epetraborole MIC. Another exploratory objective of Phase 3 is to evaluate postbaseline MAC isolates for decreased susceptibility to epetraborole compared to baseline. Another exploratory objective of Phase 3 is to assess time to microbiological response. Another exploratory objective of Phase 3 is to assess microbiological reinfection and relapse.

A primary endpoint for Phase 3 is by-subject sputum conversion based on 3 consecutive monthly negative sputum cultures for MAC by Month 6. A secondary endpoint for Phase 3 is by-subject microbiological improvement at Month 3 and Month 6 using decrease in MAC colony counts of ≥1 category. Another secondary endpoint for Phase 3 is assessment of TEAEs and changes from baseline in clinical laboratory values, ECGs, and vital sign changes. Another secondary endpoint for Phase 3 is characterization of PK parameters (eg, Vd, Cₘₐₓ, AUC) in epetraborole recipients. An exploratory endpoint for Phase 3 is clinical response and microbiological response (by-subject sputum culture conversion and by-subject microbiological improvement) at Month 3 and Month 6 presented by age category, ALIS use at baseline, and MAC resistance phenotype (macrolide-resistant and amikacin-resistant). Another exploratory endpoint for Phase 3 is radiographic response at Month 6 and LOT, defined as overall change from baseline based on blinded central reading of chest CT. Another exploratory endpoint for Phase 3 is by-subject microbiological response (by-subject sputum culture conversion and by-subject microbiological improvement) by epetraborole MIC at Month 6. Another exploratory endpoint for Phase 3 is rate of decreased susceptibility among postbaseline MAC isolates, defined as a ≥4-fold increase in epetraborole MIC relative to the baseline isolate MIC, among patients remaining MAC culture-positive at Month 4 or later. Another exploratory endpoint for Phase 3 is time (in months) to first negative sputum culture and decrease in MAC colony counts by ≥1 category. Another exploratory endpoint for Phase 3 is rates of reinfection (new pulmonary MAC infection caused by strain[s] different from the baseline MAC isolate as determined by molecular typing) and relapse (pulmonary MAC infection caused by the same baseline MAC isolate as determined by molecular typing) at Month 6, EOT, and Late Follow-up (LFU).

### Study Design

This is a Phase 2/3, double-blind, placebo-controlled study of epetraborole+OBR (Optimized Background Regimen) versus placebo+OBR in patients with treatment-refractory MAC lung disease. This study can enroll adult patients with treatment-refractory MAC lung disease who meet all eligibility criteria. In addition to the clinical criteria required to meet the disease definition, the microbiological criteria define “treatment-refractory” MAC lung disease as respiratory specimen culture positivity for MAC while the patient is being treated continuously or intermittently with an OBR consisting of at least 2 antimycobacterial agents according to the current international treatment guidelines for at least 6 of the last 12 months prior to) randomization. Randomization can be stratified by baseline use of Amikacin Liposome Inhalation Suspension (ALIS) and age at informed consent (<65 years versus ≥65 years).

This study features a sequential Phase 2/3 approach with an initial Phase 2 assessment of symptom-based clinical responses, microbiological responses, safety, and PK associated with oral epetraborole, prior to the superiority analysis of oral epetraborole versus placebo in the Phase 3 part of the study.

In the Phase 2 part of the study, approximately 80 patients can be randomized in a 1:1 ratio (40 patients receiving active epetraborole tablets and 40 patients receiving matching placebo tablets) using an Interactive Responsive Technology (IRT) system.

After the last patient in Phase 2 completes the Month 6 visit, the Sponsor can be unblinded for Phase 2 data analyses. Patients, Investigators, and other study staff can also remain blinded to treatment assignment through the last study visit (LFU). Phase 2 data analyses can include review of microbiological, safety, and PK data collected at multiple time points through Month 6. The Phase 3 part of the study can start enrolling after the last patient in the Phase 3 dose is confirmed upon review of PK data from Phase 2. Patients enrolled in the Phase 2 part of the study are not eligible for participation in Phase 3. Patients in Phase 2 can continue taking blinded study drug for 12 months after the first negative MAC culture that defines sputum culture conversion; study drug can be discontinued in patients who remain MAC culture-positive despite 6 months of therapy with study drug (based on Month 4 sputum culture results).

The following figure presents the study design.

The Phase 3 part of the study can test the superiority of epetraborole+OBR compared to placebo+OBR. In this part of the study, approximately 234 patients are planned to be randomized in a 2:1 ratio (156 patients receiving active epetraborole tablets and 78 patients receiving matching placebo tablets) using an IRT system. In addition, the Sponsor can determine if the sample size for Phase 3 should be adjusted and will verify the Phase 3 epetraborole dosage regimen based on the observed plasma epetraborole exposure. After the last patient in Phase 3 completes the Month 6 visit, the Sponsor can be unblinded for Phase 3 data analyses; patients, Investigators, and other study staff can remain blinded to treatment assignment through the last study visit (LFU). Phase 3 data analyses will include a review of patient-reported clinical symptom responses, microbiological, safety, and PK data collected at multiple time points through Month 6. Patients in Phase 3 can continue double-blinded study drug for 12 months after the first negative MAC culture that defines sputum culture conversion; study drug can be discontinued in patients who remain MAC culture-positive despite 6 months of therapy with study drug (based on Month 4 sputum culture results).

During the conduct of the study, an independent Data and Safety Monitoring Board (DSMB) can be responsible for periodic review of unblinded study safety data by performing a qualitative and quantitative safety assessment. In addition, the DSMB can determine whether the basic study assumptions remain valid, and evaluate whether the overall integrity, scientific merit, and conduct of the study remain acceptable. The DSMB can make recommendations to the Sponsor regarding continuation or termination of the study or suggested changes in the study design/procedures.

### Study Assessments and Procedures

Study assessments and procedures can be completed at the following visits for both Phase 2 and Phase 3 parts of the study:

Screening Visit: Screening assessments to determine study eligibility can be performed within 8 weeks prior to randomization. Two to 3 sputum samples can be collected during screening and may be collected on separate consecutive days. The Screening sputum samples can be sent to the Central Microbiological Laboratory (National Jewish Lab) for culture, identification, quantification, susceptibility testing, and molecular testing. If the pre-study MAC-positive respiratory specimen was collected within 8 weeks prior to randomization, the patient may be enrolled in the study after collection of the screening sputum sample and a pending (not yet MAC-positive) screening culture result. Antimycobacterial susceptibility results are not required prior to randomization. If this pre-study MAC-positive respiratory specimen was collected >8 weeks to 6 months prior to randomization, the patient may be enrolled in the study after the screening sputum sample is reported as MAC-positive by the Central Microbiological Laboratory (only identification to MAC level rather than species level is required for randomization).

Note: Patients with screening sputum sample cultures that are finalized as MAC-negative after randomization can be prematurely discontinued from study drug at the time that the final screening pending culture is reported as negative, and their OBR can be continued; however, they can remain in the study (ie, they will not be withdrawn from the study) to undergo all scheduled EOT and LFU assessments, including safety assessments.

Treatment Period: Patients can receive blinded study drug (epetraborole or placebo) from Day 1 to EOT. EOT can be up to 12 months from the time of the first sputum culture conversion to a negative MAC culture for patients who were culture-converters by Month 6, or at the time of premature discontinuation of study drug for any reason. Study drug can be discontinued in patients who remain MAC culture-positive despite 6 months of therapy with study drug (based on Month 4 sputum culture results). Reporting of final respiratory culture and identification results may occur up to 8 weeks after specimen receipt at the central laboratory.

During the first 6 months of the treatment period, study assessments (including microbiological, clinical, safety, and PK assessments) can be performed from Day 1 (calendar day of first dose of study drug) and every 28 days (±7 days) thereafter, up to Month 6 (Day 169±7 days).

Patients who culture-convert based on 3 consecutive monthly negative sputum cultures for MAC by Month 6 (Day 169±7 days) can continue blinded study drug (the Investigator, study site, and patients will remain blinded) for 12 months from the first month that defines sputum culture conversion up to a maximum of 16 months in total.

For patients continuing blinded study drug during the Treatment Period after Month 6, study assessments (including microbiological, clinical, safety, and PK assessments) can be performed every 3 months (84 days±7 days) thereafter, up to EOT (up to 12 months from the time of the first sputum culture conversion to a negative MAC culture).

EOT Visit: The EOT Visit can be performed within 7 calendar days after the last dose of study drug. Patients requiring more than the maximum 16 months of MAC lung disease treatment for any reason can be discontinued from study drug and treated with an appropriate open-label antimycobacterial regimen. EOT can occur up to 12 months after the first negative respiratory culture for MAC (ie, the first month that defines sputum MAC culture conversion), or at the time of premature discontinuation of study drug for any reason, including continued positive cultures despite 6 months of study drug therapy. For patients who are withdrawn from the study prior to completion, the EOT Visit procedures can be performed at an Early Termination Visit. The EOT assessments may be performed during a regularly scheduled clinic visit if within the EOT window.

LFU Visit: The LFU Visit can be performed 3 months (84 days±14 days) after the last dose of study drug.

Patients who are prematurely discontinued from study drug for any reason can remain in the study (ie, will not be withdrawn from the study) to undergo all scheduled EOT and LFU assessments, including safety assessments.

### Duration of Participation

For the Phase 2 and Phase 3 parts of the study, the duration of study participation for each patient, not including Screening, can be up to approximately 19 months.

Each patient's duration of study participation varies depending on the timing of microbiological sputum MAC culture conversion. To receive study drug beyond Month 6, a subject can achieve a microbiological sputum MAC culture conversion based on 3 consecutive monthly negative sputum cultures by Month 6. Patients can continue taking blinded study drug for 12 months after the first month that defines sputum culture conversion, up to a maximum of 16 months. Study drug can be discontinued in patients who remain MAC culture-positive despite 6 months of therapy with study drug (based on Month 4 sputum culture results).

For example, a subject who has 3 consecutive negative results at Months 4, 5, and 6, can continue with 12 additional months of blinded study drug from Month 4 (the first month of conversion) to Month 16. This subject's EOT Visit would be at Month 16 (ie, 12 months after Month 4) and the LFU Visit would be at Month 19 (ie, 3 months [84 days±14 days] after last dose).

### Inclusion Criteria

Inclusion Criteria for Subjects of the Study include: 1. Male or female patients who are 18 years of age or older. 2. Willing and able to provide written informed consent. 3. Patients with a diagnosis of treatment-refractory MAC lung disease consisting of the following Microbiological and/or Radiographic Criteria: Microbiological criteria: —One pre-study MAC-positive respiratory specimen. In an exemplary embodiment, this specimen is collected within 6 months prior to Screening, based on documentation in patient medical records. In an exemplary embodiment, documentation is provided of a MAC-positive specimen collected per standard of care within 6 months prior to signing the Informed Consent Form (ICF). In an exemplary embodiment, this respiratory specimen is an expectorated or induced sputum sample, or a deep bronchial sample (eg, bronchoalveolar lavage, bronchial brush, or lung biopsy) collected while the patient was being treated continuously or intermittently with an OBR consisting of at least 2 antimycobacterial agents according to the current American Thoracic Society (ATS)/European Respiratory Society (ERS)/European Society of Clinical Microbiology and Infectious Diseases (ESCMID)/Infectious Diseases Society of America (IDSA) treatment guidelines for at least 6 of the last 12 months prior to randomization. In an exemplary embodiment, this Pre-Study is collected during continuous or intermittent treatment with an OBR consisting of at least 2 antimycobacterial agents according to the current American Thoracic Society (ATS)/European Respiratory Society (ERS)/European Society of Clinical Microbiology and Infectious Diseases (ESCMID)/Infectious Diseases Society of America (IDSA) treatment guidelines for at least 6 of the last 12 months prior to randomization. In an exemplary embodiment, if this Pre-Study respiratory specimen was collected >8 weeks to 6 months prior to randomization, the patient may be enrolled in the study only after the Screening sputum sample is reported as MAC-positive by the Central Microbiological Laboratory. In an exemplary embodiment, the Pre-Study respiratory specimen may be an expectorated or induced sputum sample, or a deep bronchial sample (eg, bronchoalveolar lavage, bronchial brush, or lung biopsy). —One Screening MAC-positive expectorated or induced sputum sample. In an exemplary embodiment, the MAC-positive culture is based on 2 to 3 Screening sputum samples (not deep bronchial specimens) collected within the 8-week screening period, which are sent to the Central Microbiological Laboratory for culture and identification. In an exemplary embodiment, the MAC-positive culture is based on 2 to 3 Screening sputum samples (not deep bronchial specimens) collected within the 8-week screening period (after the ICF is signed), which are sent to the Central Microbiological Laboratory for culture and identification. In an exemplary embodiment, the Screening sputum samples may be collected on separate consecutive days, if needed; at least 1 sputum sample must have a culture that is MAC-positive for inclusion. In an exemplary embodiment, the Screening sputum samples may be collected on the same day or separate consecutive days. Antimycobacterial susceptibility results are not required prior to randomization. If the Pre-Study MAC-positive respiratory specimen was collected >8 weeks to 6 months prior to randomization, the patient may be enrolled in the study only after the Screening sputum sample is reported as MAC-positive by the central microbiological laboratory. If the Pre-Study MAC-positive respiratory specimen was collected within 8 weeks prior to randomization, the patient may be enrolled (at the Investigator's discretion) after collection of the Screening sputum sample and a pending (not yet MAC-positive) Screening culture result. Radiographic criteria: Chest CT scan within 8 weeks prior to randomization with abnormalities consistent with MAC lung disease. Vote: MAC lung disease with radiographic evidence of cavities is allowed, provided the maximum diameter of the largest cavity on chest CT is 13.0 cm in diameter. Patients with predominately fibrocavitary MAC lung disease or the presence of any cavity >3.0 cm in diameter are excluded. 4. Patients who are willing to comply with all the study activities and procedures throughout the duration of the study, including willingness to adhere to a combination antimycobacterial treatment regimen (including inhaled and parenteral medications, as appropriate, in addition to oral medications) and comply with all planned study visits and study procedures (including all planned sputum collections) from Screening through the LFU Visit (study duration up to approximately 19 months, not including Screening). 5. All patients must agree to use an effective method of birth control. a. Female patients must not be pregnant or nursing. Female patients of childbearing potential (FOCPs) must commit to either sexual abstinence or use of at least 2 medically accepted, effective methods of birth control, which can comprise of a combination of a highly effective method (eg, oral contraceptive, indwelling intrauterine device, hormonal implant/patch, injections, or approved cervical ring) and a barrier method (eg, condom, spermicidal gel) from Screening through the EOT Visit and for 90 days following the last dose of study drug. Non-childbearing potential is defined as postmenopausal (ie, amenorrheic for at least 1 year) or surgically/naturally sterile. b. Male patients who are sexually active with a FOCP must agree to use an effective barrier method of contraception from Screening through the EOT Visit and for 90 days following the last dose of study drug. 6. Patients expected to survive with continued antimycobacterial therapy and appropriate supportive care from Screening through the LFU Visit.

### Exclusion Criteria

Exclusion Criteria for Subjects of the Study include: 1. Patients with a presence of any suspected or confirmed disease or condition at Screening or the time of randomization that, in the opinion of the Investigator, may confound the assessment of symptom-based clinical response, including, but not limited to, the following: Predominately fibrocavitary MAC lung disease or radiographic presence of any cavity >3.0 cm in diameter; History of treatment-refractory MAC lung disease >5 years; Cystic fibrosis or other inherited disorders of airway ciliary dysfunction (eg, primary ciliary dyskinesia); Active allergic bronchopulmonary mycosis; Anticipated or planned lung surgery for treatment of MAC lung disease; Disseminated MAC infection, or other known or suspected non-pulmonary source of infection (eg, infective endocarditis, osteomyelitis, meningitis, or urinary tract infection) requiring non-study antimicrobial therapy; Concomitant pulmonary infection requiring antimicrobial therapy, including infection caused by fungi, viruses, non-MAC mycobacteria (eg, Mycobacterium tuberculosis, Mycobacterium abscessus, Mycobacteriumr kansasii), or other bacteria (eg, Pseudomonas aeruginosa, Staphylococcus aureus); Note: Patients with MAC lung disease and concomitant non-MAC lung infection requiring antimicrobial therapy can complete the antimicrobial treatment prior to randomization. Patients with respiratory specimen cultures that contain growth of non-MAC organisms that are deemed by the Investigator to be respiratory tract colonizers and who do not require or receive specific antimicrobial therapy may remain eligible. 2. Patients with active pulmonary malignancy (primary or metastatic) or any malignancy that required or would require chemotherapy or radiation therapy within 1 year prior to randomization through the LFU Visit. 3. Patients with creatinine clearance (CrCl) of ≤50 mL/min, as estimated by the Cockcroft-Gault formula, at Screening or at the time of randomization: Estimated CrCl (mL/min)=(140−Age [years])×Actual Body Weight [kg]×[0.85 if Female])/(72×Serum Creatinine [mg/dL]). 4. Patients with hemoglobin <10.0 g/dL or <6.2 mmol/L (Grade 2 anemia or worse, based on Common Terminology Criteria for Adverse Events [CTCAE] criteria) at Screening; donation of blood or plasma within 28 days prior to randomization; or symptomatic loss of blood or hemorrhage within 28 days prior to randomization. 5. Patients with severe hemoptysis within 28 days prior to randomization, defined as >100 mL, (approximately >7 tbsp blood) over any 24-hour period. 6. Patients with severe hepatic impairment, as evidenced by alanine aminotransferase (ALT) or aspartate aminotransferase (AST)>3×upper limit of normal (ULN) or total bilirubin >2×ULN, or clinical signs of cirrhosis or end-stage hepatic disease (eg, ascites, hepatic encephalopathy). 7. Patients who are pregnant or breastfeeding. 8. Patients with a mean QT interval corrected using Fridericia's formula (QTcF)>480 msec based on triplicate 12-lead ECGs at Screening. 9. Patients with an immunodeficiency or an immunocompromised condition and risk for an opportunistic pulmonary infection, including: known history of human immunodeficiency virus (HIV) infection plus either an active acquired immunodeficiency syndrome (AIDS)-defining illness in the past 12 months, or a known cluster of differentiation 4 (CD4) count <200/mm³ within the past 12 months; neutropenia at Screening (absolute neutrophil count <1,000 neutrophils/mm³); or initiation of immunosuppressive therapy within 8 weeks prior to randomization, including cancer chemotherapy, transplant rejection medication, or chronic use of systemic corticosteroids defined as ³20 mg/day of prednisone or systemic equivalent. 10. Patients with an anticipated start of new non-study antimycobacterial therapy to be administered at any time between Screening and Month 6 (Day 169±7 days). 11. Patients who have received any investigational medication during the 30 days or 5 half-lives, whichever is longer, prior to randomization. 12. Patients with any prior exposure to epetraborole. 13. Patients with any condition that, in the opinion of the Investigator, interferes with the ability to safely complete the study or adhere to study requirements, including the patient's inability or unwillingness to comply with all study assessments and visits.

Withdrawal from Study

Participation of a patient in this clinical study may be discontinued for any of the following reasons, including, but not limited to: —The patient withdraws consent or requests discontinuation from the study for any reason; —The patient is lost to follow-up; —The patient fails to comply with protocol requirements or study-related procedures; —The Investigator determines that it is in the best interest of the patient to withdraw from the study protocol, for reasons other than an AE (eg, an occurrence of any medical condition or circumstance that exposes the patient to substantial risk and/or does not allow the patient to adhere to the requirements of the Protocol); —The study is terminated or temporarily suspended by the Sponsor or a regulatory authority for any reason, including, but not limited to, study drug-related unexpected life-threatening serious AEs (SAEs) detected during safety monitoring.

### Premature Discontinuation of Study Drug

Premature discontinuation of study drug by the Investigator is an important discussion that should include the Medical Monitor, if feasible, before study drug is discontinued. Possible reasons for premature discontinuation of study drug due to safety reasons include, but are not limited to, the following: —Occurrence of an AE that, in the opinion of the Investigator, warrants the patient's permanent discontinuation from study drug administration; —Hy's law criteria are met, defined by at least 3-fold elevations of ALT or AST>ULN, elevation of serum total bilirubin to >2×ULN without elevated serum alkaline phosphatase, and no other disease or condition can be found to explain the liver test abnormalities; —Known pregnancy or breastfeeding during the study drug administration period; —Decline in postbaseline renal function with an estimated CrCl less than or equal to 50 mL/min; —Persistence of positive sputum cultures beyond Month 6 of therapy; —Screening sputum sample cultures finalized as no growth (culture-negative) for MAC after randomization. Note: Reporting of final respiratory culture and identification results may occur up to 8 weeks after specimen receipt at the central laboratory. Unless the patient withdraws consent, patients who prematurely discontinue study drug will continue in the study for safety follow-up (ie, will not be withdrawn from the entire study) and will be assessed at the EOT and LFU Visits.

### Unit Dosage Forms & Route of Administration

The following unit dosage forms and routes of administration were utilized: Epetraborole oral tablets: 250 mg; Epetraborole dosage: 500 mg (two 250 mg oral tablets) once daily (QD); Placebo oral tablets: Matched to epetraborole tablets; Placebo regimen: Matched to epetraborole dosage (2 oral tablets QD).

Eligible patients will be randomized to 1 of the following treatment groups: a) Epetraborole oral tablets 500 mg (two 250 mg oral tablets) QD+OBR, or b) Placebo oral tablets QD matched to epetraborole dosage+OBR. In this study, OBR at the time of randomization must consist of at least 2 oral antimycobacterial drugs administered per current ATS/ERS/ESCMID/IDSA treatment guidelines.

A total of approximately 314 patients are enrolled in this Phase 2/3 study: The Phase 2 part of the study can consist of approximately 80 randomized patients (40 patients in the epetraborole+OBR group and 40 patients in the placebo+OBR group). The Phase 3 part of the study can consist of approximately 234 randomized patients (156 patients in the epetraborole+OBR group and 78 patients in the placebo+OBR group). The number of patients in the Phase 3 part of the study may be adjusted based on the results of Phase 2 analyses.

### Labeling, Packaging, Storage, and Handling

Epetraborole is provided for oral administration as a white to off-white modified-oval, film-coated, IR tablet containing 250 mg epetraborole as free base (288.5 mg as hydrochloride salt). Color-matched placebo tablets can also be provided.

### Study Drug Administration

Active epetraborole and matching placebo oral tablets can be administered at a dose of 500 mg QD. Study drug tablets can be administered in a fasting state (no food for at least 1 hour before and at least 2 hours after study drug administration). If GI upset occurs with study drug on an empty stomach, study drug administration may be taken with a small snack or administrated at bedtime to improve tolerability.

The first dose of study drug can be administered following randomization on Day 1 in the presence of the Investigator or designee. Following the first dose of study drug, patients will self-administer study drug orally QD.

### Interactive Responsive Technology and Allocation of Study Drug

An IRT system will be used for study drug management tasks. This may include randomization, study drug supply management, inventory management and supply ordering, study drug expiration tracking, and emergency unblinding.

Once informed consent has been obtained, patients will be assigned a unique 9-digit patient identifier at Screening.

The first approximately 80 patients of the study will be randomized for Phase 2 in a 1:1 ratio (40 patients receiving active epetraborole tablets and 40 patients receiving matching placebo tablets) using an IRT system. For the Phase 3 part of the study, approximately 234 patients are planned to be randomized in a 2:1 ratio (156 patients receiving active epetraborole tablets and 78 patients receiving matching placebo tablets) using an IRT system.

### Timing of Study Drug Dose Administration

The first dose of study drug can be administered as soon as possible following randomization on Day 1, in the presence of the Investigator or designee. Following the first dose of study drug, adjustments in the time of daily study drug administration are allowed to align with patient-specific medication dosing schedules in the management of treatment-refractory MAC lung disease.

Study Drug Dosing in Patients with Normal Renal Function or Mild Renal Impairment

Patients with CrCl≤50 mL/min are excluded from the study. Upon enrollment into the study, patients will undergo regular safety assessments, including routine monitoring of renal function (eg, serum creatinine) during the study.

Renal function, as estimated by CrCl, will be assessed using the Cockcroft-Gault formula and serum creatinine levels obtained at the central laboratory prior to Day 1 and as needed throughout the study per routine standard of care; in addition, CrCl will be programmatically determined from serum creatinine levels measured by the central laboratory.

$$\\ Cockcroft - Gault formula : Estimated CrCl ( mL / min ) =$$

$$\\ ( 140 - Age [ years ] ) \times Actual Body Weight [ kg ] \times [ 0.85 if Female ] ) /$$

$$\\ ( 72 \times Serum Creatinine [ mg / dL ] )$$

Estimated CrCl can be calculated every time a local laboratory assessment of serum creatinine is performed. Actual weight in kilograms (not ideal weight) is required for the CrCl calculation. If available, the weight obtained on the day of the serum creatinine measurement can be used for calculating CrCl; however, the baseline weight or most recent weight may be used throughout the study for estimated CrCl calculations, if repeated weights cannot be obtained.

After Day 1, if the estimated CrCl decreases to 50 mL/min during the Treatment Period, serial serum creatinine levels and CrCl estimations should be performed to determine whether study drug should be suspended or prematurely discontinued. The Investigator may choose to continue to monitor the patient and reassess the need for premature discontinuation of study drug based on subsequent CrCl assessments. If repeated CrCl estimations are ≤50 mL/min, study drug can be discontinued.

Study Drug Dosing in Patients with Anemia

Patients with baseline hemoglobin <10.0 g/dL or <6.2 mmol/L (Grade 2 anemia or worse, based on CTCAE criteria) can be excluded from the study. Decreases in RBC parameters (including hemoglobin, RBCs, and reticulocyte counts) have been reported in subjects receiving IV or oral epetraborole. In previous Phase 1 and Phase 2 studies of IV or oral epetraborole, reticulocyte decreases were observed within the first several days of dosing, followed by a decrease in hemoglobin. These hematological abnormalities appear to be transient, occur gradually, and when followed over time, return to baseline values after discontinuation of epetraborole.

Anemia is predefined as an Adverse Event of Special Interest. If anemia occurs during the study, hematology values should be rechecked at the local laboratory to verify the result, and perform a workup to determine the cause of anemia according to his/her best clinical judgment (including, but not limited to, iron deficiency [eg, check a serum iron panel], GI bleeding, menstruation, concomitant medications [eg, non-steroidal anti-inflammatory drugs, rifamycins, oxazolidinones, clofazimine], vitamin deficiencies [eg, vitamin B₁₂, folate], and hemolysis [eg, assess haptoglobin, blood smear]). As epetraborole has the potential to affect only erythropoiesis, and not the production of other blood cell types (eg, lymphocytes, basophils, eosinophils, neutrophils, monocytes, or platelets), anemia with concomitant decreases in white blood cells or platelets should prompt investigation of other potential causes.

If Grade 3 anemia or worse (hemoglobin <8.0 g/dL or <4.9 mmol/L) is identified at any time after initiation of blinded study drug treatment, the Investigator should recheck hematology values at the local laboratory to verify the result and perform a workup to determine the cause of the anemia as described above. If postbaseline Grade 3 anemia or worse is considered by the Investigator to be related to blinded study drug, administration of study drug should be temporarily discontinued. Interruption should not exceed more than 6 weeks. In such cases, hemoglobin levels should be monitored approximately every 1 to 2 weeks to ensure improvement by at least 1 grade.

9, All clinically significant abnormal hematology values should be followed until repeat tests return to normal, stabilize, or are no longer clinically significant. After improvement or resolution of anemia occurs, the case should be discussed with the Medical Monitor prior to restarting study drug. Case discussion with the Medical Monitor should occur prior to any study drug interruption or premature discontinuation.

### Dosing Interruptions, Incomplete Doses, and Missed Doses

Instances of noncompliance with the pre-specified dosage regimen (oral study drug dosed QD) will be documented as protocol deviations, including missed doses, or doses administered out of window.

If dosing is interrupted or incomplete, no adjustment to the dosing schedule is required.

If dosing is missed for any reason, the next planned dose should be administered as quickly as possible at the time of discovery, and the remainder of the doses should be administered at the pre-planned daily intervals starting with the following day (ie, the previous dosage schedule should remain unchanged).

Investigators are encouraged to discuss continued study drug administration options after interrupted dosing, incomplete, or missed doses with the Medical Monitor on a case-by-case basis.

### Prior and Concomitant Medications and Prohibited Treatment

### Prior Therapy

For purposes of evaluating inclusion criteria, all prior treatment includes all treatment (including over-the-counter treatments such as herbal supplements, vitamins, diet aids, and hormone supplements) received within 6 weeks prior to the date and time of first dose administration of study drug. Patient record of prior treatment can be documented in the appropriate eCRF.

### Concomitant Therapy

Concomitant medications or treatments administered between the dates and times of the first dose of study drug and the last study visit, inclusive, are to be listed in the appropriate eCRF. Concomitant administration of new, additional non-study-specific, antimycobacterial therapy that is potentially effective against MAC for reasons other than microbiological failure of study drug (in treating the index MAC lung infection) is not allowed; however, changes in the dosages of existing antimycobacterial agents are allowed, as needed. Any questions regarding the use of concomitant non-study-specific systemic antibiotic therapy can be directed to the Medical Monitor prior to administration.

### Prohibited Therapy

The following medications and treatments are prohibited: —Any anticipated or planned lung surgery for MAC lung disease; —Immunosuppressive therapy use within 8 weeks prior to randomization and throughout the treatment period, including cancer chemotherapy, transplant rejection medication, or chronic use of systemic corticosteroids defined as greater than or equial to 20 mg/day of prednisone or systemic equivalent; —New, additional non-study-specific, antimycobacterial therapy that is potentially effective against MAC for reasons other than microbiological failure of study drug (in treating the index MAC lung infection) at any time between Screening and Month 6 (Day 169±7 days; blinded study drug will be discontinued in patients who remain MAC culture-positive despite 6 months of therapy with study drug [based on Month 4 sputum culture results]), Note: Any changes in the dosages of existing antimycobacterial agents are allowed, as needed; —Any investigational medication during the 30 days or 5 half-lives, whichever is longer, prior to randomization and throughout the treatment period.

A patient who receives prohibited therapy can be excluded from the Per-Protocol Population. Epetraborole should be used with caution in patients with pre-existing anemia, those receiving concomitant drugs that cause anemia, or those with a comorbid condition that is not related to the condition under study and may be significantly compromised by a potential decrease in hemoglobin. Any questions regarding the use of concomitant non-study-specific therapy should be directed to the Medical Monitor prior to administration.

### Permitted Concomitant Therapy

With the exception of prohibited concomitant medications recited herein, other concomitant medications are permitted.

### Guidance for Investigators: ALIS (Amikacin Liposome Inhalation Suspension) Use Before and During the Study

For ALIS use at baseline, the following should be considered: —If ALIS has not been administered prior to study enrollment: As ALIS is recommended for use in treatment-refractory MAC lung disease according to the current treatment guidelines, the Investigator and potential patient must consider its use instead of study enrollment. If the decision is made to enroll the patient in this study without prior administration of ALIS therapy, the rationale for not administering ALIS must be clearly stated in the source documentation (eg, patient decision, Investigator decision, contraindication to use, not available, high apriori likelihood of an ALIS-associated AE). In an exemplary embodiment, neither ALIS nor any other new MAC-specific therapy, other than blinded study drug, can be started during the treatment phase of the study; —If ALIS was administered prior to study consideration and will be continued during the study: The patient may remain eligible for the study provided the microbiological criteria are met; —If ALIS was administered prior to study consideration and subsequently discontinued for any reason: The patient may remain eligible for the study provided the microbiological criteria are met and ALIS was discontinued at least 30 days prior to randomization.

### Safety and Efficacy Assessments

### Tests and Examinations Physical

### Examinations

A physical examination of the head (external), eyes, ears, nose and throat, lungs, cardiovascular system, abdomen, musculoskeletal system, skin, lymph nodes, central nervous system, and, as appropriate, other body systems can be performed at Screening and at every scheduled study visit. Physical examinations can be performed before the administration of study drug at the treatment visits. Clinically significant findings can be recorded in the eCRF as either a Medical History or as an AE.

### Vital Signs, Height, Weight, and BMI

Vital signs can be measured at Screening and at scheduled study visits. Vital signs can include temperature, heart rate, and blood pressure. Wherever possible, vital signs can be obtained after at least 5 minutes resting in a supine or sitting position. All findings must be recorded in the eCRF. Height (at Screening only; without shoes), weight, and body mass index (BMI) will also be measured and recorded at relevant time points.

### 12-Lead ECGs

Twelve-lead ECGs can be conducted at relevant time points. At the Screening Visit, triplicate ECGs can be obtained within an approximate 15-minute period, separated by approximately 1 minute. On Day 1, a standard single 12-lead safety ECG can be collected between the first dose of study drug and the blood PK sample (2 to 3 hours after dose administration). At other scheduled visits, a standard single 12-lead safety ECG can be collected without regard to study drug timing. Whenever possible, ECGs can be obtained after at least 5 minutes resting in a supine or semi-recumbent position. Findings can be recorded in the eCRF.

### Radiographic Assessments (Chest CTs)

Chest CT scans can be evaluated to assess eligibility and pulmonary radiographic response during the study. Study chest CTs, including Screening chest CT scans, can consist of contiguous sections through the lungs, each section of less than or equal to 3 mm thickness per local standard of care. Low-dose CT scans are acceptable. Digital images of chest CTs can be evaluated and interpreted at a central site by an independent blinded radiologist.

Radiographic response can be evaluated based on severity, extent of pulmonary disease, and overall improvement between baseline (Screening CT scan) and Month 6 and between baseline and EOT. Note: The interpretation of Screening chest CTs (to assess for abnormalities consistent with MAC lung disease) can be performed locally by the site (eg, Investigator or local radiologist). The exploratory endpoint of radiographic response at Month 6 and EOT based on overall change from baseline will be performed by an independent blinded radiologist.

Pre-Study chest CTs (performed per standard of care) may be used to determine eligibility if taken within the 8-week Screening Period and serve as the Screening CT. If the most recent Pre-Study chest CT was taken more than 8 weeks prior to randomization, a chest CT can be obtained within the Screening Period.

A minimum of 8 weeks between 2 chest CT scans is recommended. For example, if EOT occurs before Month 6, the EOT chest CT can be scheduled to occur at least 8 weeks after the Screening chest CT. If the last dose of study drug occurs within 8 weeks after the Month 6 visit (during which a chest CT is performed), the EOT chest CT can be scheduled to occur at least 8 weeks after the Month 6 chest CT.

### Clinical Laboratory Evaluations

Clinical laboratory evaluations can be performed at the relevant time points. Hematology and chemistry be performed for patients at the initial Screening Visit (ie, Days −56 to −15) to confirm eligibility. The only clinical laboratory requirement at the second Screening Visit (Days −14 to −7) is for patients with an initial Screening hemoglobin of <LLN to 10.0 g/dL or <LLN to 6.2 mmol/L (ie, Grade 1 anemia or worse, based on Common Terminology Criteria for Adverse Events) for confirmation of eligibility.

All clinical laboratory samples collected for eligibility and safety evaluations can be analyzed by a certified central clinical laboratory. Clinical laboratory evaluations required to confirm eligibility (ie, inclusion/exclusion criteria) include: —Serum creatinine (for calculation of CrCl using the Cockcroft-Gault formula); —Hemoglobin; —ALT; —AST; —Total bilirubin; —Absolute neutrophil count; —CD4 count (if HIV-positive and unknown CD4 count within the past 12 months); and —Serum pregnancy test (only if FOCP).

Day 1 clinical safety laboratory evaluations can be collected prior to the first dose of study drug on Day 1. Following study drug dosing, the Investigator will review laboratory values for those outside of normal range and can conduct clinically appropriate follow-up procedures. Clinical significance of the values outside of normal ranges can be assessed by the Investigator.

### Alcohol DeHydrogenase (ADH) Genotyping

ADH genotyping can occur on Day 1. ADH genetic variants can be tested to determine effects on PK analysis results; the results are not needed prior to dosing and do not impact study eligibility.

### Sputum Collection and Microbiological Assessments

Appropriate respiratory specimens can be collected during the Screening Period and at designated time points; 2 to 3 sputum samples can be collected at each time point and may be collected on the same day or separate consecutive days. Induced or expectorated sputum samples are required at relevant time points, except the Pre-Study respiratory specimen may be either an induced or expectorated sputum sample or a deep bronchial specimen.

Sputum samples collected during the Screening Period and at designated time points during the study can be sent to the central microbiological laboratory for mycobacterial culture, identification, quantification, susceptibility testing, and molecular testing. Sputum specimens may be obtained by the patient at home after site personnel training on the proper sputum collection and storage techniques. Induced sputum is the preferred method, whenever possible and safe. Sputum specimens can be refrigerated (not frozen) without fixatives or preservatives until shipped to the central microbiological laboratory. If a patient is unable to produce a postbaseline sputum specimen despite reasonable efforts (eg, due to a non-productive cough or resolution of cough), the reason the culture was not performed can be recorded.

The 8-week Screening Period allows for culture and identification of MAC isolates. Susceptibility and molecular testing results are not required prior to randomization. All isolates can be identified to the species level; however, only identification to MAC level rather than species level is required for randomization. The time from receipt of sputum at the central microbiological laboratory to a MAC-positive culture averages 3 weeks but may take up to 6 weeks. In addition, identification of MAC isolates to the species level using the line probe assay (LPA), averages 1 to 3 days. Isolates that cannot be identified or speciated using LPA can be identified and speciated via sequencing of rpoB gene or 16S rRNA, which takes up to an additional 7 days.

Microbiological evaluation of sputum samples can include improvement in quantitative colony counts. Quantification of bacteria via colony counts on solid agar can be performed and assigned a categorical score similar to that reported by Griffith, et al. Semiquantitative culture analysis during therapy for Mycobacterium avium complex lung disease. Am J Respir Crit Care Med. 2015; 192(6):754-760, where colony counts were predictive of symptomatic and radiographic improvement. An improvement in quantitative

TABLE 1
Categorical Scoring for Quantitative Culture Counts

| Colonies (CFU/mL) | Categorical Culture Score [a] |
|---|---|
| No growth on agar or broth | 0 |
| Growth in broth only | 1 |
| 1-49 | 2 |
| 50-99 | 3 |
| 100-199 | 4 |
| 200-299 | 5 |
| ≥300 | 6 |

Abbreviations: CFU = colony-forming units.
[a] Categorical scores based on those reported in Griffith 2015.

culture counts will be defined as a reduction in greater than or equal to 1 categorical score from the previous month's categorical culture score.

The central microbiological laboratory follows standardized methods and procedures for MAC culture, identification, and susceptibility testing (following Clinical & Laboratory Standards Institute (CLSI) guidelines). All respiratory culture specimens can be cultured in appropriate liquid and solid media. If the results are negative on solid media, the liquid media can be held for 6 weeks before reporting the culture as negative.

Standard antibiotic susceptibility testing can be performed using the most recent CLSI guidelines at the central microbiological laboratory. Epetraborole MIC values are not reported to local sites during the study. Decreases in susceptibility of postbaseline isolates to epetraborole can be determined based on an increase in MIC values of ³4-fold above those obtained at baseline. Postbaseline isolates with decreased susceptibility and the corresponding baseline isolate can be retested twice to confirm MIC values. If the result is confirmed, molecular testing can be performed to determine relatedness of postbaseline to baseline isolate.

Molecular testing, including whole genome sequencing, can be performed to determine genetic relatedness of select baseline/postbaseline isolates. Analyses include, but are not limited to, comparison of patients' postbaseline MAC isolates to the baseline MAC isolate to determine if isolates are clonal and suggestive of persistent infection, relapse, or reinfection with an isolate of a different genotype. In this study, as per the NTM Network European Trials Group consensus statement on NTM treatment outcome definitions, “reinfection” is defined as a new pulmonary MAC infection caused by strain(s) different from the baseline MAC strain as determined by molecular typing, and “relapse” is defined as pulmonary MAC infection caused by the same baseline MAC strain as determined by molecular typing. Whole genome sequencing may also be utilized to evaluate the presence of genetic markers of antibacterial resistance.

### Plasma PK Sampling and Assessments

Blood samples for PK analyses can be collected from all patients in a blinded manner. Blood samples can be collected after administration of study drug in a fasted state.

TABLE 2

| Sample | Sampling Time |
|---|---|
| Sample 1 | 2-3 hours after the first dose of study drug on Day 1, |
| | in a fasting state |
| Sample 2 | Within 2 hours before the dose planned on the day of |
| | the Day 29 visit (that day's dose will be held until |
| | this pharmacokinetic sample is collected) |
| Sample 3 | 2-3 hours after the dose administered on the day of |
| | the Day 29 visit, in a fasting state |

These samples can be used to estimate epetraborole PK parameters, including AUC, Cₘₐₓ, time to reach Cₘₐₓ (Tₘₐₓ), total body clearance from plasma (CL), t_{1/2}, minimum plasma drug concentration (Cₘᵢₙ), and apparent volume of distribution at steady state (Vₛₛ). Plasma PK samples obtained from the epetraborole group can be analyzed using a validated assay by a central bioanalytical laboratory. Samples from placebo-treated patients may not be analyzed.

### Schedule of Assessments: Screening Through Month 6 (Phases 2 and 3)

| | | | | Treatment Period | | | | | | | | |
|---|---|---|---|---|---|---|---|---|---|---|---|---|
| | | | | Month | | | | | | | | |
| | | | | Random- | | | | 3 + | | | | 6 + |
| | | Screening | | ization | 1 | 2 | 3 | 1 w | 4 | 5 | 6 | 1 w |
| | | Study Day/Visit Window^{a} | | | | | | | | | | |
| | | D −56 | D −14 | | D 29 ± | D 57 ± | D 85 ± | | D 113 ± | D 141 ± | D 169 ± | |
| | | to −15 | to −7 | D 1 | 7 d | 7 d | 7 d | D 92 | 7 d | 7 d | 7 d | D 176 |
| | Informed consent^{b} | X | | | | | | | | | | |
| | Inclusion/exclusion criteria | X^{c} | | | | | | | | | | |
| | Demographic information | X | | | | | | | | | | |
| | Medical/surgical history | X | | X | | | | | | | | |
| Tests/Exams | Physical examination^{d} | X | | X | X | X | X | | X | X | X | |
| | Vital signs, weight^{e} | X | | X | X | X | X | | X | X | X | |
| | Height, BMI^{e} | X | | | | | | | | | | |
| | 12-lead ECG^{f} | X | | X | | X | | | X | | X | |
| | Chest CT^{g} | X | | | | | | | | | X | |
| Laboratory | Chemistry/Hematology^{h} | X^{c} | Xⁱ | X^{j} | X | X | X | | X | X | X | |
| | Urinalysis^{h} | X | | X^{j} | | X | | | X | | X | |
| | Serum pregnancy test | X | | X^{j} | | | | | | | | |
| | (FOCP only)^{h} | | | | | | | | | | | |
| | CrCl calculation^{k} | X | | | | | | | | | | |
| | ADH genotyping^{l} | | | X | | | | | | | | |
| | Sputum collection ^{m} | Xⁿ | | | X | X | X | | X | X | X | |
| | Plasma PK sampling^{o} | | | | | | | | | | | |
| | Randomization | | | X | | | | | | | | |
| | Study drug administration | | | Daily^{p} | | | | | | | | |
| | Prior/concomitant | X | X | X | X | X | X | | X | X | X | |
| | medications | | | | | | | | | | | |
| | Adverse events^{q} | X | X | X | X | X | X | | X | X | X | |

Abbreviations: ADH = alcohol dehydrogenase; AE = adverse event; BMI = body mass index; CrCl = creatinine clearance; CT = computed tomography; D = Day; ECG = electrocardiogram; EOT = End of Therapy; ET = Early Termination; FOCP = female patient of childbearing potential; MAC = Mycobacterium avium complex; NTM = Nontuberculous mycobacteria; PK = pharmacokinetic(s); QD = once daily; SAE = serious adverse event; TEAE = treatment-emergent adverse event; w = week.
NOTE:
See Table for Months 7-16, EOT/ET, and LFU assessments.
^{a}Study days are anchored to Day 1.
^{b}Signed informed consent: Must obtain before any study-related procedures are performed.
^{c}Laboratory tests required for eligibility are listed herein.
^{d}Physical Exam: perform before the administration of study drug at the treatment visits. Vital Signs, Height (without shoes), Weight, BMI: described herein, when possible, obtain vitals after at least 5 minutes resting in a supine or sitting position.
^{e}ECGs: Listed herein, including timing of Screening triplicate ECGs and single ECGs thereafter. Whenever possible, collect ECGs after at least 5 minutes resting in a supine or semi-recumbent position.
^{f}Chest CTs: Details provided herein, including timing of Pre-Study chest CTs (for eligibility).
^{g}Laboratory samples (central laboratory): Details provided herein.
^{h}The only clinical laboratory requirement at the second Screening Visit is for patients with an initial Screening hemoglobin of <LLN to 10.0 g/dL or <LLN to 6.2 mmol/L (ie, Grade 1 anemia or worse, based on CTCAE) for confirmation of eligibility.
ⁱLaboratory samples: On Day 1, collect prior to first dose of study drug.
^{j}CrCl Calculation: Use the Cockcroft-Gault formula and serum creatinine levels obtained at the central laboratory prior to Day 1 and as needed throughout the study per routine standard of care. Calculate estimated CrCl every time a local laboratory assessment of serum creatinine is performed.
^{k}ADH genotyping: Genetic variants will be tested to determine effects on PK analysis results; the results are not needed prior to dosing and do not impact study eligibility.
^{l}Sputum Samples (central microbiological laboratory): Details provided herein; collect 2 to 3 sputum samples at each time point; may be collected on the same day or separate consecutive days.
^{m} Screening Sputum Samples (central microbiological laboratory): Details provided herein; in addition to the Pre-Study MAC-positive respiratory specimen, 1 Screening MAC-positive expectorated or induced sputum (not deep bronchial) sample must be collected within the 8-week Screening Period.
ⁿPlasma PK Sampling (central bioanalytical laboratory): Collect PK blood samples from all patients in a blinded and fasting manner.
^{o}Study Drug Administration: Tablets can be administered QD in a fasting state (no food for at least 1 hour before and at least 2 hours after study drug administration; with the exception of PK sample collection as shown in Table 2; patients may eat after PK sample collection). The first dose of study drug can be administered in the presence of the Investigator or designee following randomization.
^{p}AE Collection: Details provided herein on collection, monitoring, and reporting of TEAEs, including SAEs.

### Schedule of Assessments: Months 7-16, EOT, and LFU (Phases 2 and 3)

| | | Treatment Period^{a} | | | | | | | | |
|---|---|---|---|---|---|---|---|---|---|---|
| | | Month | | | | | | | | |
| | | 7, 8^{b} | 9 | 10, 11^{b} | 12 | 13, 14^{b} | 15 | 16 | | |
| | | Study Day/Visit Window^{c} | | | | | | | | |
| | | | | D 281, | | D 365, | | | EOT | |
| | | D 197, | D 253 ± | 309 ± | D 337 ± | 393 ± | D 421 ± | D 449 ± | Visit/ | LFU |
| | | 225 ± 7 d | 7 d | 7 d | 7 d | 7 d | 7 d | 7 d | ET^{d} | Visit^{e} |
| Tests/Exams | Physical examination^{f} | | X | | X | | X | | X | X |
| | Vital signs^{g} | X | X | X | X | X | X | X | X | X |
| | Weight | | X | | X | | X | | X | X |
| | 12-lead ECG^{h} | | | | | | | | X | |
| | Chest CTⁱ | | | | | | | | X | |
| Laboratory | Chemistry/Hematology^{j} | X | X | X | X | X | X | X | X | X |
| | Urinalysis^{j} | | X | | X | | X | | X | X |
| | Serum pregnancy test (FOCP | | | | | | | | X | |
| | only)^{j} | | | | | | | | | |
| | Sputum collection^{k} | | X | | X | | X | | X | X |
| | Study drug administration | Daily^{a, l} | | | | | | | | |
| | Prior/concomitant medications | X | X | X | X | X | X | X | X | X |
| | Adverse events^{m} | X | X | X | X | X | X | X | X | X |

Abbreviations: AE = adverse event; CT = computed tomography; D = Day; ECG = electrocardiogram; EOT = End of Therapy; ET = Early Termination; FOCP = female patient of childbearing potential; HHC = home health care; LFU = Late Follow-up; MAC = Mycobacterium avium complex; NTM = Nontuberculous mycobacteria; QD = once daily; SAE = serious adverse event; TEAE = treatment-emergent adverse event.
^{a}Each patient's duration of treatment will vary depending on the timing of their MAC culture conversion. A patient may not qualify for visits beyond Month 6 if they did not have 3 consecutive negative culture results by Month 6.
^{b}Visit may be conducted via HHC, if available.
^{c}Study days are anchored to Day 1.
^{d}EOT Visit: Perform within 7 calendar days after the last dose of study drug. The EOT assessments may be performed during a regularly scheduled clinic visit if within the EOT window. Patients with clinically significant abnormal laboratory values at EOT that are deemed possibly or probably related to study drug will have repeated laboratory assessments until values return to normal or pre-study baseline values. For patients who are withdrawn from the study prior to completion, all the EOT Visit procedures will be performed at an Early Termination Visit.
^{e}LFU Visit: Perform 3 months (84 days ± 14 days) after the last dose of study drug.
^{f}Physical Exam: Details provided herein; perform before the administration of study drug at the treatment visits.
^{g}Vital Signs: Details provided herein; when possible, obtain vitals after at least 5 minutes resting in a supine or sitting position.
^{h}ECGs: Details provided herein; when possible, collect ECGs after at least 5 minutes resting in a supine or semi-recumbent position.
ⁱChest CTs: Details provided herein; for details, including timing of EOT CTs.
^{j}Laboratory samples (central laboratory): Details provided herein and list of analytes.
^{k}Sputum Samples (central microbiological laboratory): Details provided herein; collect 2 to 3 sputum samples at each time point; may be collected on the same day or separate consecutive days.
^{l}Study Drug Administration: Details provided herein; tablets to be administered QD in a fasting state (no food for at least 1 hour before and at least 2 hours after study drug administration). The first dose of study drug will be administered in the presence of the Investigator or designee following randomization.
^{m}AE Collection: Details provided herein on collection, monitoring, and reporting of TEAEs, including SAEs.

APPENDIX
Clinical Laboratory Analytes

| Hematology | Serum Chemistry | Urinalysis |
|---|---|---|
| Red blood cell (RBC) count | Liver Tests: | pH |
| Red cell distribution width | Albumin | Specific gravity |
| Erythropoietin levels | Alkaline phosphatase (ALP) | Bilirubin |
| Reticulocyte count (% and | Alanine transaminase (ALT) | Blood |
| absolute) | Aspartate transaminase (AST) | Glucose |
| White blood cell (WBC) | Bilirubin direct | Ketones |
| count and differential | Bilirubin, indirect | Leukocyte esterase |
| Hemoglobin (HGB) | Bilirubin, total | Microscopy |
| Hematocrit | Gamma-glutamyl | Nitrite |
| Platelet count | transferase (GGT) | Protein |
| Mean corpuscular volume | Amylase | Urobilinogen |
| Mean corpuscular HGB | Anion gap | Other |
| Mean corpuscular HGB | Bicarbonate | Serum β-human chorionic |
| concentration | Adjusted calcium | gonadotropin (FOCP only) |
| Mean platelet volume | Blood urea nitrogen (BUN) | Alcohol dehydrogenase |
| Iron Studies: | or urea | (ADH) genotyping |
| Iron | Calcium | CD4 (only if HIV-positive |
| Transferrin | Chloride | and unknown CD4 count |
| Transferrin saturation | Cholesterol | within the 12 months prior |
| Ferritin | Creatine kinase | to Screening [to determine |
| | Creatinine | eligibility]) |
| | Creatinine clearance | |
| | (calculated using | |
| | Cockcroft-Gault formula) | |
| | Folate | |
| | Globulin | |
| | Glucose | |
| | Lactate dehydrogenase | |
| | Phosphorus, inorganic | |
| | Potassium | |
| | Protein, total | |
| | Sodium | |
| | Uric Acid | |
| | Vitamin B12 | |

### Pairwise Combinations of Epetraborole

Epetraborole hydrochloride is a small polar molecule with a novel mechanism of action (MoA) that has broad-spectrum antibacterial activity including activity against mycobacteria. The current standard of care for Mycobacterium avium complex (MAC) pulmonary disease involves azithromycin (or clarithromycin), ethambutol and a rifamycin. The activity of epetraborole hydrochloride was assessed in pairwise combinations with clarithromycin, rifabutin and ethambutol against 7 nontuberculous mycobacteria including 5 slowly growing nontuberculous mycobacteria and 2 rapidly growing mycobacteria. Epetraborole hydrochloride activity was also assessed in the presence of amikacin and bedaquiline.

### Materials and Methods

### Chemicals

EBO (epetraborole hydrochloride) was obtained from AN2 Therapeutics Inc, bedaquiline was purchased from 1Click Chemistry (Kendall Park, NJ), clarithromycin was purchased from Carbosynth (San Diego, CA), amikacin, ethambutol and rifabutin were purchased from Sigma-Aldrich, (St. Louis, MO). Frozen stocks of the drugs were prepared at 5 or 2 mg/ml and frozen in aliquots at −20° C. On the day of the experiment the stocks were thawed and diluted to the appropriate concentration. Middlebrook 7H9 broth and agar, CA-MH agar and Middlebrook OADC were all purchased from Becton-Dickinson and Company (Sparks, MD).

### Strains

M. avium 2285R (Verma et al. Microbiol (2019) 10: 693) and M intracellulare DNA000111 were obtained from Diane Ordway (Colorado State University, Fort Collins, CO). M. chimaera 20-S-05 and the macrolide/bedaquiline-resistant M. intracellulare 20-S-13 isolate were obtained from Barbara Brown-Elliott (Mycobacteria/Nocardia Research Laboratory, University of Texas Health Science Center, Tyler Tx). The strains M abscessus ATCC 19977, M. avium ATCC 700898 and M. peregrinum ATCC 700686 were obtained from the American Type Culture Collection (ATCC, Manassas, VA). Stocks of isolates were prepared and frozen at −80° C. Fresh cultures of each isolate (1-2 weeks) were grown on either 7H10 agar plus 5% OADC or CAMH agar plus 5% OADC for use in each experiment.

### Antibacterial Synergy Testing

The checkerboard methodology was used to test for synergy and antagonism using Middlebrook 7H9+5% OADC (Dubos et al. Am Rev Tuberc (1947) 56:334-45) as the microbiology growth media. The first antibiotic of the combination, EBO, was serially diluted along the x-axis, whereas the second antibiotic was diluted along the y-axis. Synergistic or antagonistic activity was determined using the sum of the fractional inhibitory concentration (ΣFIC) index. The FTC index is calculated as the sum of FTC A+FTC B, where FTC A is the MIC of drug A in the combination of drugs A and B divided by the MIC of drug A alone, plus the MIC of drug B in the combination of drugs A and B divided by the MIC of drug B alone. A combination of drugs is considered synergistic when the FIC is ≤0.5, additive when the FTC is >0.5 to 1, indifferent when the FIC is >1 to 2, and antagonistic when the FTC is >2 (EUCAST. Clin. Microbiol. Infect. (2000) 6: 503-508).

TABLE
Summary Activity of EBO Drug Combinations

| | | | | | | M. | M. |
|---|---|---|---|---|---|---|---|
| In | M. avium | | M. | M. | | abscessus | peregrinum |
| Combination | ATCC | M. avium | chimaera | intracellulare | M. intracellulare | ATCC | ATCC |
| with EBO | 700898 | 2285R | 20-S-05 | 20-S-13 | DNA000111 | 19977 | 700686 |
| CLR | I | A | I | NE | I | I | I |
| RFB | I | I | I | I | A | I | I |
| EMB | S | A | A | I | S | I | I |
| AMK | I | I | I | I | I | I | A |
| BDQ | A | I | I | NE | I | I | I |

AMK = Amikacin, BDQ = Bedaquiline, CLR = Clarithromycin, EBO = epetraborole hydrochloride, EMB = Ethambutol, RFB = Rifabutin, I = Indifferent, A = Additive, S = Synergistic. NE = No endpoint due to MIC of second drug being out of range, CLR MIC >128 mg/L, BDQ >0.5 mg/L.

### Results and Discussion

The in vitro activity of EBO was tested in the presence of key components of the standard of care drugs for the treatment of MAC pulmonary disease, clarithromycin, ethambutol, rifabutin as well as other known active NTM drugs, amikacin and bedaquiline. The activity of EBO was not antagonized by any of these drugs with any of the NTM strains we tested. In most cases, especially for the two rapidly growing NTM strains, M. abscessus ATCC 19977 and M. peregrinum ATCC 700686, EBO activity was indifferent to the addition of a second drug. The sole exception was ethambutol where synergy was observed with 2 strains and additivity with an additional 2 strains out of a total of 5 MAC strains tested. Interestingly, the clarithromycin resistant strain, M. intracellulare 20-S-13, was the only MAC strain that showed indifference between ethambutol and EBO and this strain had the highest ethambutol MIC with a value of 64 mg/L.

### CONCLUSIONS

No antagonisms were observed with any strain or EBO combination, and most interactions were largely indifferent especially for the two rapidly growing NTM strains, A abscessus ATCC 19977 and M. peregrinum ATCC 700686. The sole exception was ethambutol where synergy was observed with 2 strains and additivity with an additional 2 strains out of a total of 5 MAC strains tested. Interestingly, the MAC strain with the highest ethambutol MIC value of 64 mg/L was the only strain that showed indifference. Further testing would be required to determine how widespread EBO-Ethambutol synergy or additivity is for MAC.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof are encompassed by the disclosure unless otherwise indicated herein or otherwise clearly contradicted by context.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. A method of achieving a positive therapeutic response against a non-tuberculosis Mycobacteria (NTM) infection, comprising:
administering to a human in need thereof
epetraborole, or a hydrate, solvate, or pharmaceutically acceptable salt thereof, and
ethambutol or a hydrate, solvate, or pharmaceutically acceptable salt thereof, thereby achieving the positive therapeutic response.

2. The method of claim 1, wherein the epetraborole, or a pharmaceutically acceptable salt thereof, is administered in an oral dose of between about 250 mg and about 1000 mg.

3. The method of claim 1, wherein the epetraborole, or a pharmaceutically acceptable salt thereof, is administered in an oral dose of between about 450 mg and 550 mg.

4. The method of claim 1, wherein the epetraborole, or a pharmaceutically acceptable salt thereof, is administered in an oral dose of about 500 mg.

5. The method of claim 1, wherein epetraborole hydrochloride is administered to the human.

6. The method of claim 1, wherein the ethambutol or a pharmaceutically acceptable salt thereof, is administered in an oral dose of between about 10 mg/kg and about 30 mg/kg.

7.-10. (canceled)

11. The method of claim 1, wherein the administering continues until the positive therapeutic response is achieved.

12. The method of claim 1, wherein the administering continues after the positive therapeutic response is achieved.

13. The method of claim 1, wherein the administering is according to a dosage regimen comprising:
an oral dose of the epetraborole, or a pharmaceutically acceptable salt thereof, of between about 250 mg and about 1000 mg, administered daily or every other day, and
an oral dose of the ethambutol, or a pharmaceutically acceptable salt thereof, of between about 10 mg/kg and about 30 mg/kg, administered daily or three days a week.

14. The method of claim 1, wherein the administering is according to a dosage regimen comprising:
an oral dose of the epetraborole, or a pharmaceutically acceptable salt thereof, of between about 450 mg and about 550 mg, administered daily, and
an oral dose of the ethambutol or a pharmaceutically acceptable salt thereof, of between about 13 mg/kg and about 17 mg/kg, administered daily.

15. The method of claim 1, wherein the administering is according to a dosage regimen comprising:
an oral dose of the epetraborole or a pharmaceutically acceptable salt thereof, of between about 450 mg and about 550 mg, administered daily, and
an oral dose of the ethambutol, or a pharmaceutically acceptable salt thereof, of between about 23 mg/kg and about 27 mg/kg, administered three days a week.

16. The method of claim 13, wherein the dosage regimen lasts between about 27 and about 29 days.

17. The method of claim 16, wherein the dosage regimen is repeated until the positive therapeutic response is achieved.

18.-24. (canceled)

25. The method of claim 1, further comprising administering a rifamycin, or a hydrate, solvate, or pharmaceutically acceptable salt thereof, to the human.

26. The method of claim 25, wherein the rifamycin is rifampicin (rifampin), rifabutin, rifapentine, or rifaximin.

27.-32. (canceled)

33. The method of claim 1, further comprising administering azithromycin, or a hydrate, solvate, or pharmaceutically acceptable salt thereof.

34. (canceled)

35. (canceled)

36. The method of claim 1, further comprising administering clarithromycin or a hydrate, solvate, or pharmaceutically acceptable salt thereof.

37.-40. (canceled)

41. The method of claim 1, further comprising administering amikacin to the human.

42.-49. (canceled)

50. The method of claim 13, wherein the method further comprises:
prior to the first administration of the dosage regimen, assessing an infection marker of the human,
wherein the infection marker is quantitative colony count and/or chest CT scan, thereby obtaining a baseline assessment, and
after the first administration of the dosage regimen, assessing the infection marker of the human, thereby obtaining a response assessment,
wherein the infection marker selected for the response assessment is the same as selected for the baseline assessment,
comparing the baseline assessment and the response assessment, and
determining the positive therapeutic response is achieved when the response assessment is an improvement over the baseline assessment.

51. (canceled)

52. (canceled)

53. (canceled)

54. The method of claim 50, wherein the infection marker is expectorated sputum sample or induced sputum sample, and the response assessment is taken every between about 21 days and about 35 days after the first dose of each dosage regimen, for between about 84 days and about 166 days, and the positive therapeutic response is when the quantitative colony count in the response assessment is one or more categorical scores below the quantitative colony count in the baseline assessment.

55.-59. (canceled)

60. The method of claim 1, wherein the non-tuberculosis Mycobacteria is M. abscessus, M. avium complex (MAC), M. fortuitum complex, M. gordonae, M. kansasii, M. xenopi, M. arabiense, M. aromaticivorans, M. bacteremicum, M. barrassiae, M. bourgelatii, M. celeriflavum, M. chelonae, M. crocinum, M. franklinii, M. fukienense, M. hippocampi, M. insubricum, M. iranicum, M. litorale, M. llatzerense, M. monacense, M. pallens, M. rufum, M. rutilum, M. salmoniphilum, M. sediminis, M. setense, M. algericum, M. alsiense, M. arosiense, M. bouchedurhonense, M. engbaekii, M. europaeum, M. fragae, M. heraklionense, M. indicus pranii, M. koreense, M. kumamotonense, M. kyorinense, M. lepromatosis, M. liflandii, M. longobardum, M. mantenii, M. marseillense, M. minnesotense, M. noviomagense, M. paraffinicum, M. paragordonae, M. parakoreense, M. paraseoulense, M. paraterrae, M. riyadhense, M. senuense, M. seoulense, M. sherrisii, M. shigaense, M. shinjukuense, M. simulans, M. sinense, M. stomatepiae, M. timonense, M. vulneris, M. yongonense, M. mucogenicum, M. peregrinum, M. intracellulare, M. marseillaise, M. timonense, M. colombiense, or M. chimaera, or any combination thereof.

61. The method of claim 1, wherein the non-tuberculosis Mycobacteria is Mycobacterium avium complex.

62.-66. (canceled)

67. The method of claim 1, wherein the human has been administered at least one previous NTM treatment, which is not the epetraborole.

68. The method of claim 1, wherein the human has been administered at least two previous NTM treatments, neither of which is the epetraborole.
